(19) United States
(12) Patent Application Publication
(10) Pub. No.: US 2019/0111059 A1
(43) Pub. Date: Apr. 18, 2019

(54) COMPOSITIONS AND METHODS FOR TREATING AUTISM SPECTRUM DISORDER

(71) Applicant: Marinus Pharmaceuticals, Inc., Radnor, PA (US)

(72) Inventors: Richard Frye, Phoenix, AZ (US); John Slattery, Atlanta, GA (US)

(73) Assignee: Marinus Pharmaceuticals, Inc., Radnor, PA (US)

(21) Appl. No.: 16/163,021

(22) Filed: Oct. 17, 2018

Related U.S. Application Data
(60) Provisional application No. 62/573,493, filed on Oct. 17, 2017.

(51) Int. Cl. A61K 31/519; A61P 25/28; A61P 25/24; A61P 25/18; A61P 25/08; A61K 9/70

(52) U.S. Cl. CPC A61K 31/519; A61P 25/28; A61P 25/24; G01N 33/6854; A61P 25/08; A61K 9/7007; A61P 25/18

(57) ABSTRACT

A method of treating suffering from a CNS disorder, comprising administering a reduced folate, or a derivative, prodrug, active metabolite, stereoisomer, polymorph, analogue, or pharmaceutically acceptable salt thereof, to a human suffering from a neurobehavioral CNS disorder in which the patient exhibits a folate deficiency, wherein the amount of reduced folate is therapeutically effective to improve at least one core symptom of the CNS disorder, is disclosed.

### CROSS-REFERENCE TO RELATED APPLICATION

This application claims priority from U.S. Provisional Application No. 62/573,493, filed on Oct. 17, 2017, the disclosure of which is hereby incorporated by reference in its entirety for all purposes.

This invention was made with government support under NICHD ID: 1 R01 HD088528-01 awarded by the NICHD of the National Institutes of Health. The government has certain rights in the invention.

### BACKGROUND

Reduced levels of folates in central nervous system (“CNS”) are implicated in a number of neuro-psychiatric conditions and may be caused by a dysfunction in the folate receptor α (FRα) system, which is a high affinity capacity transporter with affinity for folic acid and methylfolate in the nanomolar range. The dysfunction is evidence by the presence of FRα autoantibodies (“FRAAs”).

Because levels of folates in CNS (e.g., in cerebrospinal fluid CSF)) do not necessarily correlate with levels of folate in serum, prior to the present invention, the only way to determine whether a subject has a low level of folates in CNS (e.g., in CSF) was to do a lumbar puncture to obtain a sample of CSF, which is a painful, invasive and expensive procedure, and measure a level of 5-methyltetrahydrofolate (“5-MTHF”) in the sample. A CSF 5-MTHF level of less than about 40 nmol/1 indicated that the subject has low level of folates in CNS.

Autism Spectrum Disorder (ASD) includes autistic disorder, Asperger's Syndrome, and pervasive developmental disorder—not otherwise specified (PDD-NOS) and is characterized by impairments in communication and social interaction, along with restrictive and repetitive behaviors. An estimated one out of 59 individuals in the U.S. is currently affected with an ASD.

Only two drugs have been approved by the United States Food and Drug Administration for the treatment of ASD. Both drugs are antipsychotic drugs indicated for associated, not core, ASD symptoms. However, these drugs can detrimentally affect lipid, cholesterol and glucose metabolism and can result in marked body weight gain; and can increase the risk of developing type 2 diabetes. Thus, well-tolerated medications that target pathophysiological processes and core symptoms associated with ASD are sorely needed. Methods for identifying these medications and using these medications to treat ASD are also needed. Some embodiments of the present invention are designed to meet these needs.

### SUMMARY OF THE INVENTION

The present invention provides means and methods for delivering folates into CNS, when the FRα system is impaired; and compositions, pharmaceutical compositions, methods for treating, methods for formulating, methods for producing, methods for manufacturing, treatment strategies, and pharmacokinetic strategies comprising and/or using reduced folate compounds (e.g., folinic acid, a folinic acid derivative, active metabolite, prodrug, stereoisomer, polymorph, analogue, pharmaceutically acceptable salts of all of the foregoing, etc.). The reduced folates are used for the treatment of at least one symptom (e.g., a core symptom) in a neurobehavioral CNS disorder (e.g., ASD). The present invention is further directed to methods and instruments for ascertaining degrees of verbal communication impairments.

In the present invention, the folates are delivered into CNS in the form of reduced folates (the opposite of oxidized folates found in food (e.g., folic acid)). Unlike oxidized folates, reduced folates do not require FRα for crossing the Blood Brain Barrier (BBB) and entry into CNS. Instead, reduced folates get transported to CNS (brain) by a reduced folate carrier (RFC), which is a low affinity, high capacity transporter that requires a substrate (a reduced folate) in the micro molar range. RFC facilitates the transfer of reduced folates into CNS even when FRα dysfunction is present.

Administration of reduced folates as described herein may correct the underlying deficiency of folates in CNS (e.g., CSF), which, in turn, may lead to an improvement in one or more core symptom(s) of ASD and other CNS disorders. The improved core symptoms may, e.g., be improved verbal communications, improved daily living skills, lessened irritability, lessened lethargy, lessened stereotyped behavior, lessened inappropriate speech, reduced hyperactivity, reduce internalization of problems, and improved socialization. The administration of a reduced folate (e.g., folinic acid) for 12 weeks in accordance with the present invention to a subject preferably results in a statistically significant improvement in a verbal communication score of the subject.

The present invention is considered to be useful in human patients of all ages. However, in certain embodiments, the invention is directed in part to the treatment of at least one core Autism Spectrum Disorder (ASD) symptom and other CNS disorders in human children that range in age from about 1 day to about 18 years old, from about 1 week to about 16 years old, from about 1 month to about 16 years old, from about 3 months to about 15 years old, or from about 3 years to about 14 years old.

In some embodiments, the invention allows for streamlining the treatment of the core symptom of ASD and other CNS disorders in a subject, because the invention avoids a need for determination of the CSF level of folates (e.g., MTHF) in the subject prior to the initiation of therapy with reduced folates and consequently avoids the discomfort and negative consequences of determining of CSF level of folates (e.g., MTHF) via, e.g., a lumbar puncture. Instead, the presence of the FRα antibodies in serum of the subject, independent of gene status, is used an indicator that administration of a high dose of a reduced folate (e.g., folinic acid) to the subject on a chronic basis may be warranted and/or may resolve or improve one or more core symptom(s) of ASD in the subject if such symptom(s) are present.

Thus, the present invention is directed in part to a method of treating suffering from a CNS disorder (e.g., a neurobehavioral, neurodevelopmental, or neurodegenerative disorder), including ASD, comprising administering a reduced folate, or a derivative, prodrug, active metabolite, stereoisomer, polymorph, analogue, or pharmaceutically acceptable salt thereof, to a human suffering from a neurobehavioral CNS disorder and having FRα antibodies in serum, wherein the amount of reduced folate is therapeutically effective to improve at least one core symptom of the CNS disorder. In certain preferred embodiments, the human has not been subjected to a lumbar puncture prior to said administration, did not receive a folic acid antagonist (e.g., methotrexate) and/or 5-fluorouracil prior to administration of the reduced folate (e.g., within 72 hours, 48 hours or 24 hours of administration of the reduced folate), does not have an anemia (e.g., megaloblastic or pernicious anemia), does not have a chronic fatigue syndrome, does not have cancer (e.g., colorectal cancer), is not pregnant, and is not malnourished.

The present invention is further directed in part to a method of treating suffering from a CNS disorder (e.g., a neurobehavioral, neurodevelopmental, or neurodegenerative disorder), including ASD, comprising administering a reduced folate, or a derivative, prodrug, active metabolite, stereoisomer, polymorph, analogue, or pharmaceutically acceptable salt thereof, to a human suffering from a neurobehavioral CNS disorder and having FRα antibodies in serum, wherein the amount of reduced folate is therapeutically effective to improve at least one core symptom of the CNS disorder, and the human has normal serum levels of folate (i.e., a serum level of folate of above 5 ng/ml and RBC folate level of above about 200 ng/ml (e.g., from about 225 ng/ml to about 600 ng/ml)).

The invention is further directed to a method of treating suffering from a neurobehavioral CNS disorder, including ASD, comprising administering a reduced folate, or a derivative, prodrug, active metabolite, stereoisomer, polymorph, analogue, or pharmaceutically acceptable salt thereof, to a human suffering from a neurobehavioral CNS disorder in which the patient exhibits a folate deficiency, wherein the amount of reduced folate is therapeutically effective to improve at least one core symptom of the neurobehavioral CNS disorder.

The present invention is also directed in part to a method of treating suffering from a CNS disorder (e.g., a neurobehavioral, neurodevelopmental, or neurodegenerative disorder), including ASD, comprising administering a reduced folate, or a derivative, prodrug, active metabolite, stereoisomer, polymorph, analogue, or pharmaceutically acceptable salt thereof, to a human suffering from a neurobehavioral CNS disorder in which the patient exhibits a central folate deficiency (deficiency of folate level in CNS), wherein the amount of reduced folate is therapeutically effective to improve at least one core symptom of the CNS disorder.

The invention is further directed to a method of treating or ameliorating a symptom associated with ASD in a human child, comprising chronically administering a pharmaceutical composition comprising a dose of folinic acid, or a derivative, active metabolite, prodrug, stereoisomer, polymorph, analogue, or a pharmaceutically acceptable salt of any of the foregoing, to a human child suffering from Autism Spectrum Disorder (ASD), wherein the dose is therapeutically effective to improve at least one core ASD symptom.

The invention is further directed to a method of treating or ameliorating a core symptom of ASD in a subject tested positive for folate receptor-α antibodies (FRAAs) comprising administering to the subject a pharmaceutical composition comprising a dose of a reduced folate (e.g., folinic acid), or a derivative, active metabolite, prodrug, stereoisomer, polymorph, analogue, or a pharmaceutically acceptable salt of any of the foregoing, to the subject. In certain embodiments, the dose is administered without prior ascertaining the CSF level of N(5)-methyl-tetrahydrofolate in the subject, and the administration results in a cessation or improvement of the severity of the symptom. In certain embodiments, the symptom is verbal communication dysfunction, and the treatment results in a statistically significant improvement in the verbal communication of the subject.

In certain preferred embodiments, at least a 2 point increase in verbal communication is achieved within about 12 weeks of administration (e.g., once or twice a day administration) of a reduced folate.

In certain preferred embodiments, the administration of the reduced folate for 12 weeks in accordance with the present invention results in at least a 5-point improvement in a verbal communication score of the subject (e.g., a 5-point improvement, a 6-point improvement, a 7-point improvement, an 8-point improvement, etc.).

The invention is further directed to a method of improving verbal communication in children suffering from Autism Spectrum Disorder (ASD), comprising administering on a chronic basis a therapeutically effective dose of folinic acid, or a derivative, active metabolite, prodrug, stereoisomer, polymorph, analogue, or a pharmaceutically acceptable salt of any of the foregoing, to a human child positive for folate receptor-α antibodies (FRAAs), and assessing the verbal communication of the child before and/or subsequently the first administration, e.g., via an ability-appropriate instrument selected from the CELF-preschool-2, CELF-4 and Preschool Language Scale-5, or a combination of any of the foregoing, in order to determine efficacy of the treatment, wherein an increase in the verbal communication score indicates that treatment is efficacious. In some of these embodiments, at least two, at least three, at least four, or at least five ability-appropriate instruments are used in a sequential order, starting from the most age-appropriate instrument, and continuing with the next lower ability instrument until a score above a floor of the instrument is obtained.

The invention is further directed to a method of improving verbal communication in children suffering from Autism Spectrum Disorder (ASD), comprising assessing whether a child suffering from ASD is positive for folate receptor-α antibodies (FRAAs), assessing the verbal communication of the child who is positive for FRAAs via an ability-appropriate instrument selected from the CELF-preschool-2, CELF-4 and Preschool Language Scale-5, or a combination of any of the foregoing, and administering on a chronic basis a therapeutically effective dose of folinic acid, or a derivative, active metabolite, prodrug, stereoisomer, polymorph, analogue, or a pharmaceutically acceptable salt of any of the foregoing, to the human child if the result of the verbal communication assessment indicates that the child is language impaired, and re-assessing the verbal communication of the child via the ability-appropriate instrument in order to determine efficacy of the treatment.

The invention is also directed in part to a method of orally administering a reduced folate comprising one or more of the following step(s):

(a) preparing a film by the steps of:
(i) combining a polymer, a reduced folate, and water to form a material with a non-self-aggregating uniform heterogeneity;
(ii) forming the material into a film; and
(iii) drying the film in a controlled manner to maintain the non-self-aggregating uniform heterogeneity; and

(b) introducing the film to the oral cavity of a mammal.

In the methods of the present invention, the treatment can be administered on a chronic basis, from disease incubation until disease resolution (if the disease resolves; otherwise the dosing may be continued indefinitely).

In certain embodiments, the reduced folate may be administered in utero to an unborn child suffering from a CNS disorder, and the treatment may be continued after the child is born (if needed). In some of these embodiments, the treatment is initiated after folate receptor-α antibodies (FRAAs) are detected in the serum of the mother of the child.

In certain embodiments, folinic acid, or a derivative, active metabolite, prodrug, stereoisomer, polymorph, analogue, or a pharmaceutically acceptable salt of any of the foregoing is administered in an effective amount to restore and maintain the CSF level of 5-MTHF of from about 40 nmol/L to about 250 nmol/L. In certain embodiments, the patient is 6 months old, or younger, and the treatment restores or maintains the CSF level of 5-MTHF of from about 40 nmol/L to about 240 nmol/L. In certain embodiments, the patient is from about 2.4 months to about 6 months old, and the treatment restores or maintains the CSF level of 5-MTHF of from about 40 nmol/L to about 240 nmol/L. In certain embodiments, the patient is from about 6 months to about 2 years old, and the treatment restores and maintains the CSF level of 5-MTHF of from about 40 nmol/L to about 187 nmol/L. In certain embodiments, the patient is from about 2 years old to about 5 years old, and the treatment restores or maintains the CSF level of 5-MTHF of from about 40 nmol/L to about 150 nmol/L. In certain embodiments, the patient is from about 5 years old to about 10 years old, and the treatment restores or maintains the CSF level of 5-MTHF of from about 40 nmol/L to about 128 nmol/L. In certain embodiments, the patient is from about 10 years old to about 15 years old, and the treatment restores or maintains the CSF level of 5-MTHF of from about 40 nmol/L to about 120 nmol/L. In certain embodiments, the patient is 15 years old, or older, and the treatment restores or maintains the CSF level of 5-MTHF of from about 40 nmol/L to about 120 nmol/L.

In certain preferred embodiments, the reduced folate formulation is administered orally, sublingually, bucally, transdermally, or parenterally, in a dosage form comprising a reduced folate and one or more pharmaceutically acceptable excipient(s). In some of the embodiments, the dosage form may be free from additives and consist of a reduced folate (e.g., folinic acid).

In certain preferred embodiments, the reduced folate (e.g., folinic acid) is administered in a dose from about 0.1 mg/kg/day to about 5 mg/kg/day, or from about 0.5 mg/kg/day to about 4 mg/kg/day, or is administered in a dose from about 1 mg/kg/day to about 3 mg/kg/day, or is administered in a dose from about 1.5 mg/kg/day to about 2.5 mg/kg/day, or is administered in a dose of about 2 mg/kg/day. In certain preferred embodiments, the total dose of reduced folate (e.g., folinic acid) is less than about 500 mg/day, and in certain embodiments less than about 400 mg/day, or less than about 250 mg/day, or about 200 mg or less per day, or about 150 mg or less per day, or about 100 mg or less per day, or about 50 mg or less per day. In certain preferred embodiments, the dose of reduced folate is about 2 mg/kg/day. In certain embodiments wherein the human patient is a child, the reduced folate is administered in a dose from about 0.1 mg/kg/day to about 5 mg/kg/day, or from about 0.5 mg/kg/day to about 4 mg/kg/day, or is administered in a dose from about 1 mg/kg/day to about 3 mg/kg/day. In certain preferred embodiments where the human patient is a child, the total dose of reduced folate (e.g., folinic acid) is less than about 50 mg/day.

The dose of reduced folate described herein is based, e.g., on the D,L folinic acid calcium salt. However, the dose range is applicable to a formulation wherein the reduced folate is the pharmacologically active levo-isomer of d,l-leucovorin or a pharmaceutically acceptable salt thereof (e.g., levoleucovorin calcium), although it is contemplated herein that the dose when administered as levoleucovorin calcium may be reduced, even possibly by half (levoleucovorin injection is dosed at one-half the usual dose of racemic d,l-leucovorin). The dose of reduced folate may be administered in a single dose or in divided daily doses. For example, in certain preferred embodiments, the oral formulation is administered twice daily in divided doses. In some of these embodiments, the folinic acid is levoleucovorin calcium, the doses of levoleucovorin calcium are administered on a twice-a-day basis, and the amount of levoleucovorin calcium administered in each dose is from about 0.25 mg to about 250 mg.

In certain embodiments, the dose of the reduced folate is administered in a route selected from the group consisting of oral, buccal, lingual, sublingual, parenteral, suppository or transdermal, or a combination thereof. In some of these embodiments, the dose of the reduced folate is administered in a route selected from the group consisting of buccal, lingual and sublingual, of a combination thereof.

In some embodiments, the methods of the invention further comprise co-administering a therapeutically effective dose of methylcobalamin with the reduced folate (e.g., folinic acid).

The invention is also directed to a method for identifying an ASD patient that may be responsive to folinic acid treatment comprising the steps of: obtaining a blood sample from an ASD patient; assaying said sample to determine the presence of folate receptor alpha autoantibodies (FRAAs), wherein the presence of FRAAs indicates that the ASD patient may be responsive to folinic acid treatment. In certain preferred embodiments, the method does not include the step of measuring folate concentration in the cerebrospinal fluid.

The invention is further directed to a method for identifying an ASD patient that may be responsive to folinic acid treatment comprising the steps of: obtaining a blood sample from an ASD patient; assaying said sample to determine the glutathione redox ratio and the presence of folate receptor alpha autoantibodies (FRAAs), wherein the presence of FRAAs and a low glutathione redox ratio correlate with an ASD patient responsive to folinic acid treatment. In certain preferred embodiments, the method does not include the step of measuring folate concentration in the cerebrospinal fluid.

In further embodiments, the invention further comprises assessing the verbal communication of the child who is positive for FRAAs via an ability-appropriate instrument selected from the CELF-preschool-2, CELF-4 and Preschool Language Scale-5, or combination of any of the foregoing; administering on a chronic basis a therapeutically effective dose of folinic acid, or a derivative, active metabolite, prodrug, stereoisomer, polymorph, analogue, or a pharmaceutically acceptable salt of any of the foregoing, to the human child if the result of the verbal communication assessment indicates that the child is language impaired, and re-assessing the verbal communication of the child via the ability-appropriate instrument(s) in order to determine efficacy of the treatment.

The invention is further directed to a method for identifying children (e.g., newborns) at risk for developing ASD and/or another CNS disorder comprising the steps of: obtaining a blood sample from a child; and assaying said sample to determine the presence of folate receptor alpha autoantibodies (FRAAs) in the sample, wherein the presence of FRAAs in the sample indicates that the child is at risk of developing ASD and/or another CNS disorder.

The invention is also directed to a method of identifying pregnant female at risk of giving birth to a child with ASD and/or another CNS disorder, the method comprising obtaining a blood sample from the pregnant female; and assaying said sample to determine the presence of folate receptor alpha autoantibodies (FRAAs) in the sample, wherein the presence of FRAAs in the sample indicates that the pregnant female is at risk of giving birth to a child with ASD and/or another CNS disorder and may benefit from administration of a reduced folate.

The invention is also directed to a method of identifying pregnant female at risk of giving birth to a child with Spina Bifida, the method comprising obtaining a blood sample from the pregnant female; and assaying said sample to determine the presence of folate receptor alpha autoantibodies (FRAAs) in the sample, wherein the presence of FRAAs in the sample indicates that the pregnant female is at risk of giving birth to a child with Spina Bifida and may benefit from administration of a reduced folate.

The invention is further directed to a method of reducing a risk of giving birth to a child with ASD in a pregnant female with serum folate receptor alpha autoantibodies (FRAAs), the method comprising administering a dose of reduced folate on a chronic basis to the pregnant female with serum folate receptor alpha autoantibodies (FRAAs).

The invention is also directed to pharmaceutically acceptable formulations comprising a reduced folate and one or more pharmaceutically acceptable excipient(s). The pharmaceutically acceptable formulation may be an oral formulation, a sublingual formulation, a buccal formulation, a transdermal formulation, or an injectable formulation, and may provide an immediate or controlled release of the reduced folate.

The oral formulation may be an oral solid dosage form (e.g., tablet, capsule or powder) or a liquid dosage form (aqueous dispersion, suspension, emulsion, etc.). The oral formulation may also be an orally disintegrating tablet (ODT) or a mini-tablet.

The sublingual formulation may, e.g., be a sublingual film, a sublingual tablet, a sublingual granule, or a sublingual pellet.

The buccal formulation may, e.g., be a lozenge or a gum.

The transdermal formulation may, e.g., be a transdermal patch, a cream, an ointment, or a lotion.

In certain preferred embodiments, the reduced folate is incorporated into an oral dosage form which delivers a therapeutically effective dose of the reduced folate when orally administered to a human. In such embodiments, the oral dosage form may comprise from about 1 mg to about 100 mg of the reduced folate, and may be in the form of orally disintegrating tablet (OTD), mini-tablets, an immediate release tablet, a controlled release tablet, a capsule, sprinkles, or granules. In certain embodiments, the dosage form comprises about 3 mg, about 5 mg, about 7.5 mg, about 10 mg, about 12.5 mg, about 15 mg, about 17.5 mg, about 20 mg, about 22.5 mg, about 25 mg, about 27.5 mg, about 30 mg, about 32.5 mg, about 35 mg, about 37.5 mg, about 40 mg, about 42.5 mg, about 45 mg, about 47.5 mg, or about 50 mg of folinic acid, or an equivalent amount of a different reduced folate.

In certain preferred embodiments, the reduced folate is incorporated into a sublingual dosage form which delivers a therapeutically effective dose of the reduced folate when sublingually administered to a human. The sublingual dosage form may, e.g., be a film or a sublingual tablet.

In certain preferred embodiments, the reduced folate is incorporated into a buccal dosage form which delivers a therapeutically effective dose of the reduced folate when placed in the oral cavity of a human. The sublingual dosage form may, e.g., be a lozenge or a gum.

In certain preferred embodiments, the reduced folate is incorporated into a transdermal dosage form which delivers a therapeutically effective dose of the reduced folate when applied to the skin of the human child. In such embodiments, the reduced folate is incorporated into a transdermal dosage form which delivers, e.g., from about 0.1 mg/kg/day to about 5 mg/kg/day to the human when applied to the skin of the human.

The invention is further directed to a pharmaceutical composition for oral administration, comprising from about 0.25 to about 250 mg folinic acid based on the D,L folinic acid calcium salt, and at least one pharmaceutically acceptable carrier for oral administration.

The invention is further directed to a pharmaceutical composition for oral administration, comprising from about 0.25 to about 250 mg levoleuvorin calcium, and at least one pharmaceutically acceptable carrier for oral administration.

In certain preferred embodiments, the dosage form delivers from about 0.1 mg/kg/day to about 50 mg/kg/day of a reduced folate (e.g., folinic acid) per day.

In some embodiments, the invention is directed in part to a method of testing verbal communication of a subject (e.g., a child) comprising using several instruments together to obtain a final score above a floor level of the instrument. The final score may then be used as the baseline assessment and subsequent assessments of the verbal communication of the subject. In certain embodiments, two, three, four or five different instruments are used. In certain embodiments, the instruments are selected from the group consisting of CELF-preschool-2, CELF-4, Preschool Language Scale-5 (PLS-5), and combinations thereof.

In certain embodiments, the method comprises testing the verbal communication of a subject (e.g., a child) with a first age-appropriate instrument to obtain a first score, and, if the first score is at a floor of the first age-appropriate instrument, testing the verbal communications of the subject with the next lower ability instrument to obtain a second score, and, if the second score is at a floor of the next lower ability instrument, repeating the process until a final score above a floor of the final ability instrument is obtained. The score from the final instrument is then used during as the baseline assessment and subsequent assessments.

### Definitions

As used herein, each of the following terms has the meaning associated with it in this section.

Unless defined otherwise, all technical and scientific terms used herein generally have the same meaning as commonly understood by one of ordinary skill in the art to which this invention belongs. Generally, the nomenclature used herein and the laboratory procedures in biochemistry, analytical chemistry and organic chemistry are those well-known and commonly employed in the art. Standard techniques or modifications thereof are used for chemical syntheses and chemical analyses.

The articles “a” and “an” are used herein to refer to one or to more than one (i.e. to at least one) of the grammatical object of the article. By way of example, “an element” means one element or more than one element.

The term “about” will be understood by persons of ordinary skill in the art and will vary to some extent on the context in which it is used. In the preferred embodiments, the term “about” in the present specification means a value within 20% (±20%) of the value recited immediately after the term “about,” including the value equal to the upper limit (i.e., +20%) and the value equal to the lower limit (i.e., −20%) of this range. For example, the phrase “about 100” encompasses any numeric value that is between 80 and 120, including 80 and 120.

The term “bioequivalent” means the absence of a significant difference in the rate and extent to which the active ingredient or active moiety in pharmaceutical equivalents or pharmaceutical alternatives becomes available at the site of drug action when administered at the same molar dose under similar conditions in an appropriately designed study. Two compositions can be considered as “bioequivalent” if the 90% Confidence Interval of the relative mean Cₘₐₓ and AUC of the test to reference is within 80.00% to 125.00%.

The term “buccal delivery” as used herein means application to and delivery through oral mucosa of a cheek or the mouth cavity.

As used herein, the term “component having a non-neutral pH” includes active ions that, when dissolved in water, give a solution with a pH of less than about 7 (acids) or greater than about 7 (bases). The term is also meant to include compositions of active ions, wherein the composition has a pH of less than about 7 or greater than about 7.

As used herein, the term “component having an acidic pH” and the like is meant to include active ions that, when dissolved in water, give a solution with a pH less than about 7. The term is also meant to include acidic compositions of active ion(s), wherein the composition has a pH of less than about 7.

As used herein, the term “component having a basic pH” is meant to include active ions that, when dissolved in water, give a solution with a pH greater than about 7. The term is also meant to include basic compositions of active ion(s), wherein the composition has a pH of greater than about 7.

“Effective amount” or “therapeutically effective amount” or “equivalent amount” are used interchangeably herein, and refer to an amount of a compound, formulation, material, or composition, as described herein effective to achieve a particular biological result. Such results may include, but are not limited to, the treatment of a disease or condition as determined by any means suitable in the art.

As used herein, the term “child” means a human under the age of 18.

The term “CNS disorder” includes, e.g., ASD, Alzheimer's, Schizophrenia, epilepsy, cerebral folate deficiency (CFD), Attention Deficit Hyperactivity Disorder (“ADHD”), bi-polar disorder, epilepsy, depression, etc.

A “core symptom” includes, e.g., inability to speak, a delay in learning how to speak (after the age of two); speaking in an abnormal tone of voice, or with an odd rhythm or pitch; repeating words or phrases over and over without communicative intent; trouble starting a conversation or keeping it going; difficulty communicating needs or desires; inability to understand simple statements or questions; taking what is said too literally, missing humor, irony, and sarcasm; repetitive body movements (hand flapping, rocking, spinning); obsessive attachment to unusual objects; preoccupation with a narrow topic of interest, sometimes involving numbers or symbols (maps, license plates, sports statistics); a strong need for sameness, order, and routines; clumsiness, abnormal posture, or odd ways of moving; fascination by spinning objects, moving pieces, or parts of toys (e.g. spinning the wheels on a race car, instead of playing with the whole car); hyper- or hypo-reactive to sensory input (e.g. reacts badly to certain sounds or textures, seeming indifference to temperature or pain); unusual or inappropriate body language, gestures, and facial expressions (e.g. avoiding eye contact or using facial expressions that don't match what he or she is saying); lack of interest in other people or in sharing interests or achievements (e.g. showing you a drawing, pointing to a bird); resistance to approach or interact socially with others; difficulty understanding other people's feelings, reactions, and nonverbal cues; resistance to being touched; difficulty or failure to make friends with children the same age. In certain preferred embodiments, the improvement in the core symptom is an improved verbal communication, improved daily living skills, lessened irritability, lessened lethargy, lessened stereotyped behavior, lessened inappropriate speech, reduced hyperactivity, reduce internalization of problems, improved socialization, or a combination of any of the foregoing.

As used herein, the term “lingual delivery” means application to the oral mucosa of the tongue (e.g., on top of the tongue). In preferred embodiments, upon lingual delivery, the reduced folate is rapidly released and is made available for gastrointestinal absorption.

As used herein, the terms “pharmaceutical composition” and “pharmaceutical formulation” and “formulation” are synonymous and refer to a mixture of at least one compound of the invention with other chemical components, such as carriers, stabilizers, diluents, dispersing agents, suspending agents, thickening agents, and/or excipients. The pharmaceutical composition facilitates administration of the compound to an organism. Multiple techniques of administering a compound exist in the art including, but not limited to, intravenous, oral, aerosol, parenteral, ophthalmic, pulmonary and topical administration.

As used herein, “a high dose reduced folate” means 2 mg kg⁻¹ per day of folinic acid up to maximum of 50 mg per day, or an equivalent dose of a different reduced folate.

“Pharmaceutically acceptable” refers to those properties and/or substances that are acceptable to the patient from a pharmacological/toxicological point of view and to the manufacturing pharmaceutical chemist from a physical/chemical point of view regarding composition, formulation, stability, patient acceptance and bioavailability.

“Pharmaceutically acceptable carrier” refers to a medium that does not interfere with the effectiveness of the biological activity of the active ingredient(s) and is not toxic to the host to which it is administered.

The term “subject” refers to an individual who is to be treated.

The term “sublingual delivery”, as used herein, means application to and delivery through oral mucosa underneath the tongue.

As used herein, the term “synerisis” means a process wherein a polymer recoils or separates from the water phase.

The term “treat” or “treating”, as used herein, means reducing the frequency with which symptoms are experienced by a human patient or administering an agent or compound to reduce the frequency and/or severity with which symptoms are experienced. As used herein, “alleviate” or “improve” are used interchangeably with the term “treat.” Treating a disease, disorder or condition may or may not include complete eradication or elimination of the symptom. The term “therapeutic” as used herein means a treatment and/or prophylaxis of a condition or disease state as described herein.

As used herein, “controlled release” or “sustained release” refers to an action or duration of greater than 6 hours (e.g., 8 hours, 10 hours, 12 hours, or 24 hours).

As used herein, “immediate release” means that the formulation releases at least 75% of the reduced folate contained therein within 1 hour.

### BRIEF DESCRIPTION OF THE DRAWINGS

FIG. 1 depicts pathways for folate transport into the brain.

FIG. 2 depicts four interconnected critical folate-related metabolic pathways that manifest abnormalities in individuals with autism spectrum disorder: folate, methylation, glutathione and tetrahydrobiopterin pathways.

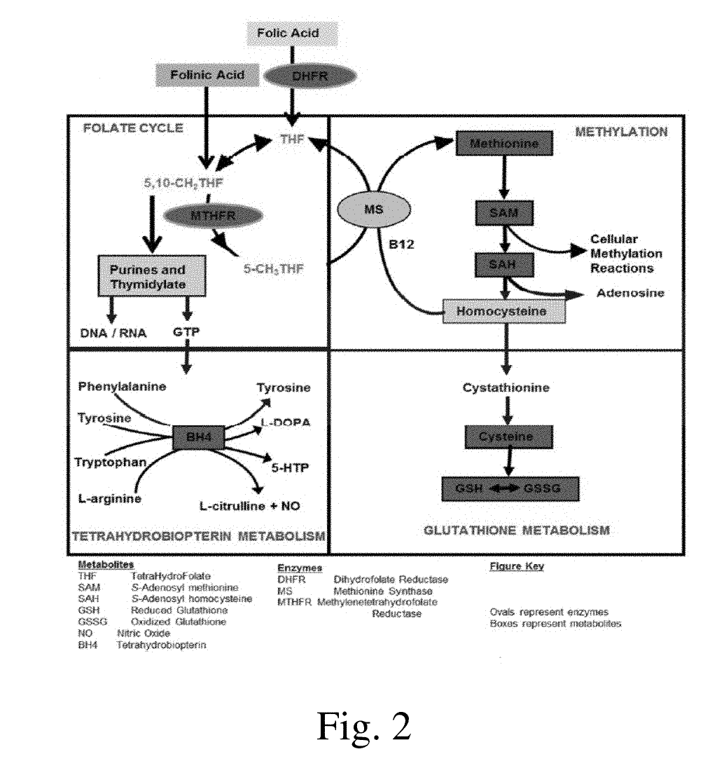

FIG. 3 depicts a flow diagram of participants through the trial described in Example 2.

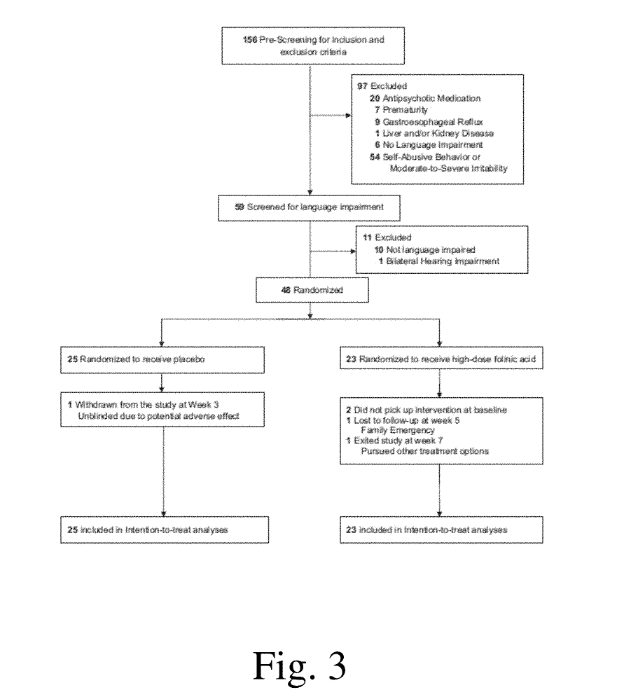

### DETAILED DESCRIPTION

Folate is primarily transported to the CNS across the choroid plexus epithelium attached to the folate receptor α (FRα) using energy-dependent endocytosis. FRα is a high affinity capacity transporter with affinity for folic acid and methylfolate in the nanomolar range.

FRα dysfunction results in a reduced level of folates (e.g., (MTHF)) in the cerebrospinal fluid (CSF) and brain. Initial reports linked FRα dysfunction to the presence of FRα autoantibodies (FRAAs), with later reports also linking FRα dysfunction to mitochondrial disease. Mitochondrial dysfunction and FRAAs block folate binding to adenosine triphosphate (ADP) and block transport of folates into the CNS, leading to reduced levels of folate in the CSF and brain.

Reduced levels of folates (e.g., MTHF) in CSF and brain are implicated in a number of neuro-psychiatric conditions.

In some of these conditions, the folate deficiency is a systemic folate deficiency (a folate deficiency is serum).

In others, despite normal systemic levels, folate transport to the brain is impaired, as, e.g., in the so-called cerebral folate deficiency (CFD) syndromes presenting as developmental and psychiatric disorders. These include infantile-onset CFD syndrome, infantile autism with or without neurologic deficits, a spastic-ataxic syndrome and intractable epilepsy in young children expanding to refractory schizophrenia in adolescents, and finally treatment-resistant major depression in adults. Folate receptor alpha (FRα) autoimmunity with low CSF MTHF underlies most CFD syndromes. Infantile CFD syndrome and autism with neurological deficits also tend to be characterized by elevated FRα antibody titers and low CSF MTHF. Ramaekers, et al., Biochimie. 2016 July; 126:79-90. doi: 10.1016/j.biochi.2016.04.005. Epub 2016 Apr. 8.

The correlation between FRAA and response to reduced folates may be present in many neurological diseases, including epilepsy, CFD, ADHD, bi-polar, depression, etc. and such disease states are considered to be encompassed by the methods of the present invention. The present invention may therefore be used to treat symptoms found in a wide variety of neurobehavioral CNS disorders. Such disorders include, but are not limited to, depression (see, e.g., Pan, et.al, Am J Psychiatry. 2017 Jan. 1; 174(1):42-50. doi: 10.1176/appi.ajp.2016. Ser. No. 15/111,500. Epub 2016 Aug. 13), Alzheimer's disease (AD), schizophrenia (see, e.g., Ramaekers, et al. Mol Genet Metab. 2014 December; 113(4):307-14. doi: 10.1016/j.ymgme.2014.10.002. Epub 2014 Oct. 1), and Autism Spectrum Disorder (ASD).

Described herein are compositions, pharmaceutical compositions, methods for treating, methods for formulating, methods for producing, methods for manufacturing, treatment strategies, pharmacokinetic strategies using reduced folate compounds (e.g., a folinic acid derivative, active metabolite, prodrug, stereoisomer, polymorph, analogue, pharmaceutically acceptable salts of all of the foregoing) for the treatment of at least one symptom (e.g., a core symptom) in a neurobehavioral CNS disorder (e.g., Autism Spectrum Disorder (ASD)).

Prior to the present invention, it did not make sense to increase CNS levels of folates by administering (e.g., orally) folic acid to subjects positive for FRAAs, as the presence of FRAAs indicated a problem with the transport of folic acid into CNS, and it was unexpected that administration of folic acid to the subjects positive for FRAAs would have any effect on language, communication, or other symptoms of ASD (e.g., irritability, socialization, etc.).

Autism Spectrum Disorder

Autism spectrum disorder (ASD) is a behaviorally defined disorder whose etiology remains poorly understood. Recent estimates suggest that up to 2% of children in the United States are affected by an ASD. Recent research has uncovered associated physiological abnormalities, but high-quality clinical trials investigating biological targeted treatments remain limited. Thus, the development and investigation of treatments that target underlying pathophysiological abnormalities and core and associated symptoms is urgently needed.

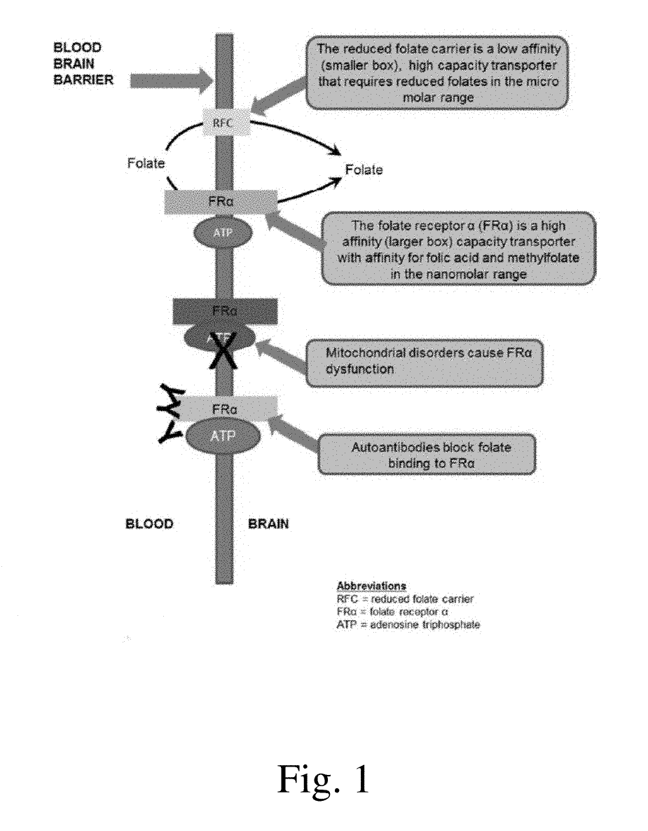

Several abnormalities in the metabolism of folate, an essential water-soluble B vitamin, have been linked to ASD. ASD is associated with polymorphisms in folate-related pathway genes and disruptions in folate-related metabolism may be related to glutathione abnormalities associated with ASD (FIG. 1). Supplementation with folate during the prenatal and conception periods has been shown to lower the risk of ASD in offspring.

Folate is primarily transported across the choroid plexus epithelium attached to the folate receptor α (FRα) using energy-dependent endocytosis (FIG. 1).

Cerebral folate deficiency, a disorder in which folate concentrations are below normal in the cerebrospinal fluid (CSF) but not in the blood, was first described in six children with neurodevelopmental regression and neurological abnormalities. Treatment with folinic acid, a reduced form of folate, normalized CSF folate concentrations and significantly improved neurological symptoms. Further case descriptions demonstrated that many of the children with cerebral folate deficiency had ASD and that treatment with folinic acid improved the ASD symptoms as well as other neurological symptoms. Interestingly, individuals with Rett syndrome, a disorder closely related to ASD, have also been found to have cerebral folate deficiency.

An intriguing finding is that genetic mutations in the FOLR1 gene, which is the gene for the FRα, rarely accounts for cerebral folate deficiency.

Two types of FRAAs, blocking and binding, impair folate transport, and serum titers of the blocking FRAA have been correlated with CSF folate concentrations in independent studies. The blocking FRAA directly interferes with the binding of folate to the FRα while the binding FRAA to the FRα triggers an antibody-mediated immune reaction.

The presence of central folate disturbances in ASD is supported by several studies.

Of 93 children with ASD, 60 and 44% were positive for blocking and binding FRAAs, respectively. Another study which examined only blocking FRAAs in children with ASD confirmed this high prevalence. These rates are clearly higher than the 4-15% prevalence reported in healthy adults and the 3% prevalence reported in developmentally delayed non-autistic children. Interestingly, a recent animal study suggested that FRAAs can disrupt folate metabolism during gestation resulting in ASD-like behaviors in the offspring. More recently, up to 23% of children with ASD who underwent lumbar puncture were reported to have abnormally low CSF folate concentrations.

The reduced folate carrier is a secondary mechanism which transports reduced folates, such as folinic acid, across the blood-brain barrier, although high serum concentrations are required since the reduced folate carrier has a lower affinity for folate (i.e., micromolar concentrations) than the FRα (i.e., nanomolar concentrations; FIG. 1). Case reports and series note that high-dose folinic acid markedly improves symptoms in children with ASD and low CSF folate concentrations.

In one aspect, a novelty of the present application lies, e.g., in a discovery that a compromise or dysfunction of the autoimmunity of folate receptor (as indicated by the presence of FRα autoantibodies (FRAAs)) can be used, independent of gene status, to predict a response to high dose reduced folate medication (e.g., folinic acid) in a subject with ASD.

The methodology described herein not only describes the process for detecting language impairment in autism, but also reports the results of a clinical trial of a high dose reduced folate intervention that validates the claim that the presence of the FRAAs can be sensitive enough to predict which children will have a response to reduced folates in improving verbal communication and other ASD related target symptoms. The uniqueness of this is that, prior to the present invention, it was not-obvious or expected that high doses of reduced folate medication in the presence of FRAAs would have any effect on language, communication or other symptoms related to ADS (e.g., irritability, socialization, etc.).

The use of the presence of FRAAs and their predictive potential for improving ASD symptoms has not been elucidated prior to the present invention.

Discovering the presence of FRAAs early in life may lead to usage of high dosed reduced folates to offset a risk of ASD development in a child.

In certain embodiments, the detection of FRAAs in a pregnant female and subsequent administration of reduced folate may correct the defect in folate and lead to protection of the offspring from neurodegenerative disorders.

The process of measuring the amount of language impairment each child has, as well as the implementation of a battery of validated ASD tools to detect symptom levels and the predicted response to the treatment for those affected described herein has not been fully described in the literature for this population prior to the present invention.

The methodology described herein may be applied to neuropsychiatric conditions other than ASD.

Reduced Folate Drug

In certain embodiments, the reduced folate (drug) is folinic acid, 5-MTHF (5-methyltetrahydrofolate), and derivative, active metabolite, prodrug, stereoisomer, polymorph, analogue, or a pharmaceutically acceptable salt of any of the foregoing. In certain preferred embodiments, the reduced folate is D, L folinic acid calcium salt, or the pharmacologically active levo-isomer of d,l-leucovorin or a pharmaceutically acceptable salt thereof.

In certain embodiments, the reduced folate is leucovorin. Leucovorin is a racemic mixture of the diastereoisomers of the 5-formyl derivative of tetrahydrofolic acid. The biologically active compound of the mixture is the (−)-L-isomer, known as Citrovorum factor, or (−)-folinic acid. Leucovorin does not require reduction by the enzyme dihydrofolate reductase in order to participate in reactions utilizing folates as a source of “one-carbon” moieties. Following oral administration, leucovorin is rapidly absorbed and enters the general body pool of reduced folates. The increase in plasma and serum folate activity (determined microbiologically with Lactobacillus casei) seen after oral administration of leucovorin is predominantly due to 5-methyltetrahydrofolate.

In certain embodiment, the reduced folate comprises a mixture of dextra-leucovorin and levo-leucovorin, wherein levo-leucovorin comprises about 65%, or more, of the mixture by weight. In some of these embodiments, levo-leucovorin comprises from about 70% to about 100% or from about 75% to about 99% or from about 75% to about 95% of the mixture by weight.

In certain preferred embodiments, the drug is levoleucovorin calcium. Levo leucovorin is the levo isomeric form of racemic d,l-leucovorin, present as the calcium salt. Levo leucovorin is the pharmacologically active isomer of leucovorin [(6-S)-leucovorin]. Its molecular weight is 565.6 to 619.6 and its structural formula is:

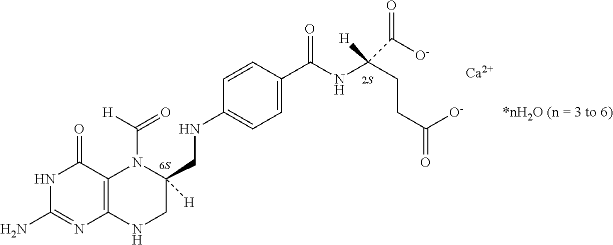 [SMILES: Nc1nc2c(c(=O)[nH]1)N(C=O)[C@@H](CNc1ccc(C(=O)N[C@@H](CCC(=O)[O-])C(=O)[O-])cc1)CN2 ; [Ca+2]]

Levoleucovorin calcium is commercially available in the United States from a variety of manufacturers, as a powder (EQ 50 mg base/vial; EQ 175 mg base/vial; EQ 250 mg base/vial); and as a solution (EQ 175 mg base/vial; EQ 250 mg base/vial). It is used for a variety of indications. For example, it is used with methotrexate to avoid side effects (e.g., in cancer treatment; levoleucovorin injection may ameliorate the hematologic toxicity associated with high-dose methotrexate). It is also used with fluorouracil to help fight colorectal cancer. Levoleucovorin injection is also indicated to diminish the toxicity and counteract the effects of impaired methotrexate elimination and of inadvertent overdosage of folic acid antagonists.

### Dosage Forms

The reduced folate compositions described herein can be formulated for administration to a subject via any conventional means including, but not limited to, oral, parenteral (e.g., intravenous, subcutaneous, or intramuscular), buccal, sublingual, intranasal or transdermal administration routes.

Moreover, the reduced folate compositions described herein can be formulated into any suitable dosage form, including but not limited to, aqueous oral dispersions, aqueous oral suspensions, solid dosage forms including oral solid dosage forms, aerosols, controlled release formulations, fast melt formulations, effervescent formulations, self-emulsifying dispersions, solid solutions, liposomal dispersions, lyophilized formulations, tablets, capsules, pills, powders, delayed release formulations, immediate release formulations, modified release formulations, extended release formulations, pulsatile release formulations, multiparticulate formulations, and mixed immediate release and controlled release formulations.

In some embodiments, the reduced folate formulations provide a therapeutically effective amount of the reduced folate over an interval of about 30 minutes to about 24 hours after administration, enabling, for example, once-a-day, twice-a-day (b.i.d.), or three times a day (t.i.d.) administration if desired.

In one embodiment, the reduced folate composition is formulated into an immediate release oral dosage form (e.g., tablet, capsule, aqueous dispersion, suspension, emulsion, etc.) and is administered on a twice-a-day basis. In other embodiments, the reduced folate is incorporated into a controlled or extended release or pulsatile oral dosage form for administration, e.g., on a once-a-day basis.

In some embodiments, the solid dosage forms of the present invention may be in the form of a tablet, including a suspension tablet, a fast-melt tablet, a bite-disintegration tablet, a rapid-disintegration tablet, an effervescent tablet, or a caplet, a pill, a powder (including a sterile packaged powder, a dispensable powder, or an effervescent powder), a capsule (including both soft or hard capsules, e.g., capsules made from animal-derived gelatin or plant-derived HPMC, or “sprinkle capsules”), solid dispersion, solid solution, bioerodible dosage form, controlled release formulations, pulsatile release dosage forms, multiparticulate dosage forms, pellets, granules, or an aerosol.

In other embodiments, the pharmaceutical formulation is in the form of a powder.

Additionally, pharmaceutical formulations of the present invention may be administered as a single capsule or in multiple capsule dosage form. In some embodiments, the pharmaceutical formulation is administered in two, or three, or four, capsules or tablets.

In some embodiments, solid dosage forms, e.g., tablets, effervescent tablets, and capsules, are prepared by mixing reduced folate particles with one or more pharmaceutical excipient(s) to form a bulk blend composition. When referring to these bulk blend compositions as homogeneous, it is meant that the reduced folate particles are dispersed evenly throughout the composition so that the composition may be readily subdivided into equally effective unit dosage forms, such as tablets, pills, and capsules. The individual unit dosages may also comprise film coatings, which disintegrate upon oral ingestion or upon contact with diluents. These reduced folate formulations can be manufactured by conventional pharmaceutical techniques.

### Preparation of Solid Dosage Forms

Conventional pharmaceutical techniques for preparation of solid dosage forms include, e.g., one or a combination of methods: (1) dry mixing, (2) direct compression, (3) milling, (4) dry or non-aqueous granulation, (5) wet granulation, or (6) fusion. See, e.g., Lachman et al., The Theory and Practice of Industrial Pharmacy (1986). Other methods include, e.g., spray drying, pan coating, melt granulation, granulation, fluidized bed spray drying or coating (e.g., wurster coating), tangential coating, top spraying, tableting, extruding and the like.

### Formulation Components

The pharmaceutical solid dosage forms described herein can comprise the reduced folate compositions described herein and one or more pharmaceutically acceptable additive(s) such as a compatible carrier, binder, complexing agent, ionic dispersion modulator, filling agent, suspending agent, flavoring agent, sweetening agent, disintegrating agent, dispersing agent, surfactant, lubricant, colorant, diluent, solubilizer, moistening agent, plasticizer, stabilizer, penetration enhancer, wetting agent, anti-foaming agent, antioxidant, preservative, or one or more combination thereof. In still other aspects, using standard coating procedures, such as those described in Remington's Pharmaceutical Sciences, 20th Edition (2000), a film coating is provided around the reduced folate formulation. In one embodiment, some or all of the reduced folate particles are coated. In another embodiment, some or all of the reduced folate particles are microencapsulated. In yet another embodiment, some or all of the reduced folate is amorphous material coated and/or microencapsulated with inert excipients. In still another embodiment, the reduced folate particles not microencapsulated and are uncoated.

Suitable carriers for use in the solid dosage forms described herein include, but are not limited to, acacia, gelatin, colloidal silicon dioxide, calcium glycerophosphate, calcium lactate, maltodextrin, glycerin, magnesium silicate, sodium caseinate, soy lecithin, sodium chloride, tricalcium phosphate, dipotassium phosphate, sodium stearoyl lactylate, carrageenan, monoglyceride, diglyceride, pregelatinized starch, hydroxypropylmethylcellulose, hydroxypropylmethylcellulose acetate stearate, sucrose, microcrystalline cellulose, lactose, mannitol and the like.

Suitable filling agents for use in the solid dosage forms described herein include, but are not limited to, lactose, calcium carbonate, calcium phosphate, dibasic calcium phosphate, calcium sulfate, microcrystalline cellulose (e.g., Avicel®, Avicel® PH101, Avicel® PH102, Avicel® PH105, etc.), cellulose powder, dextrose, dextrates, dextran, starches, pregelatinized starch, hydroxypropylmethylcellulose (HPMC), hydroxypropylmethylcellulose phthalate, hydroxypropylmethylcellulose acetate stearate (HPMCAS), sucrose, xylitol, lactitol, mannitol, sorbitol, sodium chloride, polyethylene glycol, and the like.

In order to release the reduced folate from a solid dosage form matrix as efficiently as possible, disintegrants may be used in the formulation, especially when the dosage forms are compressed with binder. Disintegrants help rupturing the dosage form matrix by swelling or capillary action when moisture is absorbed into the dosage form. In some embodiments of the invention, the solid dosage reduced folate formulation has greater than about 1 w % of a disintegrant. In various embodiments of the present invention, the solid dose reduced folate formulations have between about 1 w % to about 11 w % or between about 2 wt % to about 8 wt % disintegrant. In yet other embodiments, the reduced folate formulations have greater than about 2 wt % disintegrant. In some embodiments, combinations of disintegrants provide superior dispersion characteristics compared to single disintegrant at a similar total weight percentage. Suitable disintegrants for use in the solid dosage forms described herein include, but are not limited to, natural starch such as corn starch or potato starch, a pregelatinized starch such as National 1551 or Amijel®, or a sodium starch glycolate such as Promogel® or Explotab®, a cellulose such as a wood product, microcrystalline cellulose, e.g., Avicel®, Avicel® PH101, Avicel® PH102, Avicel® PH105, Elcema® P100, Emcocel®, Vivacel®, Ming Tia®, and Solka-Floc®, methylcellulose, croscarmellose, or a cross-linked cellulose, such as cross-linked sodium carboxymethylcellulose (Ac-Di-Sol®), cross-linked carboxymethylcellulose, or cross-linked croscarmellose, a cross-linked starch such as sodium starch glycolate, a cross-linked polymer such as crosspovidone, a cross-linked polyvinylpyrrolidone, alginate such as alginic acid or a salt of alginic acid such as sodium alginate, a clay such as Veegum® HV (magnesium aluminum silicate), a gum such as agar, guar, locust bean, Karaya, pectin, or tragacanth, sodium starch glycolate, bentonite, a natural sponge, a surfactant, a resin such as a cation-exchange resin, citrus pulp, sodium lauryl sulfate, sodium lauryl sulfate in combination starch, and the like. In one embodiment, Ac-Di-Sol is the disintegrant. The amount of Ac-Di-Sol used in direct compression tableting may vary with typical usage levels between 1 and 3 percent. When added to granulations, generally the same percent is used as with a direct compression formulation. It is often added to both the wet and dried granulations and blends. The amount of Ac-Di-Sol used in capsule formulations generally ranges from 3-6 percent. Reduced interparticle contact within a capsule facilitates the need for elevated levels of disintegrant. Capsules filled on automatic dosater types of equipment, as opposed to semi-automatic or hand-filled machines, are more dense and have a harder structure due to the greater compressional forces needed to form the plug and successfully transfer it into the gelatin or HPMC shell. Greater plug hardness results in greater effectiveness of Ac-Di-Sol.

Binders impart cohesiveness to solid oral dosage form formulations: for powder filled capsule formulation, they aid in plug formation that can be filled into soft or hard shell capsules and in tablet formulation, binders ensure that the tablet remains intact after compression and help assure blend uniformity prior to a compression or fill step. Materials suitable for use as binders in the solid dosage forms described herein include, but are not limited to, carboxymethylcellulose, methylcellulose (e.g., Methocel®), hydroxypropylmethylcellulose (e.g. Hypromellose USP Pharmacoat-603, hydroxypropylmethylcellulose acetate stearate (Aqoate HS-LF and HS), hydroxyethylcellulose, hydroxypropylcellulose (e.g., Klucel®), ethylcellulose (e.g., Ethocel®), and microcrystalline cellulose (e.g., Avicel®), microcrystalline dextrose, amylose, magnesium aluminum silicate, polysaccharide acids, bentonites, gelatin, polyvinylpyrrolidone/vinyl acetate copolymer, crosspovidone, povidone, starch, pregelatinized starch, tragacanth, dextrin, a sugar, such as sucrose (e.g., Dipac®), glucose, dextrose, molasses, mannitol, sorbitol, xylitol (e.g., Xylitab®), lactose, a natural or synthetic gum such as acacia, tragacanth, ghatti gum, mucilage of isapol husks, starch, polyvinylpyrrolidone (e.g., Povidone® CL, Kollidon® CL, Polyplasdone® XL-10, and Povidone® K-12), larch arabogalactan, Veegum®, polyethylene glycol, waxes, sodium alginate, and the like.

In general, binder levels of 20-70% are used in powder-filled gelatin capsule formulations. Binder usage level in tablet formulations is a function of whether direct compression, wet granulation, roller compaction, or usage of other excipients such as fillers which itself can act as moderate binder are used. Formulators skilled in art can determine the binder level for the formulations, but binder usage level of up to 70% in tablet formulations is common.

Suitable lubricants or glidants for use in the solid dosage forms described herein include, but are not limited to, stearic acid, calcium hydroxide, talc, corn starch, sodium stearyl fumarate, alkali-metal and alkaline earth metal salts, such as aluminum, calcium, magnesium, zinc, stearic acid, sodium stearates, magnesium stearate, zinc stearate, waxes, Stearowet®, boric acid, sodium benzoate, sodium acetate, sodium chloride, leucine, a polyethylene glycol or a methoxypolyethylene glycol such as Carbowax™, PEG 4000, PEG 5000, PEG 6000, propylene glycol, sodium oleate, glyceryl behenate, glyceryl palmitostearate, glyceryl benzoate, magnesium or sodium lauryl sulfate, and the like.

Suitable diluents for use in the solid dosage forms described herein include, but are not limited to, sugars (including lactose, sucrose, and dextrose), polysaccharides (including dextrates and maltodextrin), polyols (including mannitol, xylitol, and sorbitol), cyclodextrins and the like.

Non water-soluble diluents are compounds typically used in the formulation of pharmaceuticals, such as calcium phosphate, calcium sulfate, starches, modified starches and microcrystalline cellulose, and microcellulose (e.g., having a density of about 0.45 g/cm3, e.g. Avicel, powdered cellulose), and talc.

Suitable wetting agents for use in the solid dosage forms described herein include, for example, oleic acid, glyceryl monostearate, sorbitan monooleate, sorbitan monolaurate, triethanolamine oleate, polyoxyethylene sorbitan monooleate, polyoxyethylene sorbitan monolaurate, quaternary ammonium compounds (e.g., Polyquat 10®), sodium oleate, sodium lauryl sulfate, magnesium stearate, sodium docusate, triacetin, vitamin E TPGS and the like. Wetting agents include surfactants.

Suitable surfactants for use in the solid dosage forms described herein include, for example, docusate and its pharmaceutically acceptable salts, sodium lauryl sulfate, sorbitan monooleate, polyoxyethylene sorbitan monooleate, polysorbates, polaxomers, bile salts, glyceryl monostearate, copolymers of ethylene oxide and propylene oxide, e.g., Pluronic® (BASF), and the like.

Suitable suspending agents for use in the solid dosage forms described here include, but are not limited to, polyvinylpyrrolidone, e.g., polyvinylpyrrolidone K12, polyvinylpyrrolidone K17, polyvinylpyrrolidone K25, or polyvinylpyrrolidone K30, polyethylene glycol, e.g., the polyethylene glycol can have a molecular weight of about 300 to about 6000, or about 3350 to about 4000, or about 7000 to about 18000, vinyl pyrrolidone/vinyl acetate copolymer (S630), sodium alginate, gums, such as, e.g., gum tragacanth and gum acacia, guar gum, xanthans, including xanthan gum, sugars, cellulosics, such as, e.g., sodium carboxymethylcellulose, methylcellulose, sodium carboxymethylcellulose, hydroxypropylmethylcellulose, hydroxyethylcellulose, polysorbate-80, polyethoxylated sorbitan monolaurate, polyethoxylated sorbitan monolaurate, povidone and the like.

Suitable antioxidants for use in the solid dosage forms described herein include, for example, e.g., butylated hydroxytoluene (BHT), butylhydroxyanisole (BHA), sodium ascorbate, Vitamin E TPGS, ascorbic acid, sorbic acid and tocopherol.

It should be appreciated that there is considerable overlap between additives used in the solid dosage forms described herein. Thus, the above-listed additives should be taken as merely exemplary, and not limiting, of the types of additives that can be included in solid dosage forms of the present invention. The amounts of such additives can be readily determined by one skilled in the art, according to the particular properties desired.

In other embodiments, one or more layers of the pharmaceutical formulation are plasticized. Illustratively, a plasticizer is generally a high boiling point solid or liquid. Suitable plasticizers can be added from about 0.01% to about 50% by weight (w/w) of the coating composition. Plasticizers include, but are not limited to, diethyl phthalate, citrate esters, polyethylene glycol, glycerol, acetylated glycerides, triacetin, polypropylene glycol, polyethylene glycol, triethyl citrate, dibutyl sebacate, stearic acid, stearol, stearate, and castor oil.

### Compressed Tablets

Compressed tablets are solid dosage forms prepared by compacting the bulk blend reduced folate formulations described above. In various embodiments, compressed tablets which are designed to dissolve in the mouth will comprise one or more flavoring agents. In other embodiments, the compressed tablets will comprise a film surrounding the final compressed tablet. In some embodiments, the film coating can provide a delayed release of the reduced folate formulation. In other embodiments, the film coating aids in patient compliance (e.g., Opadry® coatings or sugar coating). Film coatings comprising Opadry® typically range from about 1% to about 3% of the tablet weight. Film coatings for delayed release usually comprise 2-6% of a tablet weight or 7-15% of a spray-layered bead weight. In other embodiments, the compressed tablets comprise one or more excipients.

### Capsule Formulations

A capsule may be prepared, e.g., by placing the bulk blend reduced folate formulation, described above, inside of a capsule. In some embodiments, the reduced folate formulations (non-aqueous suspensions and solutions) are placed in a soft gelatin capsule. In other embodiments, the reduced folate formulations are placed in standard gelatin capsules or non-gelatin capsules such as capsules comprising HPMC. In other embodiments, the reduced folate formulations are placed in a sprinkle capsule, wherein the capsule may be swallowed whole or the capsule may be opened and the contents sprinkled on food prior to eating. In some embodiments of the present invention, the therapeutic dose is split into multiple (e.g., two, three, or four) capsules. In some embodiments, the entire dose of the reduced folate formulation is delivered in a capsule form.

Another useful capsule has a shell comprising the material of the rate-limiting membrane, including any of the coating materials previously discussed, and filled with reduced folate particles. A particular advantage of this configuration is that the capsule may be prepared independently of the reduced folate particles, thus process conditions that would adversely affect the drug can be used to prepare the capsule. A preferred embodiment is a capsule having a shell made of a porous or a pH-sensitive polymer made by a thermal forming process. An especially preferred embodiment is a capsule shell in the form of an asymmetric membrane; i.e., a membrane that has a thin skin on one surface and most of whose thickness is constituted of a highly permeable porous material. A preferred process for preparation of asymmetric membrane capsules comprises a solvent exchange phase inversion, wherein a solution of polymer, coated on a capsule-shaped mold, is induced to phase-separate by exchanging the solvent with a miscible non-solvent. Examples of asymmetric membranes are disclosed in the European Patent Specification 0 357 369 B1.

Yet another useful capsule, a “swelling plug device”, can be used. Reduced folate particles can be incorporated into a non-dissolving capsule-half of the device, which is sealed at one end by a hydrogel plug. This hydrogel plug swells in an aqueous environment, and, after swelling for a predetermined time, exits the capsule thus opening a port through which the reduced folate can leave the capsule and be delivered to the aqueous environment. Preferred hydrogel-plugged capsules are those which exhibit substantially no release of reduced folate from the dosage form until the dosage form has exited the stomach and has resided in the small intestine for about 15 minutes or greater, preferably about 30 minutes or greater, thus assuring that minimal reduced folate is released in the stomach. Hydrogel-plugged capsules of this type have been described in patent application WO90/19168, which is incorporated herein by reference. A reduced folate swelling plug device may be prepared by loading reduced folate into a non-dissolving half-capsule shell which may be formed from a wide variety of materials, including but not limited to polyethylene, polypropylene, poly(methylmethacrylate), polyvinylchloride, polystyrene, polyurethanes, polytetrafluoroethylene, nylons, polyformaldehydes, polyesters, cellulose acetate, and nitrocellulose. The open end of the capsule shell is then “plugged” with a cylindrical plug formed from a hydrogel-forming material, including but not limited to, a homo- or co-poly(alkylene oxide) cross linked by reaction with isocyanate or unsaturated cyclic ether groups, as described in PCT Application WO 90/09168. The composition and length of the hydrogel “plug” is selected to minimize release of reduced folate to the stomach, to decrease the incidence and/or severity of gastrointestinal side effects. The plugged capsule-half is finally sealed with a water-soluble, e.g., gelatin, capsule-half placed over the hydrogel-plugged end of the reduced folate-containing non-dissolving capsule-half. In an embodiment of the “swelling plug device”, the sealed device is coated with a pH-sensitive enteric polymer or polymer mixture, for example cellulose acetate phthalate or copolymers of methacrylic acid and methylmethacrylate. The weight of the enteric polymer coat will generally be from 2 to 20 wt %, preferably from 4 to 15 wt % of the weight of the uncoated sealed capsule. When this preferred “enteric-coated swelling plug device” is ingested orally, the enteric coat prevents release reduced folate in the stomach. The enteric coat dissolves quickly, e.g., within about 15 minutes, in the duodenum, triggering swelling of the hydrogel plug, exiting of the hydrogel plug, and release of the incorporated reduced folate into the gastrointestinal tract at a time greater than about 15 minutes after, and preferably greater than about 30 minutes after, the dosage form has passed from the stomach into the duodenum. Prototype unfilled “swelling plug devices” may be obtained from Scherer DDS Limited, Clydebank, Scotland, under the designation Pulsincap™.

In one embodiment, a reduced folate formulation comprising dried reduced folate particles can be filled in a capsule. An exemplary process for manufacturing the reduced folate particles is the milling/evaporation process. A reduced folate particle suspension comprising 10 to 30 total wt % reduced folate, 1 to 10 total wt % hydroxypropylmethylcellulose (Pharmacoat 603), 0.05 to 0.5 total wt % sodium lauryl sulfate, 0.001 to 0.05 total wt % simethicone emulsion (30% in water), 0.5 to 5% sucrose and 0.1 to 2% NaCl in water is sprayed into a spray granulator using standard parameters known by those skilled in the art. Each wt % is based on the total weight of the suspension. The water is evaporated under vacuum at a temperature of 70 to 90° C. The resulting reduced folate particles comprise about 50 to 80 wt % of reduced folate based on the total weight of the solid particles. Additional excipients such as magnesium stearate, Mannitol and a disintegrant can be added for flow and re-dispersion properties. The particles generally have a median particle size (D50) of about 50 nm to about 1000 nm, more specifically, about 100 nm to about 500 nm. In one embodiment, the capsule is a swelling plug device. In another embodiment, the swelling plug device is further coated with cellulose acetate phthalate or copolymers of methacrylic acid and methylmethacrylate.

In another embodiment, the capsule is a swelling plug device. In another embodiment, the swelling plug device is further coated with cellulose acetate phthalate or copolymers of methacrylic acid and methylmethacrylate.

In yet another embodiment, spray layered reduced folate particles or spray layered reduced folate complex particles are filled in a capsule. An exemplary process for manufacturing the spray layered reduced folate or reduced folate complex particles is the fluidized bed spraying process. Reduced folate suspensions or reduced folate complex suspensions described above are sprayed onto sugar or microcrystalline cellulose (MCC) beads (20-35 mesh) with Wurster column insert at an inlet temperature of 50 to 60° C. and air temp of 30 to 50° C. A 15 to 20 wt % total solids content suspension containing 45 to 80 wt % reduced folate, 10 to 25 wt % hydroxymethylpropylcellulose, 0.25 to 2 wt % of SLS, 10 to 18 wt % of sucrose, 0.01 to 0.3 wt % simethicone emulsion (30% emulsion) and 0.3 to 10% NaCl, based on the total weight of the solid content of the suspension, are sprayed (bottom spray) onto the beads through 1.2 mm nozzles at 10 mL/min and 1.5 bar of pressure until a layering of 400 to 700% wt % is achieved as compared to initial beads weight. The resulting spray layered reduced folate particles or reduced folate complex particles comprise about 30 to 70 wt % of reduced folate based on the total weight of the particles.

The capsule may be pulsatile release reduced folate oral dosage form, comprising: (a) a first dosage unit comprising a first reduced folate dose that is released substantially immediately following oral administration of the dosage form to a patient; (b) a second dosage unit comprising a second reduced folate dose that is released later, e.g., approximately 3 to 15 hours following administration of the dosage form to a patient. In certain embodiments the pulsatile release capsule comprises by weight 30 to 50% of the first reduced folate dose and 50 to 70% of the second reduced folate dose.

### Formulations Containing Coated Reduced Folate Particles

In some embodiments, the spray layered reduced folate particles or spray layered reduced folate complex particles present in reduced folate formulations, such as the capsule formulation described above, is coated. Reduced folate particles can be with a modified release coating, such as an enteric coating using cellulose acetate phthalate or copolymers of methacrylic acid and methylmethacrylate. In one embodiment, the enteric coating may be present in an amount of about 0.5 to 15 wt %, more specifically, about 8 to 12 wt %, based on the weight of the spray layered particles. In one embodiment, the spray layered reduced folate particles or spray layered reduced folate complex particles coated with the enteric coatings can be filled in a modified release capsule in which both enteric coated and immediate release reduced folate beads are filled into a soft gelatin capsule. Additional suitable excipients may also be filled with the coated particles in the capsule.

In another embodiment, mixtures of spray layered reduced folate particles or spray layered reduced folate complex particles coated with the enteric coatings and those without the enteric coatings at appropriate ratios may be encapsulated in a suitable immediate release capsule. The uncoated particles release reduced folate immediately upon administration while the coated particles do not release reduced folate until these particles reach intestine. By controlling the ratios of the coated and uncoated particles, desirable pulsatile release profiles may be obtained. In some embodiments, the ratios between the uncoated and the coated particles are 20/80, or 30/70, or 40/60, or 50/50, w/w to obtain desirable release.

### Tablet Spray Layered Dosage Forms

In some embodiments, the spray layered reduced folate particles or spray layered reduced folate complex particles described above can be compressed into tablets with commonly used pharmaceutical excipients. Any appropriate apparatus for forming the coating can be used to make the enteric coated tablets, e.g., fluidized bed coating using a wurster column, powder layering in coating pans or rotary coaters; dry coating by double compression technique; tablet coating by film coating technique, and the like. See, e.g., U.S. Pat. No. 5,322,655; Remington's Pharmaceutical Sciences Handbook: Chapter 90 “Coating of Pharmaceutical Dosage Forms”, 1990.

In various embodiments, the spray layered reduced folate particles or spray layered reduced folate complex particles described above and one or more excipients are dry blended and compressed into a mass, such as a tablet, having a hardness sufficient to provide a pharmaceutical composition that substantially disintegrates within less than about 30 minutes, less than about 35 minutes, less than about 40 minutes, less than about 45 minutes, less than about 50 minutes, less than about 55 minutes, or less than about 60 minutes, after oral administration, thereby releasing the reduced folate formulation into the gastrointestinal fluid.

In other embodiments, the spray layered reduced folate particles or spray layered reduced folate complex particles with enteric coatings described above and one or more excipients are dry blended and compressed into a mass, such as a tablet. In one embodiment, the enteric coated particles in the tablet substantially avoids release of reduced folate, for example less than 15 wt %, in the stomach but releases substantially all reduced folate (enterically coated), for example, greater than 80 wt %, in the intestine.

In yet other embodiments, a pulsatile release reduced folate formulation comprises a first dosage unit comprising a formulation made from reduced folate containing granules made from a spray drying or spray granulated procedure or a formulation made from reduced folate complex containing granules made from a spray drying or spray granulated procedure without enteric coatings and a second dosage unit comprising spray layered reduced folate particles or spray layered reduced folate complex particles with enteric coatings. In one embodiment, the first dosage unit and the second dosage unit are wet or dry blended and compressed into a mass to make a pulsatile release tablet. In one embodiment, the weight ratio between the uncoated particles and the coated particles is about 1:4 to 4:1.

In another embodiment, binding, lubricating and disintegrating agents are blended (wet or dry) to the spray layered reduced folate or reduced folate complex spray layered beads to make a compressible blend. The first and the second dosage units are compressed separately and then compressed together to form a bilayer tablet.

In yet another embodiment, the first dosage unit is in the form of an overcoat and completely covers the second dosage unit.

Microencapsulated Formulations

In one aspect of the present invention, dosage forms may include microencapsulated reduced folate formulations. In some embodiments, one or more other compatible materials are present in the microencapsulation material. Exemplary materials include, but are not limited to, complexing agents, ionic dispersion modulators, pH modifiers, erosion facilitators, anti-foaming agents, antioxidants, flavoring agents, and carrier materials such as binders, suspending agents, disintegration agents, filling agents, surfactants, solubilizers, stabilizers, lubricants, wetting agents, and diluents.

Materials useful for the microencapsulation described herein include materials compatible with reduced folate which sufficiently isolate reduced folate from other non-compatible excipients. Materials compatible with reduced folate of the present invention are those that delay the release of the reduced folate in vivo.

Exemplary microencapsulation materials useful for delaying the release of the formulations comprising reduced folate include, but are not limited to, hydroxypropyl cellulose ethers (HPC) such as Klucel® or Nisso HPC, low-substituted hydroxypropyl cellulose ethers (L-HPC), hydroxypropyl methyl cellulose ethers (HPMC) such as Seppifilm-LC, Pharmacoat®, Metolose SR, Methocel®-E, Opadry YS, PrimaFlo, Benecel MP824, and Benecel MP843, methylcellulose polymers such as Methocel®-A, hydroxypropylmethylcellulose acetate stearate Aqoat (HF-LS, HF-LG,HF-MS) and Metolose®, Ethylcelluloses (EC) and mixtures thereof such as E461, Ethocel®, Aqualon®-EC, Surelease®, Polyvinyl alcohol (PVA) such as Opadry AMB, hydroxyethylcelluloses such as Natrosol®, carboxymethylcelluloses and salts of carboxymethylcelluloses (CMC) such as Aqualon®-CMC, polyvinyl alcohol and polyethylene glycol co-polymers such as Kollicoat IR®, monoglycerides (Myverol), triglycerides (KLX), polyethylene glycols, modified food starch, acrylic polymers and mixtures of acrylic polymers with cellulose ethers such as Eudragit® EPO, Eudragit® L30D-55, Eudragit® FS 30D Eudragit® L100-55, Eudragit® L100, Eudragit® 5100, Eudragit® RD100, Eudragit® E100, Eudragit® L12.5, Eudragit® 512.5, Eudragit® NE30D, and Eudragit® NE 40D, cellulose acetate phthalate, sepifilms such as mixtures of HPMC and stearic acid, cyclodextrins, parabens, sodium chloride, and mixtures of these materials.

In still other embodiments, plasticizers such as polyethylene glycols, e.g., PEG 300, PEG 400, PEG 600, PEG 1450, PEG 3350, and PEG 800, stearic acid, propylene glycol, oleic acid, and triacetin are incorporated into the microencapsulation material. In other embodiments, the microencapsulating material useful for delaying the release of the pharmaceutical compositions is from the USP or the National Formulary (NF). In yet other embodiments, the microencapsulation material is Klucel. In still other embodiments, the microencapsulation material is methocel.

Microencapsulated reduced folate may be formulated by methods known by one of ordinary skill in the art. Such known methods include, e.g., spray drying processes, spinning disk-solvent processes, hot melt processes, spray chilling methods, spray granulation via fluidized bed, electrostatic deposition, centrifugal extrusion, rotational suspension separation, polymerization at liquid-gas or solid-gas interface, pressure extrusion, or spraying solvent extraction bath. In addition to these, several chemical techniques, e.g., complex coacervation, solvent evaporation, polymer-polymer incompatibility, interfacial polymerization in liquid media, in situ polymerization, in-liquid drying, and desolvation in liquid media could also be used. Furthermore, other methods such as roller compaction, extrusion/spheronization, coacervation, or nanoparticle coating may also be used.

The spinning disk method allows for: 1) an increased production rate due to higher feed rates and use of higher solids loading in feed solution, 2) the production of more spherical particles, 3) the production of a more even coating, and 4) limited clogging of the spray nozzle during the process.

Spray granulation via a fluid bed is often more readily available for scale-up. In various embodiments, the material used in the spray-granulation encapsulation process is emulsified or dispersed into the core material in a concentrated form, e.g., 10-60% solids. The microencapsulation material is, in one embodiment, emulsified until about 1 to 3 μm droplets are obtained. Once a dispersion of reduced folate and encapsulation material is obtained, the emulsion is fed as droplets into the heated chamber of the spray granulator. In some embodiments, the droplets are sprayed into the chamber or spun off a rotating disk. The microspheres are then dried in the heated chamber and fall to the bottom of the chamber where they are harvested.

Roller compaction, which involves dry granulation of single powder or a blended mixture of powders by the use of pressure to form dense compacts (the compacts are subsequently milled to a desired particle size), provides another alternative. It is a simple process that is readily available for use, and does not involved the use of solvents for granulation. Thus, roller compaction eliminates the exposure of sensitive active pharmaceutical ingredients to moisture and drying. Roller compaction can also provide some enhanced stability and taste-masking characteristics to active pharmaceutical by diluting and isolating such components in a granulated matrix of compatible ingredients. Roller compaction also imparts increased density and flow to the powder.

Extrusion/spheronization is another method that involves wet massing of active pharmaceutical ingredients, followed by the extrusion of the wet mass through a perforated plate to produce short cylindrical rods. These rods are subsequently placed into a rapidly rotating spheronizer to shape the cylindrical rods into uniform spheres. The spheres are subsequently dried using a fluid bed drier and then coated with a functional coating using a fluid bed equipped with a Wurster insert and spray nozzle.

Coacervation involves microencapsulation of materials such as active pharmaceutical ingredients and involves a three part process of particle or droplet formation, coacerate wall formation, and capsule isolation. This method can produce very small particle size microcapsules (10-70 microns).

In one embodiment, the reduced folate particles are microencapsulated prior to being formulated into one of the above forms. In still another embodiment, some or most of the reduced folate particles are coated prior to being further formulated by using standard coating procedures, such as those described in Remington's Pharmaceutical Sciences, 20th Edition (2000).

Coated or Plasticized Formulations

In other embodiments, the solid dosage reduced folate formulations are plasticized (coated) with one or more layers. Illustratively, a plasticizer is generally a high boiling point solid or liquid. Suitable plasticizers can be added from about 0.01% to about 50% by weight (w/w) of the coating composition. Plasticizers include, but are not limited to, diethyl phthalate, citrate esters, polyethylene glycol, glycerol, acetylated glycerides, triacetin, polypropylene glycol, polyethylene glycol, triethyl citrate, dibutyl sebacate, stearic acid, stearol, stearate, and castor oil.

In other embodiments a powder comprising the reduced folate formulations described herein may be formulated to comprise one or more pharmaceutical excipients and flavors. Such a powder may be prepared, for example, by mixing the reduced folate formulation and optional pharmaceutical excipients to form a bulk blend composition. Additional embodiments also comprise a suspending agent and/or a wetting agent. This bulk blend is uniformly subdivided into unit dosage packaging or multi-dosage packaging units. The term “uniform” means the homogeneity of the bulk blend is substantially maintained during the packaging process. In some embodiments, at least about 75% to about 85% of the reduced folate has an effective particle size by weight of less than 500 nm to about 100 nm. In other embodiments, the reduced folate comprises at least 90% reduced folate particles having an effective particle size by weight of less than 500 nm to about 100 nm.

Powders

In certain embodiments of the invention, the reduced folate is incorporated into a dosage form comprising a pharmaceutically acceptable powder. The powder may be reconstituted at the time of use, e.g., via admixing with water for injection for a parenteral dosage form, or with a wide variety of liquids as described herein to prepare an oral dosage form. In still other embodiments, effervescent powders are also prepared in accordance with the present invention. Effervescent salts have been used to disperse medicines in water for oral administration. Effervescent salts are granules or coarse powders containing a medicinal agent in a dry mixture, usually composed of sodium bicarbonate, citric acid and/or tartaric acid. When salts of the present invention are added to water, the acids and the base react to liberate carbon dioxide gas, thereby causing “effervescence.” Examples of effervescent salts include, e.g.: sodium bicarbonate or a mixture of sodium bicarbonate and sodium carbonate, citric acid and/or tartaric acid. Any acid-base combination that results in the liberation of carbon dioxide can be used in place of the combination of sodium bicarbonate and citric and tartaric acids, as long as the ingredients were suitable for pharmaceutical use and result in a pH of about 6.0 or higher.

The method of preparation of the effervescent granules of the present invention employs three basic processes: wet granulation, dry granulation and fusion. The fusion method is used for the preparation of most commercial effervescent powders. It should be noted that, although these methods are intended for the preparation of granules, the formulations of effervescent salts of the present invention could also be prepared as tablets, according to known technology for tablet preparation.

Wet and Dry Granulation

Wet granulation is one of the oldest methods of granule preparation. The individual steps in the wet granulation process of tablet preparation include milling and sieving of the ingredients, dry powder mixing, wet massing, granulation, drying and final grinding. In various embodiments, the reduced folate composition is added to the other excipients of the pharmaceutical formulation after they have been wet granulated.

Dry granulation involves compressing a powder mixture into a rough tablet or “slug” on a heavy-duty rotary tablet press. The slugs are then broken up into granular particles by a grinding operation, usually by passage through an oscillation granulator. The individual steps include mixing of the powders, compressing (slugging) and grinding (slug reduction or granulation). No wet binder or moisture is involved in any of the steps. In some embodiments, the reduced folate formulation is dry granulated with other excipients in the pharmaceutical formulation. In other embodiments, the reduced folate formulation is added to other excipients of the pharmaceutical formulation after they have been dry granulated.

### Solid Dispersions

In other embodiments, the reduced folate formulations described herein are solid dispersions. Methods of producing such solid dispersions are known in the art and include, but are not limited to, for example, U.S. Pat. Nos. 4,343,789, 5,340,591, 5,456,923, 5,700,485, 5,723,269, and U.S. Pub. Appl 2004/0013734, each of which is specifically incorporated by reference. In some embodiments, the solid dispersions of the invention comprise both amorphous and non-amorphous reduced folate and can have enhanced bioavailability as compared to conventional reduced folate formulations. In still other embodiments, the reduced folate formulations described herein are solid solutions. Solid solutions incorporate a substance together with the active agent and other excipients such that heating the mixture results in dissolution of the drug and the resulting composition is then cooled to provide a solid blend which can be further formulated or directly added to a capsule or compressed into a tablet. Methods of producing such solid solutions are known in the art and include, but are not limited to, for example, U.S. Pat. Nos. 4,151,273, 5,281,420, and 6,083,518, each of which is specifically incorporated by reference.

Modified Release Forms, Including Controlled Release and Delayed Release

The pharmaceutical solid oral dosage forms comprising the reduced folate formulations described herein can be further formulated to provide a modified or controlled release of reduced folate.

In some embodiments, the solid dosage forms described herein can be formulated as a delay release dosage form such as and enteric coated delayed release oral dosage forms, i.e., as an oral dosage form of a pharmaceutical composition as described herein which utilizes an enteric coating to affect release in the small intestine of the gastrointestinal tract. The enteric coated dosage form may be a compressed or molded or extruded tablet/mold (coated or uncoated) containing granules, powder, pellets, beads or particles of the active ingredient and/or other composition components, which are themselves coated or uncoated. The enteric coated oral dosage form may also be a capsule (coated or uncoated) containing pellets, beads or granules of the solid carrier or the composition, which are themselves coated or uncoated. Enteric coatings may also be used to prepare other controlled release dosage forms including extended release and pulsatile release dosage forms.

In other embodiments, the reduced folate formulations described herein are delivered using a pulsatile dosage form. Pulsatile dosage forms comprising the reduced folate formulations described herein may be administered using a variety of formulations known in the art. For example, such formulations include, but are not limited to, those described in U.S. Pat. Nos. 5,011,692, 5,017,381, 5,229,135, and 5,840,329, each of which is specifically incorporated by reference. Other dosage forms suitable for use with the reduced folate formulations are described in, for example, U.S. Pat. Nos. 4,871,549, 5,260,068, 5,260,069, 5,508,040, 5,567,441 and 5,837,284, all of which are specifically incorporated by reference. In one embodiment, the controlled release dosage form is pulsatile release solid oral dosage form comprising at least two groups of particles, each containing the reduced folate formulation described herein. The first group of particles provides a substantially immediate dose of reduced folate upon ingestion by a subject. The first group of particles can be either uncoated or comprise a coating and/or sealant. The second group of particles comprises coated particles, which comprise from about 2% to about 75%, preferably from about 2.5% to about 70%, and more preferably from about 40% to about 70%, by weight of the total dose of the reduced folate in said formulation, in admixture with one or more binders. The coating comprises a pharmaceutically acceptable ingredient in an amount sufficient to provide a delay of from about 2 hours to about 7 hours following ingestion before release of the second dose. Suitable coatings include one or more differentially degradable coatings such as, by way of example only, pH sensitive coatings (enteric coatings) such as acrylic resins (e.g., Eudragit® EPO, Eudragit® L30D-55, Eudragit® FS 30D Eudragit® L100-55, Eudragit® L100, Eudragit® 5100, Eudragit® RD100, Eudragit® E100, Eudragit® L12.5, Eudragit® 512.5, and Eudragit® NE30D, Eudragit® NE 40D®) either alone or blended with cellulose derivatives, e.g., ethylcellulose, or non-enteric coatings having variable thickness to provide differential release of the reduced folate formulation.

Many other types of controlled release systems known to those of ordinary skill in the art and are suitable for use with the reduced folate formulations described herein. Examples of such delivery systems include, e.g., polymer-based systems, such as polylactic and polyglycolic acid, plyanhydrides and polycaprolactone; porous matrices, nonpolymer-based systems that are lipids, including sterols, such as cholesterol, cholesterol esters and fatty acids, or neutral fats, such as mono-, di- and triglycerides; hydrogel release systems; silastic systems; peptide-based systems; wax coatings, bioerodible dosage forms, compressed tablets using conventional binders and the like. See, e.g., Liberman et al., Pharmaceutical Dosage Forms, 2 Ed., Vol. 1, pp. 209-214 (1990); Singh et al., Encyclopedia of Pharmaceutical Technology, 2nd Ed., pp. 751-753 (2002); U.S. Pat. Nos. 4,327,725, 4,624,848, 4,968,509, 5,461,140, 5,456,923, 5,516,527, 5,622,721, 5,686,105, 5,700,410, 5,977,175, 6,465,014 and 6,932,983, each of which is specifically incorporated by reference.

In another embodiment, a modified release dosage formulation may comprise a combination of: (a) a compressed tablet core comprising a poorly water soluble active agent, a pharmaceutically acceptable water swellable polymer, and an osmotic agent; and (b) an outer coating layer which completely covers the tablet core and comprises a pH sensitive coating. An optional sealing coat may be applied to the compressed tablet core and an optional coating layer comprising an enteric coating agent may be applied under the outer coating layer as an inner coating or as an overcoat over the outer coating layer. The tablet core may be compressed using a smooth faced tablet die. In one embodiment, the active agent is a reduced folate.

The osmotic agent in this dosage form is any non-toxic pharmaceutically acceptable water soluble compound which will dissolve sufficiently in water and increase the osmotic pressure inside the tablet core. Suitable osmotic agents include simple sugars and salts such as sodium chloride, potassium chloride, magnesium sulfate, magnesium sulfate, magnesium chloride, sodium sulfate, lithium sulfate, urea, inositol, sucrose, lactose, glucose, sorbitol, fructose, mannitol, dextrose, magnesium succinate, potassium acid phosphate and the like. The preferred osmotic agent for the tablet core is a simple sugar such as anhydrous lactose in the range of 0-50% by weight, based on the weight of the compressed, uncoated tablet.

The water swellable polymer may be any pharmaceutically acceptable polymer which swells and expands in the presence of water to slowly release reduced folate. These polymers include polyethylene oxide, methylcellulose, hydroxypropylcellulose, hydroxypropylmethylcellulose and the like. In a preferred embodiment, the water swellable polymer will be polyethylene oxide (obtained from Union Carbide Corporation under the trade name Polyox WSR Coagulant or Polyox WSR N 80). These materials form a viscous gel in water or other solvent system at a sufficient concentration to control the release of the reduced folate. This will generally require a concentration of the pharmaceutically acceptable, water swellable polymer of about 0-50% by weight of the compressed, uncoated tablet.

The outer coating comprises a pH sensitive coating which functions as an enteric polymer in that it does not begin to dissolve until pH conditions in excess of the pH of the stomach region are encountered. The pH sensitive coating is the same type of material that is described above. The pH sensitive coating may be present in an amount of about 0.5-15 wt %, more specifically, about 8-12 wt %, based on the weight of the coated tablet core.

Certain controlled release formulation may release less than about 20 wt % of reduced folate in the formulation is released within the first three hours after administration and more than about 60 percent of reduced folate between 3 and 10 hours. Other controlled release reduced folate formulation may release less than about 50 percent within the first three hours after administration and about 50 percent of reduced folate between 3 and 15 hours.

Enteric Coatings

Enteric coatings should be applied to a sufficient thickness such that the entire coating does not appreciably dissolve in the gastrointestinal fluids at pH below about 5 after 1 hour, but does dissolve at pH about 5 and above. It is expected that any anionic polymer exhibiting a pH-dependent solubility profile can be used as an enteric coating in the practice of the present invention to achieve delivery to the lower gastrointestinal tract. In some embodiments the polymers for use in the present invention are anionic carboxylic polymers. In other embodiments, the polymers and compatible mixtures thereof, and some of their properties, include, but are not limited to:

Shellac, also called purified shellac, a refined product obtained from the resinous secretion of an insect. This coating dissolves in media of pH>7;

### Acrylic Polymers

The performance of acrylic polymers (primarily their solubility in biological fluids) can vary based on the degree and type of substitution. Examples of suitable acrylic polymers include methacrylic acid copolymers and ammonia methacrylate copolymers. The Eudragit series E, L, S, RL, RS and NE (Rohm Pharma) are available as solubilized in organic solvent, aqueous dispersion, or dry powders. The Eudragit series RL, NE, and RS are insoluble in the gastrointestinal tract but are permeable and are used primarily for colonic targeting. The Eudragit series E dissolve in the stomach. The Eudragit series L, L-30D and S are insoluble in stomach and dissolve in the intestine; Opadry Enteric are also insoluble in stomach and dissolve in the intestine.

### Cellulose Derivatives

Examples of suitable cellulose derivatives are: ethyl cellulose; reaction mixtures of partial acetate esters of cellulose with phthalic anhydride. The performance can vary based on the degree and type of substitution. Cellulose acetate phthalate (CAP) dissolves in pH>6. Aquateric (FMC) is an aqueous based system and is a spray dried CAP psuedolatex with particles<1 μm. Other components in Aquateric can include pluronics, Tweens, and acetylated monoglycerides. Other suitable cellulose derivatives include: cellulose acetate trimellitate (Eastman); methylcellulose (Pharmacoat, Methocel); hydroxypropylmethyl cellulose phthalate (HPMCP); hydroxypropylmethyl cellulose succinate (HPMCS); and hydroxypropylmethylcellulose acetate succinate (e.g., AQOAT (Shin Etsu)). The performance can vary based on the degree and type of substitution. For example, HPMCP such as, HP-50, HP-55, HP-555, HP-55F grades are suitable. The performance can vary based on the degree and type of substitution. For example, suitable grades of hydroxypropylmethylcellulose acetate succinate include, but are not limited to, AS-LG (LF), which dissolves at pH 5, AS-MG (Mf), which dissolves at pH 5.5, and AS-HG (HF), which dissolves at higher pH. These polymers are offered as granules, or as fine powders for aqueous dispersions; PolyVinyl Acetate Phthalate (PVAP). PVAP dissolves in pH>5 and it is much less permeable to water vapor and gastric fluids.

In some embodiments, the coating can, and usually does, contain a plasticizer and possibly other coating excipients such as colorants, talc, and/or magnesium stearate, which are well known in the art. Suitable plasticizers include triethyl citrate (Citroflex 2), triacetin (glyceryl triacetate), acetyl triethyl citrate (Citroflec A2), Carbowax 400 (polyethylene glycol 400), diethyl phthalate, tributyl citrate, acetylated monoglycerides, glycerol, fatty acid esters, propylene glycol, and dibutyl phthalate. In particular, anionic carboxylic acrylic polymers usually will contain 10-25% by weight of a plasticizer, especially dibutyl phthalate, polyethylene glycol, triethyl citrate and triacetin. Conventional coating techniques such as spray or pan coating are employed to apply coatings. The coating thickness must be sufficient to ensure that the oral dosage form remains intact until the desired site of topical delivery in the intestinal tract is reached.

Colorants, detackifiers, surfactants, antifoaming agents, lubricants (e.g., carnuba wax or PEG) may be added to the coatings besides plasticizers to solubilize or disperse the coating material, and to improve coating performance and the coated product.

A particularly suitable methacrylic copolymer is Eudragit L®, particularly L-30D® and Eudragit 100-55®, manufactured by Rohm Pharma, Germany. In Eudragit L-30D®, the ratio of free carboxyl groups to ester groups is approximately 1:1. Further, the copolymer is known to be insoluble in gastrointestinal fluids having pH below 5.5, generally 1.5-5.5, i.e., the pH generally present in the fluid of the upper gastrointestinal tract, but readily soluble or partially soluble at pH above 5.5, i.e., the pH values present in the small intestine.

In some embodiments, materials include shellac, acrylic polymers, cellulosic derivatives, polyvinyl acetate phthalate, and mixtures thereof. In other embodiments, materials include Eudragit® series E, L, RL, RS, NE, L, L300, S, 100-55, cellulose acetate phthalate, Aquateric, cellulose acetate trimellitate, ethyl cellulose, hydroxypropylmethylcellulose phthalate, hydroxypropylmethylcellulose acetate succinate, polyvinyl acetate phthalate, and Cotteric.

Mini-Tablets

In some embodiments, pharmaceutical reduced folate formulations are provided as mini-tablets. Mini-tablets are tablets which are smaller than conventional tablets but larger than conventional powder particles in a conventional capsule. Mini-tablets typically provide more surface area than conventional tablets, and, in certain embodiments, may provide for ability to adjust/control the release rate of reduced folates more easily.

Each mini-tablet is generally from about 2 to about 4 mm in diameter. Conventional powder particles put in a conventional capsule are about from about 100 to about 600 microns in diameter. A plurality of mini-tablets is usually placed in a gelatin capsule.

Mini-tablets generally provide an immediate release of the reduced folate. However, mini-tablets may also provide a sustained release of the reduced folate.

In certain embodiments, the mini-tablet comprises a reduced folate and a pharmaceutically acceptable diluent.

### Films

In some embodiments, pharmaceutical reduced folate formulations are provided as films for administration into oral cavity of a human, e.g., edible, digestable, or dissolvable films.

In some embodiments, upon placement into the oral cavity (e.g., application to the oral mucosa) of a human, the film begins to dissolve based on the compositional profile created during formulation and the reduced folate (e.g., levo leucovorin) and release the reduced folate at a controlled or immediate release rate sufficient to achieve a therapeutically effective concentration of the reduced folate in serum and/or CNS (e.g., CSF). In some of these embodiments, the film provides Tmax of the reduced folate of from about 5 minutes to about 4 hours, from about 10 minutes to about 3 hours, from about 20 minutes to about 3 hours, from about 30 minutes to about 2.5 hours, from about 30 minutes to about 2 hours, from about 45 minutes to about 2 hours, from about 1 hour to about 2 hours, or from about 30 minutes to about 1.5 hours.

In some embodiments, films of the invention are formulated to provide buccal delivery of a reduced folate, e.g., through transcellular and/or intercellular transport across the buccal mucosa. In some of the preferred embodiments, buccal delivery provides controlled release of the reduced folate to optimize absorption and/or improved bioavailability.

In some embodiments, films of the invention are formulated to provide sublingual delivery of a reduced folate. In some of these embodiments, the sublingual delivery provides rapid disintegration of the film and release the reduced folate to sublingual mucosa. For example, in some of the embodiments, the film provides Tmax of the reduced folate that is shorter than the Tmax obtained after oral administration of an equivalent amount of the reduced folate. For example, in some of the embodiments, after sublingual administration, the film provides Tmax of from about 1 minute to about 1 hours, from about 2 minutes to about 55 minutes, from about 3 minutes to about 50 minutes, from about 4 minutes to about 45 minutes, from about 5 minutes to about 45 minutes, from about 5 minutes to about 40 minutes, from about 10 minutes to about 35 minutes, from about 10 minutes to about 30 minutes, or from about 10 minutes to about 20 minutes.

In some embodiments, films of the invention are formulated to provide lingual delivery of a reduced folate. In some of these embodiments, lingual delivery provides rapid disintegration and releases the reduced folate to allow subsequent absorption in the gastrointestinal tract. In some of these embodiments, the films of the invention are bioequivalent to oral formulation (e.g., solid oral dosage forms) comprising an equivalent dose of the reduced folate.

In some embodiments, films of the invention are formulated to provide buccal, sublingual and lingual delivery of a reduced folate, and can be administered buccally, sublingually and lingually.

In some embodiments, the films of the invention are capable for matching a pharmacokinetic profile of an oral dosage form, but with a more convenient dosing for children with ASD and/or another other CNS disorder.

In some of the embodiments, films of the present invention are dissolvable in the presence of liquid, such as in the oral cavity of the human or when mixed with a liquid, such as water.

Dissolvable films of the invention generally fall into three main classes: fast dissolving, moderate dissolving and slow dissolving. Fast dissolving films generally dissolve in about 1 second to about 30 seconds. Moderate dissolving films generally dissolve in about 1 to about 30 minutes, and slow dissolving films generally dissolve in more than 30 minutes, e.g., up to about 60 minutes or more. Fast dissolving films may comprise low molecular weight hydrophilic polymers (i.e., polymers having a weight average molecular weight between about 1,000 to 200,000). In contrast, slow dissolving films generally comprise high weight average molecular weight polymers (i.e., having a weight average molecular weight in the millions).

In certain embodiments, the film is substantially dissolvable within a short time of time (e.g., within 30 seconds to 5 minutes) when exposed to water, alcohol or aqueous mixture of alcohols.

In certain embodiments, a pharmaceutically acceptable polymer is used as a film former to hold a reduced folate with or without additional pharmaceutically acceptable excipients in place. The film former generally comprises a pharmaceutically acceptable polymer and/or a blend of several pharmaceutically acceptable polymers. The blend may, for example, comprise four, three, or two pharmaceutically acceptable polymers. In certain embodiments, the film may further comprise a pH modifier and/or a permeation enhancer to achieve target absorption.

The film-forming polymer may, e.g., be water soluble, water insoluble, or comprise a combination of one or more either water soluble or water insoluble polymers. The polymer may include cellulose or a cellulose derivative. Specific examples of useful water soluble polymers include, e.g., pullulan, hydroxypropylmethyl cellulose, hydroxyethyl cellulose, hydroxypropyl cellulose, polyvinyl pyrrolidone, carboxymethyl cellulose, polyvinyl alcohol, sodium aginate, polyethylene glycol, xanthan gum, tragancanth gum, guar gum, acacia gum, arabic gum, polyacrylic acid, methylmethacrylate copolymer, carboxyvinyl copolymers, starch, and combinations thereof. Specific examples of useful water insoluble polymers include, but are not limited to, ethyl cellulose, hydroxypropyl ethyl cellulose, cellulose acetate phthalate, hydroxypropyl methyl cellulose phthalate and combinations thereof.

Other polymers useful for incorporation into the films of the present invention include biodegradable polymers, copolymers, block polymers and combinations thereof. Among the known useful polymers or polymer classes which meet the above criteria are: poly(glycolic acid) (PGA), poly(lactic acid) (PLA), polydioxanoes, polyoxalates, poly(α-esters), polyanhydrides, polyacetates, polycaprolactones, poly(orthoesters), polyamino acids, polyaminocarbonates, polyurethanes, polycarbonates, polyamides, poly(alkyl cyanoacrylates), and mixtures and copolymers thereof. Additional useful polymers include, stereopolymers of L- and D-lactic acid, copolymers of bis(p-carboxyphenoxy) propane acid and sebacic acid, sebacic acid copolymers, copolymers of caprolactone, poly(lactic acid)/poly(glycolic acid)/polyethyleneglycol copolymers, copolymers of polyurethane and poly(lactic acid), copolymers of polyurethane and poly(lactic acid), copolymers of α-amino acids, copolymers of α-amino acids and caproic acid, copolymers of α-benzyl glutamate and polyethylene glycol, copolymers of succinate and poly(glycols), polyphosphazene, polyhydroxy-alkanoates and mixtures thereof. Binary and ternary systems are contemplated.

Other specific polymers that may be used in the films of the invention include, e.g., those marketed under the Medisorb and Biodel trademarks. The Medisorb materials are marketed by the Dupont Company of Wilmington, Del. and are generically identified as a “lactide/glycolide co-polymer” containing “propanoic acid, 2-hydroxy-polymer with hydroxy-polymer with hydroxyacetic acid.” Four such polymers include lactide/glycolide 100 L, believed to be 100% lactide having a melting point within the range of 338°−347° F. (170°−175° C.); lactide/glycolide 100 L, believed to be 100% glycolide having a melting point within the range of 437°−455° F. (225°−235° C.); lactide/glycolide 85/15, believed to be 85% lactide and 15% glycolide with a melting point within the range of 338°−347° F. (170°−175° C.); and lactide/glycolide 50/50, believed to be a copolymer of 50% lactide and 50% glycolide with a melting point within the range of 338°−347° F. (170°−175° C.).

The Biodel materials represent a family of various polyanhydrides which differ chemically.

In certain embodiments, the film-forming polymer comprises polyethylene oxide or a blend of polyethylene oxides of different weight average molecular weights. In some of these embodiments, polyethylene oxide or a blend of different polyethylene oxides is blended with one or more cellulosic polymer(s).

In certain embodiments, the film comprises at least one water-soluble polymer containing from about 20% to about 100% by weight polyethylene oxide, from about 0% to about 80% by weight hydroxypropylmethyl cellulose, and from about 1% to about 80% a reduced folate. In some of these embodiments, the film is free of added plasticizers, surfactants, and polyalcohols.

In some embodiments, polyethylene oxide comprises from about 20% to 100% by weight of the polymer component. In some embodiments, the amount of polyethylene oxide ranges from about 1 mg to about 200 mg. The hydrophilic cellulosic polymer ranges from about 0% to about 80% by weight, or in a ratio of up to about 4:1 with polyethylene oxide, or in a ratio of about 1:1 with polyethylene oxide.

In certain embodiments, the film of the invention comprises a reduced folate uniformly dispersed throughout a film. The uniform distribution allows, e.g. large area films to be initially formed, and subsequently cut into individual dosage units without concern for whether each unit is compositionally equal. In some of the embodiments, the concentration of the reduced folate throughout the film does not vary by more than about 20%, about 15%, about 10%, or about 5% per unit area of the film. The reduced uniform distribution may, e.g., be achieved by controlling one or more parameters, and particularly the elimination of air pockets prior to and during film formation and the use of a drying process that reduces aggregation or conglomeration of the components in the film as it forms into a solid structure.

In some embodiments, the film further comprises a pH modifier and/or a permeation enhancer and/or a stabilizer. A variety of pH modifiers and/or permeation enhancers and/or stabilizers may be used to achieve target absorption.

In some embodiments, the film comprises a pH modulated polymer system that reduces or prevents synerisis when combined in water with components having a non-neutral pH, such as active ions. Active ions, such as acids, bases or buffer systems, may be used to achieve delivery of a drug contained in the same film or a different film at a desired pH.

In some embodiments, the edible film comprises (i) an edible water soluble polymer; (ii) an anti-tacking agent comprising a lubricant, an anti-adherent, a glidant or any combination thereof; and (iii) a reduced folate.

The edible water soluble polymer may comprise from about 1% to about 95% of the edible film by weight and, in certain embodiments, is selected from the group, consisting of hydroxypropyl cellulose, hydroxypropylmethyl cellulose, polydextrose, polyethylene oxide and combinations thereof.

The anti-tacking agent may, e.g., comprise from about 0.01% to about 30% of the edible film by weight.

The reduced folate may, e.g., be folinic acid or the levo isomer of folinic acid, and may comprise from about 5% to about 95% of the formulation by weight.

In some embodiments, the film comprises reduced folate particles dispersed in a water soluble polymer composition including polyethylene oxide and a saccharide-based polymer.

A variety of other components and fillers may also be added to the films of the present invention. Each additional component may comprise up to about 80%, or from about 3% to about 50%, or from about 3% to about 20% based on the weight of the film. These may include, without limitation, surfactants; plasticizers which assist in compatibilizing the components within the mixture; polyalcohols; anti-foaming agents, such as silicone-containing compounds, which promote a smoother film surface by releasing oxygen from the film; and thermo-setting gels such as pectin, carageenan, and gelatin, which help in maintaining the dispersion of components.

The films of the invention may further comprise lubricants, buffering agents, stabilizers, blowing agents, pigments, coloring agents, fillers, bulking agents, sweetening agents, flavoring agents, fragrances, release modifiers, adjuvants, plasticizers, flow accelerators, mold release agents, polyols, granulating agents, diluents, binders, buffers, absorbents, glidants, adhesives, anti-adherents, acidulants, softeners, resins, demulcents, solvents, surfactants, emulsifiers, elastomers and mixtures thereof.

The films may, e.g., further include, gelatin, vegetable proteins such as sunflower protein, soybean proteins, cotton seed proteins, peanut proteins, grape seed proteins, whey proteins, whey protein isolates, blood proteins, egg proteins, acrylated proteins, water-soluble polysaccharides such as alginates, carrageenans, guar gum, agar-agar, xanthan gum, gellan gum, gum arabic and related gums (gum ghatti, gum karaya, gum tragancanth), pectin, water-soluble derivatives of cellulose: alkylcelluloses hydroxyalkylcelluloses and hydroxyalkylalkylcelluloses, such as methylcelulose, hydroxymethylcellulose, hydroxyethylcellulose, hydroxypropylcellulose, hydroxyethylmethylcellulose, hydroxypropylmethylcellulose, hydroxybutylmethylcellulose, cellulose esters and hydroxyalkylcellulose esters such as cellulose acetate phthalate (CAP), hydroxypropylmethylcellulose (HPMC); carboxyalkylcelluloses, carboxyalkylalkylcelluloses, carboxyalkylcellulose esters such as carboxymethylcellulose and their alkali metal salts; water-soluble synthetic polymers such as polyacrylic acids and polyacrylic acid esters, polymethacrylic acids and polymethacrylic acid esters, polyvinyl acetates, polyvinylalcohols, polyvinylacetatephthalates (PVAP), polyvinylpyrrolidone (PVP), PVY/vinyl acetate copolymer, and polycrotonic acids; also suitable are phthalated gelatin, gelatin succinate, crosslinked gelatin, shellac, water soluble chemical derivatives of starch, cationically modified acrylates and methacrylates possessing, for example, a tertiary or quaternary amino group, such as the diethylaminoethyl group, which may be quaternized if desired; and other similar polymers.

The films may further include inorganic fillers, such as the oxides of magnesium aluminum, silicon, titanium, etc. desirably in a concentration range of about 0.02% to about 3% by weight and desirably about 0.02% to about 1% based on the weight of all components.

Further examples of additives are plasticizers which may be included in the films are polyalkylene oxides, such as polyethylene glycols, polypropylene glycols, polyethylene-propylene glycols, organic plasticizers with low molecular weights, such as glycerol, glycerol monoacetate, diacetate or triacetate, triacetin, polysorbate, cetyl alcohol, propylene glycol, sorbitol, sodium diethylsulfosuccinate, triethyl citrate, tributyl citrate, and the like. These additives may be added in concentrations ranging from about 0.5% to about 30%, or from about 0.5% to about 20% based on the weight of the polymer.

The films may further include compounds to improve the flow properties of the starch material such as animal or vegetable fats, desirably in their hydrogenated form, especially those which are solid at room temperature. These fats desirably have a melting point of 50° C. or higher. Preferred are tri-glycerides with C₁₂-, C₁₄-, C₁₆-, C₁₈-, C₂₀- and C₂₂-fatty acids. These fats can be added alone without adding extenders or plasticizers and can be advantageously added alone or together with mono- and/or di-glycerides or phosphatides, especially lecithin. The mono and di-glycerides are desirably derived from the types of fats described above, i.e. with C₁₂-, C₁₄-, C₁₆-, C₁₈-, C₂₀- and C₂₂-fatty acids.

The total amounts used of the fats, mono-, di-glycerides and/or lecithins are up to about 5% and preferably within the range of about 0.5% to about 2% by weight of the total composition

It is further useful to add silicon dioxide, calcium silicate, or titanium dioxide in a concentration of about 0.02% to about 1% by weight of the total composition. These compounds act as texturizing agents.

In certain embodiments, films of the invention may include lecithin. Lecithin can be included in an amount of from about 0.1% to about 10% or from about 0.25% to about 2.00% by weight. Other surface active agents, i.e. surfactants, include, but are not limited to, cetyl alcohol, sodium lauryl sulfate, the Spans™ and Tweens™ which are commercially available from ICI Americas, Inc. Ethoxylated oils, including ethoxylated castor oils, such as Cremophor® EL which is commercially available from BASF, are also useful. Carbowax™ is yet another modifier which is very useful in the present invention. Tweens™ or combinations of surface active agents may be used to achieve the desired hydrophilic-lipophilic balance (“HLB”) The present invention, however, does not require the use of a surfactant and films or film-forming compositions of the present invention may be essentially free of a surfactant while still providing the desirable uniformity features of the present invention.

In certain embodiments, it may be further useful to add polydextrose to the films of the present invention. Polydextrose serves as a filler and solubility enhancer, i.e., it increases the dissolution time of the films in the oral cavity.

As additional modifiers which enhance the procedure and product of the present invention are identified, Applicants intend to include all such additional modifiers within the scope of the invention claimed herein.

Non-limiting examples of binders include, e.g., starches, pregelatinize starches, gelatin, polyvinylpyrrolidone, methylcellulose, sodium carboxymethylcellulose, ethylcellulose, polyacrylamides, polyvinyloxoazolidone, and polyvinylalcohols.

In certain embodiments, the film may comprise, by weight, from about 0.1% to about 80% reduced folate, from about 2% to about 5% polyethylene oxide, from about 2% to about 5% hydroxypropyl cellulose, from about 0.1% to about 1% polydextrose, from about 0.1% to about 1% sucralose, from about 0.01% to about 0.5% titanium dioxide, and from about 1% to about 5% flavoring agent.

In certain embodiments, the film may comprise, by weight, from about 0.1% to about 80% reduced folate, from about 0% to about 70% hydroxypropyl methylcellulose, from about 0% to about 40% hydroxypropyl cellulose, from about 0% to about 40% pectin, from about 0% to about 30% artificial flavors, from about 0.01% to about 30% polydextrose, from about 0% to about 10% sodium carboxymethylcellulose, from about 0% to about 20% erythritol, from about 0.01% to about 5% sucralose, from about 0% to about 10% citric acid, from about 0.01% to about 10% magnesium stearate, from about 0% to about 1% glyceryl monooleate, from about 0.01% to about 2% silica, from about 0% to about 1% polysorbate, from about 0% to about 1% sorbitan monooleate, from about 0% to about 1% potassium sorbate, from about 0% to about 0.1% sodium benzoate, from about 0% to about 10% sodium hexametaphosphate, from about 0% to about 25% propylene glycol, from about 0% to about 10% gum arabic from, and from about 0.01% to about 1% dye.

In certain embodiments, the invention is directed to an oral film having reduced adherence to oral cavity comprising a reduced folate and a water-soluble polymer as an essential polymer base in combination with a film former, with the weight ratio of water-soluble polymer to the film former from about 25:1 to about 250:1. In some of these embodiments, the oral film comprises a reduced folate, a water-soluble polymer as an essential polymer base in combination with pectin as a film former, wherein the ratio of water-soluble polymer to the pectin is about 250:1 to about 25:1.

The edible films of the invention may have a thickness of from about 500 μm to about 1,500 or from about 20 mils to about 60 mils, and when dried have a thickness from about 3 μm to about 250 or about 0.1 mils to about 10 mils. In the preferred embodiments, the dried films will have a thickness of about 2 mils to about 8 mils, of from about 3 mils to about 6 mils. In some of the embodiments, the thickness of the film is uniform.

In certain embodiments, oral films of the invention are bioequivalent to conventional immediate release oral dosage forms and exhibit one or more favorable properties such as fast dissolution time, improved drug loading, improved mechanical properties (burst strength, tensile strength, modules of elongation).

A variety of different film-forming techniques may be used, including methods that will provide a flexible film, e.g., a reverse roll coating. The flexibility of the film allows for the sheets of film to be rolled and transported for storage or prior to being cut into individual dosage forms. In the preferred embodiments, the films are self-supporting (i.e., the films are able to maintain their integrity and structure in the absence of a separate support).

In certain embodiments, the films are formed by a controlled drying process, or other process that maintains the required uniformity of the film. The films of the invention, may, e.g., be prepared by a process comprising the steps of combining a polymer component, polar solvent and an active component to form a matrix with a uniform distribution of the components; forming a film from the matrix; providing a surface having top and bottom sides; feeding the film onto the top side of the surface; and drying the film by applying heat to the bottom side of the surface and exposing the film to a temperature above a degradation temperature of the active component, wherein the active component is maintained at the desired level.

The films of the invention may also be formed by a process comprising the steps of: combining a polymer, a polar solvent and an active component to form a material with a non-self-aggregating uniform heterogeneity; forming the material into a film; and drying the film at a temperature above a degradation temperature of the active component, wherein the active component is maintained at the desired level.

Coating or casting methods are also useful for the purpose of forming the films of the invention. Specific examples include reverse roll coating, gravure coating, immersion or dip coating, metering rod or meyer bar coating, slot die or extrusion coating, gap or knife over roll coating, air knife coating, curtain coating, or combinations thereof, especially when a multi-layered film is desired.

The gravure coating process relies on an engraved roller running in a coating bath, which fills the engraved dots or lines of the roller with the coating material. The excess coating on the roller is wiped off by a doctor blade and the coating is then deposited onto the substrate as it passes between the engraved roller and a pressure roller.

In certain embodiments, the films are formed by utilizing a selected casting or deposition method and a controlled drying process. Examples of controlled drying processes include, but are not limited to, the use of the apparatus disclosed in U.S. Pat. No. 4,631,837 to Magoon (“Magoon”), herein incorporated by reference, as well as hot air impingement across the bottom substrate and bottom heating plates. Another drying technique for obtaining the films of the present invention is controlled radiation drying, in the absence of uncontrolled air currents, such as infrared and radio frequency radiation (i.e. microwaves).

In certain embodiments, the films are formed into a sheet prior to drying. For example, in certain embodiments, after the desired components are combined to form a multi-component matrix, including the polymer, water, and an active or other components as desired, the combination is formed into a sheet or film, by any method known in the art such as extrusion, coating, spreading, casting or drawing the multi-component matrix. If a multi-layered film is desired, this may be accomplished by co-extruding more than one combination of components which may be of the same or different composition. A multi-layered film may also be achieved by coating, spreading, or casting a combination onto an already formed film layer.

In certain embodiments, the film is formed by a method comprising the steps of providing a polymeric matrix, forming a small-scale form of a reduced folate, dispersing the small-scale form of the reduced folate throughout the polymeric matrix, and drying the polymeric matrix so as to form a self-supporting film dosage composition including the small-scale form of the active agent. The small-scale form of the reduced folate may be in the form of a microparticle or a nanoparticle. The small-scale form of the reduced folate may be formed through emulsion processing, through milling, and/or through a microfluidics pumping apparatus. In some embodiments, the small-scale form of the reduced folate may be formed via a high shear apparatus. The small-scale form of the reduced folate may also be formed in situ, or may be added as a preformed small-scale form. In cases where the small-scale form of the active is formed in situ, the process of forming the film may be used to stabilize the small-scale form of the reduced folate and thus prevent agglomeration. The small-scale form of the reduced folat may be bonded to one or more ligands.

In certain embodiments, the reduced folates are be incorporated into the film compositions of the present invention such that the film composition provides a controlled release form of the reduced folate for a period of time. For example, particles of a drug may be coated with polymers, such as ethyl cellulose or polymethacrylate, which are commercially available under brand names such as Aquacoat ECD and Eudragit E-100, respectively. Solutions of a drug may also be absorbed on such polymer materials and incorporated into the inventive film compositions. Other components may also be employed in such controlled release compositions.

Each of the films of the present invention may be divided into smaller individual film units which may be sized and packaged to provide dosage units for consumption.

In certain embodiments, the films of the invention release an effective amount of a reduced folate to restore and maintain the CSF level of 5-MTHF of from about 40 nmol/L to about 250 nmol/L in a subject after administration of the film to the subject once or twice a day for a time period of from 1 week to 54 weeks, of from 1 week to 28 weeks, of from 2 weeks to 14 weeks.

Film formulations described herein may be administered using a variety of films which have been described in the art. For example, such films include, but are not limited to, U.S. Pat. Nos. 7,357,891; 7,425,292; 7,666,337; 7,910,641; 8,017,150; 8,241,661; 8,282,954; 8,900,497; 9,108,340; 9,150,341; 9,303,918; 9,511,033; 10,034,833; and U.S. Patent Publications Nos. 2007/0122455; 2008/0044454; 2007/0104270; 2012/0076921; 2012/0100202; 2014/0008831; 2016/0175199; 2018/0140559; 2018/0280518; 2018/0200198; each of which is specifically incorporated by reference in its entirety.

### Orally Dispersable Tablet (ODT)

In some embodiments, pharmaceutical reduced folate formulations are provided as orally dispersable tablets (“ODTs”), which disintegrate in the oral cavity of a subject upon contact with the subject's saliva (e.g., within about 3 minutes, about 2 minutes, or about 1 minute of contact with the saliva). In certain embodiments, ODTs disintegrate substantially within about 30 seconds. The disintegration of the ODTs in the oral cavity of the subject ideally provides a smooth, easy-to-swallow suspension having no gritty mouthfeel or aftertaste.

In certain embodiments, ODT dosage form comprises a compressed matrix comprising a reduced folate, a disintegrant and a sugar alcohol. The ODT dosage form may also include pharmaceutically acceptable excipients typically used in disintegrating tablet formulations such as microcrystalline cellulose and spray dried mannitol (compressible diluents), croscarmellose sodium or crospovidone (super disintegrant), coloring agents, and optionally magnesium stearate or sodium stearyl fumarate (lubricant intragranularly mixed or used externally to lubricate die and punch surfaces).

In certain embodiments, ODT dosage form comprises a compressed granulation comprising (i) rapidly dispersing microgranules and (ii) granules comprising a therapeutically effective amount of a reduced folate. Rapidly dispersing microgranules can be prepared as described, e.g., in US Publication Nos. 2006/0078614, 2006/0105038, 2005/0232988 or 2003/0215500 (each of which is herein incorporated by reference in its entirety for all purposes) by granulating a disintegrant with a sugar alcohol and/or saccharide having an average particle size of not more than about 30 μm. The granulation can be carried out, for example, in a high shear granulator with approximately 20-25% water as the granulating fluid, and if needed wet milled and dried to produce rapidly dispersing microgranules, for example having an average particle size of not more than about 300 μm (e.g., about 175-300 μm).

In certain embodiments, rapidly dispersing microgranules comprise a sugar alcohol such as mannitol and/or a saccharide such as lactose and a disintegrant such as Crospovidone. The sugar alcohol and/or saccharide and disintegrant will typically be present in the rapidly dispersing microgranules at a ratio of from about 99:1 to about 90:10 (sugar alcohol and/or saccharide:disintegrant). For example, the rapidly dispersing microgranules used in the ODT formulations disclosed in the various examples in accordance with the present invention may be produced by granulating 95 parts of D-mannitol with an average particle size of about 15 μm, and 5 parts of crospovidone (Crospovidone XL-10) in a high shear mixer (e.g., GMX 600 from Vector Corporation) with water as the granulating fluid, drying the wet mass in a fluid bed dryer (e.g., Glatt GPCG 200 or Fluid Air FA0300), and sieving/milling to obtain granules with an average particle size of less than 400 μm. Alternately, the wet milled granules are dried in a tray drying oven for a loss on drying value of less than 1% by weight.

In certain embodiments, rapidly dispersing microgranules may be produced, e.g., by granulating a disintegrant such as Crospovidone XL-10 with a sugar alcohol or a saccharide, or a combination thereof, each having an average particle diameter of not more than about 30 μm, with water or an alcohol-water mixture in a conventional or high shear granulator and drying in a fluid bed equipment or a tray drying oven to produce granules with an average particle size not more than about 400 μm (preferably not more than about 300 μm).

The ratio of the disintegrant to the sugar alcohol, saccharide, or mixture thereof in the ODT or rapidly dispersing microgranules generally ranges from about 90/10 to about 99/01. In certain embodiments, the ratio is about 90/10, about 91/9, about 92/8, about 93/7, about 94/6, about 95/5, about 96/4, about 97/3, about 98/2, about 99/1, inclusive of all values, ranges, and subranges in between.

The ratio of the rapidly dispersing microgranules to particles comprising reduced folate may range, e.g., from about 5/1 to about 1/1, including about 5/1, 4/1, 3/1, 2/1, 1/1, inclusive of all values, ranges, and subranges in between.

The average particle size of the particles containing a therapeutically effective dose of reduced folate is not more than about 400 μm, or in some embodiments not more than about 300 μm.

ODT dosage forms, comprising the pharmaceutical composition of the present invention have a low friability, e.g., less than about 1%, (e.g., less than about 0.9%, less than about 0.8%, less than about 0.7%, less than about 0.6%, less than about 0.5%, less than about 0.4%, less than about 0.3%, etc., inclusive of all ranges and subranges in between) in order to have sufficient durability to withstand handling, shipping, and/or packaging in push-through blister packaging.

A non-limiting list of suitable disintegrants for the rapidly dispersing microgranules includes crospovidone (cross-linked PVP), sodium starch glycolate, cross-linked sodium carboxymethylcellulose, calcium silicate, and low substituted hydroxypropyl cellulose. The amount of disintegrant in the ODT is typically in the range of about 1% to about 10% by weight, including about 1%, about 2%, about 3%, about 4%, about 5%, about 6%, about 7%, about 8%, about 9%, or about 10%, inclusive of all ranges and subranges therebetween. In a particular embodiment, the disintegrant for the rapidly dispersing microgranules is selected from the group consisting of crospovidone, cross-linked sodium carboxymethylcellulose, and low substituted hydroxypropyl cellulose. In a more particular embodiment, the disintegrant for the rapidly dispersing microgranules is crospovidone.

A non-limiting list of suitable sugar alcohols includes mannitol, sorbitol, xylitol, maltitol, arabitol, ribitol, dulcitol, iditol, isomalt, lactitol, erythritol and combinations thereof. In a particular embodiment, the sugar alcohol is mannitol. A non-limiting list of suitable saccharides includes lactose, sucrose, maltose, and combinations thereof. In a particular embodiment, the saccharide is lactose. The amount of sugar alcohol and/or saccharide in the ODT ranges from about 30% to about 70% by weight, including, for example, about 30%, about 35%, about 40%, about 45%, about 50%, about 55%, about 60%, about 65%, or about 70%, inclusive of all ranges and subranges in between.

Pharmaceutically acceptable excipients include fillers, diluents, glidants, disintegrants, binders, lubricants etc. Other pharmaceutically acceptable excipients include acidifying agents, alkalizing agents, preservatives, antioxidants, buffering agents, chelating agents, coloring agents, complexing agents, emulsifying and/or solubilizing agents, flavors and perfumes, humectants, sweetening agents, wetting agents etc. Examples of suitable fillers, diluents and/or binders include lactose (e.g. spray-dried lactose, α-lactose, β-lactose, Tabletose®, various grades of Pharmatose®, Microtose® or Fast-Flo®), microcrystalline cellulose (various grades of Avicel®, Ceolus®, Elcema®, Vivacel®, Ming Tai® or Solka-Floc®), hydroxypropylcellulose, L-hydroxypropylcellulose (low substituted), low molecular weight hydroxypropyl methylcellulose (HPMC) (e.g. Methocel E, F and K from Dow Chemical, Metolose SH from Shin-Etsu, Ltd), hydroxyethylcellulose, sodium carboxymethylcellulose, carboxymethylhydroxyethyl cellulose and other cellulose derivatives, sucrose, agarose, sorbitol, mannitol, dextrins, maltodextrins, starches or modified starches (including potato starch, maize starch and rice starch), calcium phosphate (e.g. basic calcium phosphate, calcium hydrogen phosphate, dicalcium phosphate hydrate), calcium sulfate, calcium carbonate, sodium alginate, collagen etc.

Examples of suitable diluents include e.g. calcium carbonate, dibasic calcium phosphate, tribasic calcium phosphate, calcium sulfate, microcrystalline cellulose, powdered cellulose, dextrans, dextrin, dextrose, fructose, kaolin, lactose, mannitol, sorbitol, starch, pregelatinized starch, sucrose, sugar etc.

Examples of suitable disintegrants include e.g. alginic acid or alginates, microcrystalline cellulose, hydroxypropyl cellulose and other cellulose derivatives, croscarmellose sodium, crospovidone, polacrillin potassium, sodium starch glycolate, starch, pregelatinized starch, carboxymethyl starch (e.g. Primogel® and Explotab®) etc.

Specific examples of glidants and lubricants include stearic acid, magnesium stearate, calcium stearate or other metallic stearates, talc, waxes and glycerides, light mineral oil, PEG, glyceryl behenate, colloidal silica, hydrogenated vegetable oils, corn starch, sodium stearyl fumarate, polyethylene glycols, alkyl sulfates, sodium benzoate, sodium acetate etc.

Other excipients include e.g. flavoring agents, coloring agents, taste-masking agents, pH-adjusting agents, buffering agents, preservatives, stabilizing agents, anti-oxidants, wetting agents, humidity-adjusting agents, surface-active agents, suspending agents, absorption enhancing agents, agents for modified release etc.

Other excipients include e.g. flavoring agents, coloring agents, taste-masking agents, pH-adjusting agents, buffering agents, preservatives, stabilizing agents, anti-oxidants, wetting agents, humidity-adjusting agents, surface-active agents, suspending agents, absorption enhancing agents, agents for modified release etc.

In certain embodiments, the rapidly dispersing microgranules and taste-masked drug-containing microparticles may be present in the ratio of about 4/1 to 2/1 to achieve a smooth mouthfeel.

In certain embodiments, ODT dosage forms are prepared by a method comprising:
(a) preparing rapidly dispersing microgranules comprising a disintegrant and a sugar alcohol, a saccharide or a mixture thereof, wherein each of the disintegrant, sugar alcohol and/or saccharide have an average particle diameter of not more than 30 μm; t
(b) preparing microgranules comprising reduced folate and optionally one or more pharmaceutically acceptable excipient(s), e.g., in a mixer or V-blender; and
(c) compressing the blend of rapidly dispersing microgranules and the microgranules comprising reduced folate into an ODT, e.g., using an externally lubricated tablet press to provide ODTs with desired tableting characteristics (e.g., adequate hardness, friability of <0.6%, low disintegration time, and rapid dissolution).

### Sublingual Tablets

In some embodiments, pharmaceutical reduced folate formulations are provided as sublingual tablets. Sublingual tablets are small and flat, for placement under the tongue and designed for rapid, almost instantaneous disintegration and release of drug to the sublingual mucosa.

In some of the preferred embodiments, sublingual tablets combine transmucosal delivery (e.g., avoid first-pass metabolism, or provide rapid delivery into circulation) with lingual delivery to enhance one or more pharmacokinetic parameters (e.g., Tmax, Cmax, AUC, etc.). Permeation enhancers can optionally be used to promote transmucosal delivery.

The sublingual tablets of the present invention disintegrate, to release the reduced folate for rapid absorption under the tongue, within five minutes and, more preferably, within a two minute or one minute period of time.

In certain embodiments, a sublingual tablet according to the invention comprises a reduced folate, a disintegrant and a taste masking flavoring agent. The flavoring agent may comprise from about 5% to about 90% of the sublingual tablet by weight. In certain embodiments, the diluent may comprise from about 15% to about 35% of the sublingual tablet by weight.

In certain embodiments, a sublingual tablet comprises a core comprising a reduced folate, a disintegrant and a taste masking flavoring agent.

In certain embodiments, the disintegrant is mannitol, microcrystalline cellulose, sodium glycolate, or a mixture of any of the foregoing.

In certain embodiments, the taste masking flavoring agent comprises mannitol, sodium saccharine, peppermint, spearmint, magnasweet, vanilla, or a mixture of any of the foregoing materials.

In certain embodiments, the sublingual tablet further comprises a lubricant (e.g., a magnesium stearate).

In certain embodiments, a sublingual tablet comprises the following components in parts by weight: from about 0.1 to about 20% reduced folate, from about 70 to about 90% mannitol, from about 10 to about 13% pregelatinized starch, from about 0.3 to about 0.8% sweetener.

The sublingual tablets may be prepared by blending the components in a suitable blender to form a homogeneous mixture and compressing the homogeneous mixture in a tablet press to form a sublingual tablet having the desired release characteristics.

The sublingual tablets are administered by placing a single sublingual tablet under the tongue. The tablet is allowed to disintegrate and release the reduced folate. The released reduced folate is then absorbed by the sublingual mucosa.

Lozenges

In some embodiments, pharmaceutical reduced folate formulations are provided as lozenges that are dissolvable in the oral cavity of a human (e.g., within 2-10 minutes of being placed into the oral cavity of the human).

In some of the preferred embodiments, lozenges are designed to release a reduced folate buccally.

In certain embodiments, lozenges combine buccal delivery with transmucosal delivery (e.g., avoid first-pass metabolism, or provide rapid delivery into circulation) with lingual delivery to enhance one or more pharmacokinetic parameters (e.g., Tmax, Cmax, AUC, etc.). Permeation enhancers can optionally be used to promote transmucosal delivery.

A lozenge may comprise a reduced folate, an absorbent excipient (e.g., mannitol or β-cyclodextrin) and a sweetener (e.g., xylitol, optionally in combination with ammonium glycyrrhizinate).

In certain embodiments, the reduced folate may be dispersed in an absorbent excipient. The absorbent excipient may comprise, e.g., mannitol; cyclodextrins, including α-, β-, and γ-cyclodextrin, as well as derivatives of cyclodextrins, such as trimethyl-β-cyclodextrin, dimethyl-β-cyclodextrin, hydroxyethyl-β-cyclodextrin, and hydroxypropyl-β-cyclodextrin; silica preparations, such as the synthetic silica formulation marketed under the trade name Syloid™ by W. R. Grace Limited of North Dale House, North Circular Road, London; cellulosic materials, such as Avicel microcellulose manufactured by FMC Corporation; and other conventional binders and fillers used in the food industry, such as acacia powder, gelatin, gum arabic, and sorbitol.

The absorbent excipient is typically present in an amount between about 5 and 25% by weight (wt %), preferably in an amount between about 5 and 20 wt %, and more preferably in an amount between about 5 and 15 wt %.

The sweetener should have a relative sweetness value between about 0.4 and 2500, as compared with sucrose, more typically between about 0.4 and 500, preferably between about 0.4 and 200, and more preferably, between about 0.4 and 2.

In certain embodiments, the sweetener comprises xylitol, sorbitol, fructose, invert sugar, palantinose, mannitol, maltitol, palatinit, ammonium glycyrrhizinate, or a combination of any of the foregoing materials.

The nonnutritive sweetener is typically present in an amount between about 50 and 90 wt %, preferably in an amount between about 70 and 90 wt %, and more preferably in an amount between about 80 and 90 wt %.

The lozenge preferably is a buffered formulation in order to aid in buccal absorption of reduced folate. A preferred formulation is at a pH of about 6-11, and preferably at a pH of about 7-9. P referred buffered formulations will include sodium carbonate, sodium bicarbonate, sodium phosphate, calcium carbonate, magnesium hydroxide, potassium hydroxide, magnesium carbonate, aluminum hydroxide, and other substances known to those skilled in the art, as well as combinations of the aforementioned substances. In a most preferred formulation, the lozenge will contain sodium carbonate and bicarbonate as buffering agents.

The buffering agent(s) should be present in an amount sufficient to adjust the pH of the lozenge to between 6 and 11, typically, between about 0.1 and 25% by weight (wt %), preferably in an amount between about 0.1 and 10 wt %, and more preferably in an amount between about 0.1 and 5 wt %.

In addition, the lozenge may contain a flavorant, for example, a candy taste, such as chocolate, orange, vanilla, and the like; essential oils such as peppermint, spearmint and the like; or other flavor, such as aniseed, eucalyptus, 1-menthol, carvone, anethole and the like, to mask the taste of nicotine. See Hall et al. Food Technol. 14:488 (1960); 15:20 (1961); 19:151 (1965); 24:25 (1970); 26:35 (1972); 27:64 (1973); 27:56 (1973); 28:76 (1974); 29:70 (1974) 31:65 (1977); 32:60 (1978); and 33:65 (1979), each of which is incorporated herein by reference.

Magnesium stearate and/or hydrogenated vegetable oil may also be added to the formulation as lubricants. Typically, the lubricant will be present in an amount between about 0.1 and 25 wt %, preferably in an amount between about 0.1 and 10 wt %, and more preferably in an amount between about 0.1 and 5 wt %.

The lozenges described herein may also contain a variety of other additives. For example, pharmacologically active ingredients such as sodium monofluorophosphate, sodium fluoride, dextranase, mutanase, hinokitiol, allantoin, aminocaproic acid, tranexamic acid, azulene, vitamin E derivatives, sodium chloride and the like can be added at need.

In some embodiments, the lozenge may be colored with conventional, pharmaceutically acceptable food coloring agents. Other additives that may be incorporated within the lozenges described herein include, but are not limited to, preservatives, antimicrobial agents, and antioxidants.

The method of manufacture of these lozenges may comprise a direct compression of a mixture comprising a reduced folate, an absorbent excipient (e.g., mannitol or β-cyclodextrin) and a sweetener.

Gum

In some embodiments, pharmaceutical reduced folate formulations are provided as gum comprising a reduced folate.

In certain embodiments, the gum is intended to release an effective dose of reduced folate over from about 5 minutes to about 60 minutes or from about 5 minutes to about 30 minutes of chewing.

In certain embodiments, the gum comprises the reduced folate dispersed in a gum base.

In certain embodiments, the gum base comprises guar gum.

In certain embodiments, the gum comprises from about 1 mg to about 100 mg of a reduced folate and about 1.5-2.5 g of guar gum, or a different gum, and about 300-500 mg of a physiologically-acceptable metal salt (e.g., magnesium salt).

In some of these embodiments, the gum further comprises an ion-exchange resin, and the reduced folate is bound to the ion-exchange resin.

Liquid Formulations

In some embodiments, pharmaceutical reduced folate formulations are provided as liquid formulations comprising the reduced folate particles described herein and at least one dispersing agent or suspending agent for oral administration to a subject. The reduced folate formulation may be a powder and/or granules for suspension, and upon admixture with water, a substantially uniform suspension is obtained. As described herein, the aqueous dispersion can comprise amorphous and non-amorphous reduced folate particles of consisting of multiple effective particle sizes such that reduced folate particles having a smaller effective particle size are absorbed more quickly and reduced folate particles having a larger effective particle size are absorbed more slowly. In certain embodiments the aqueous dispersion or suspension is an immediate release formulation. In another embodiment, an aqueous dispersion comprising amorphous reduced folate particles is formulated such that about 50% of the reduced folate particles are absorbed within about 3 hours after administration and about 90% of the reduced folate particles are absorbed within about 10 hours after administration. In other embodiments, addition of a complexing agent to the aqueous dispersion results in a larger span of reduced folate containing particles to extend the drug absorption phase such that 50-80% of the particles are absorbed in the first 3 hours and about 90% are absorbed by about 10 hours.

A suspension is “substantially uniform” when it is mostly homogenous, that is, when the suspension is composed of approximately the same concentration of reduced folate at any point throughout the suspension. Preferred embodiments are those that provide concentrations essentially the same (within 15%) when measured at various points in a reduced folate aqueous oral formulation after shaking. Especially preferred are aqueous suspensions and dispersions, which maintain homogeneity (up to 15% variation) when measured 2 hours after shaking. The homogeneity should be determined by a sampling method consistent with regard to determining homogeneity of the entire composition. In one embodiment, an aqueous suspension can be re-suspended into a homogenous suspension by physical agitation lasting less than 1 minute. In another embodiment, an aqueous suspension can be re-suspended into a homogenous suspension by physical agitation lasting less than 45 seconds. In yet another embodiment, an aqueous suspension can be re-suspended into a homogenous suspension by physical agitation lasting less than 30 seconds. In still another embodiment, no agitation is necessary to maintain a homogeneous aqueous dispersion.

In some embodiments, the reduced folate powders for aqueous dispersion described herein comprise stable reduced folate particles having an effective particle size by weight of less than 500 nm formulated with reduced folate particles having an effective particle size by weight of greater than 500 nm. In such embodiments, the formulations have a particle size distribution wherein about 10% to about 100% of the reduced folate particles by weight are between about 75 nm and about 500 nm, about 0% to about 90% of the reduced folate particles by weight are between about 150 nm and about 400 nm, and about 0% to about 30% of the reduced folate particles by weight are greater than about 600 nm. The reduced folate particles describe herein can be amorphous, semi-amorphous, crystalline, semi-crystalline, or mixture thereof.

In one embodiment, the aqueous suspensions or dispersions described herein comprise reduced folate particles or reduced folate complex at a concentration of about 20 mg/ml to about 150 mg/ml of suspension. In another embodiment, the aqueous oral dispersions described herein comprise reduced folate particles or reduced folate complex particles at a concentration of about 25 mg/ml to about 75 mg/ml of solution. In yet another embodiment, the aqueous oral dispersions described herein comprise reduced folate particles or reduced folate complex at a concentration of about 50 mg/ml of suspension. The aqueous dispersions described herein are especially beneficial for the administration of reduced folate to infants (less than 2 years old), children under 10 years of age and any patient group that is unable to swallow or ingest solid oral dosage forms.

Liquid reduced folate formulation dosage forms for oral administration can be aqueous suspensions selected from the group including, but not limited to, pharmaceutically acceptable aqueous oral dispersions, emulsions, solutions, and syrups. See, e.g., Singh et al., Encyclopedia of Pharmaceutical Technology, 2nd Ed., pp. 754-757 (2002). In addition to reduced folate particles, the liquid dosage forms may comprise additives, such as: (a) disintegrating agents; (b) dispersing agents; (c) wetting agents; (d) at least one preservative, (e) viscosity enhancing agents, (f) at least one sweetening agent, (g) at least one flavoring agent, (h) a complexing agent. and (i) an ionic dispersion modulator. In some embodiments, the aqueous dispersions can further comprise a crystalline inhibitor.

Examples of disintegrating agents for use in the aqueous suspensions and dispersions include, but are not limited to, a starch, e.g., a natural starch such as corn starch or potato starch, a pregelatinized starch such as National 1551 or Amijel®, or sodium starch glycolate such as Promogel® or Explotab®; a cellulose such as a wood product, microcrystalline cellulose, e.g., Avicel®, Avicel® PH101, Avicel® PH102, Avicel® PH105, Elcema® P100, Emcocel®, Vivacel®, Ming Tia®, and Solka-Floc®, methylcellulose, croscarmellose, or a cross-linked cellulose, such as cross-linked sodium carboxymethylcellulose (Ac-Di-Sol®), cross-linked carboxymethylcellulose, or cross-linked croscarmellose; a cross-linked starch such as sodium starch glycolate; a cross-linked polymer such as crosspovidone; a cross-linked polyvinylpyrrolidone; alginate such as alginic acid or a salt of alginic acid such as sodium alginate; a clay such as Veegum® HV (magnesium aluminum silicate); a gum such as agar, guar, locust bean, Karaya, pectin, or tragacanth; sodium starch glycolate; bentonite; a natural sponge; a surfactant; a resin such as a cation-exchange resin; citrus pulp; sodium lauryl sulfate; sodium lauryl sulfate in combination starch; and the like.

In some embodiments, the dispersing agents suitable for the aqueous suspensions and dispersions described herein are known in the art and include, for example, hydrophilic polymers, electrolytes, Tween® 60 or 80, PEG, polyvinylpyrrolidone (PVP; commercially known as Plasdone®), and the carbohydrate-based dispersing agents such as, for example, hydroxypropylcellulose and hydroxypropylcellulose ethers (e.g., HPC, HPC-SL, and HPC-L), hydroxypropylmethylcellulose and hydroxypropylmethylcellulose ethers (e.g. HPMC K100, HPMC K4M, HPMC K15M, and HPMC K100M), carboxymethylcellulose sodium, methylcellulose, hydroxyethyl cellulose, hydroxypropylmethylcellulose phthalate, hydroxypropylmethylcellulose acetate stearate, noncrystalline cellulose, magnesium aluminum silicate, triethanolamine, polyvinyl alcohol (PVA), polyvinylpyrrolidone/vinyl acetate copolymer (Plasdone®, e.g., S-630), 4-(1,1,3,3-tetramethylbutyl)-phenol polymer with ethylene oxide and formaldehyde (also known as tyloxapol), poloxamers (e.g., Pluronics F68®, F88®, and F108®, which are block copolymers of ethylene oxide and propylene oxide); and poloxamines (e.g., Tetronic 908®, also known as Poloxamine 908®, which is a tetrafunctional block copolymer derived from sequential addition of propylene oxide and ethylene oxide to ethylenediamine (BASF Corporation, Parsippany, N.J.)). In other embodiments, the dispersing agent is selected from a group not comprising one of the following agents: hydrophilic polymers; electrolytes; Tween® 60 or 80; PEG; polyvinylpyrrolidone (PVP); hydroxypropylcellulose and hydroxypropyl cellulose ethers (e.g., HPC, HPC-SL, and HPC-L); hydroxypropyl methylcellulose and hydroxypropyl methylcellulose ethers (e.g. HPMC K100, HPMC K4M, HPMC K15M, HPMC K100M, and Pharmacoat® USP 2910 (Shin-Etsu)); carboxymethylcellulose sodium; methylcellulose; hydroxyethyl cellulose; hydroxypropylmethyl-cellulose phthalate; hydroxypropylmethyl-cellulose acetate stearate; non-crystalline cellulose; magnesium aluminum silicate; triethanolamine; polyvinyl alcohol (PVA); 4-(1,1,3,3-tetramethylbutyl)-phenol polymer with ethylene oxide and formaldehyde; poloxamers (e.g., Pluronics F68®, F88®, and F108®, which are block copolymers of ethylene oxide and propylene oxide); or poloxamines (e.g., Tetronic 908®, also known as Poloxamine 908®).

Wetting agents (including surfactants) suitable for the aqueous suspensions and dispersions described herein are known in the art and include, but are not limited to, acetyl alcohol, glycerol monostearate, polyoxyethylene sorbitan fatty acid esters (e.g., the commercially available Tweens® such as e.g., Tween 20® and Tween 80® (ICI Specialty Chemicals)), and polyethylene glycols (e.g., Carbowaxs 3350® and 1450®, and Carpool 934® (Union Carbide)), oleic acid, glyceryl monostearate, sorbitan monooleate, sorbitan monolaurate, triethanolamine oleate, polyoxyethylene sorbitan monooleate, polyoxyethylene sorbitan monolaurate, sodium oleate, sodium lauryl sulfate, sodium docusate, triacetin, vitamin E TPGS, sodium taurocholate, simethicone, phosphotidylcholine and the like.

Suitable preservatives for the aqueous suspensions or dispersions described herein include, for example, potassium sorbate, parabens (e.g., methylparaben and propylparaben) and their salts, benzoic acid and its salts, other esters of parahydroxybenzoic acid such as butylparaben, alcohols such as ethyl alcohol or benzyl alcohol, phenolic compounds such as phenol, or quaternary compounds such as benzalkonium chloride. Preservatives, as used herein, are incorporated into the dosage form at a concentration sufficient to inhibit microbial growth. In one embodiment, the aqueous liquid dispersion can comprise methylparaben and propylparaben in a concentration ranging from about 0.01% to about 0.3% methylparaben by weight to the weight of the aqueous dispersion and 0.005% to 0.03% propylparaben by weight to the total aqueous dispersion weight. In yet another embodiment, the aqueous liquid dispersion can comprise methylparaben 0.05 to about 0.1 weight % and propylparaben from 0.01-.02 weight % of the aqueous dispersion.

Suitable viscosity enhancing agents for the aqueous suspensions or dispersions described herein include, but are not limited to, methyl cellulose, xanthan gum, carboxymethylcellulose, hydroxypropyl cellulose, hydroxypropylmethyl cellulose, Plasdone® S-630, carbomer, polyvinyl alcohol, alginates, acacia, chitosans and combinations thereof. The concentration of the viscosity enhancing agent will depend upon the agent selected and the viscosity desired.

Examples of natural and artificial sweetening agents suitable for the aqueous suspensions or dispersions described herein include, for example, acacia syrup, acesulfame K, alitame, anise, apple, aspartame, banana, Bavarian cream, berry, black currant, butterscotch, calcium citrate, camphor, caramel, cherry, cherry cream, chocolate, cinnamon, bubble gum, citrus, citrus punch, citrus cream, cotton candy, cocoa, cola, cool cherry, cool citrus, cyclamate, cylamate, dextrose, eucalyptus, eugenol, fructose, fruit punch, ginger, glycyrrhetinate, glycyrrhiza (licorice) syrup, grape, grapefruit, honey, isomalt, lemon, lime, lemon cream, monoammonium glyrrhizinate (MagnaSweet®), maltol, mannitol, maple, marshmallow, menthol, mint cream, mixed berry, neohesperidine DC, neotame, orange, pear, peach, peppermint, peppermint cream, Prosweet® Powder, raspberry, root beer, rum, saccharin, safrole, sorbitol, spearmint, spearmint cream, strawberry, strawberry cream, stevia, sucralose, sucrose, sodium saccharin, saccharin, aspartame, acesulfame potassium, mannitol, talin, sucralose, sorbitol, Swiss cream, tagatose, tangerine, thaumatin, tutti fruitti, vanilla, walnut, watermelon, wild cherry, wintergreen, xylitol, or any combination of these flavoring ingredients, e.g., anise-menthol, cherry-anise, cinnamon-orange, cherry-cinnamon, chocolate-mint, honey-lemon, lemon-lime, lemon-mint, menthol-eucalyptus, orange-cream, vanilla-mint, and mixtures thereof. In one embodiment, the aqueous liquid dispersion can comprise a sweetening agent or flavoring agent in a concentration ranging from about 0.0001% to about 10.0% the weight of the aqueous dispersion. In another embodiment, the aqueous liquid dispersion can comprise a sweetening agent or flavoring agent in a concentration ranging from about 0.0005% to about 5.0 wt % of the aqueous dispersion. In yet another embodiment, the aqueous liquid dispersion can comprise a sweetening agent or flavoring agent in a concentration ranging from about 0.0001% to 0.1 wt %, from about 0.001% to about 0.01 weight %, or from 0.0005% to 0.004% of the aqueous dispersion.

In addition to the additives listed above, the liquid reduced folate formulations can also comprise inert diluents commonly used in the art, such as water or other solvents, solubilizing agents, and emulsifiers.

### Emulsions

In some embodiments, the pharmaceutical reduced folate formulations described herein can be self-emulsifying drug delivery systems (SEDDS). Emulsions are dispersions of one immiscible phase in another, usually in the form of droplets. Generally, emulsions are created by vigorous mechanical dispersion. SEDDS, as opposed to emulsions or microemulsions, spontaneously form emulsions when added to an excess of water without any external mechanical dispersion or agitation. An advantage of SEDDS is that only gentle mixing is required to distribute the droplets throughout the solution. Additionally, water or the aqueous phase can be added just prior to administration, which ensures stability of an unstable or hydrophobic active ingredient. Thus, the SEDDS provides an effective delivery system for oral and parenteral delivery of hydrophobic active ingredients. SEDDS may provide improvements in the bioavailability of hydrophobic active ingredients. Methods of producing self-emulsifying dosage forms are known in the art include, but are not limited to, for example, U.S. Pat. Nos. 5,858,401, 6,667,048, and 6,960,563, each of which is specifically incorporated by reference.

Exemplary emulsifiers are ethyl alcohol, isopropyl alcohol, ethyl carbonate, ethyl acetate, benzyl alcohol, benzyl benzoate, propylene glycol, 1,3-butyleneglycol, dimethylformamide, sodium lauryl sulfate, sodium doccusate, cholesterol, cholesterol esters, taurocholic acid, phosphotidylcholine, oils, such as cottonseed oil, groundnut oil, corn germ oil, olive oil, castor oil, and sesame oil, glycerol, tetrahydrofurfuryl alcohol, polyethylene glycols, fatty acid esters of sorbitan, or mixtures of these substances, and the like.

### Transdermal Formulations

Transdermal formulations described herein may be administered using a variety of devices which have been described in the art. For example, such devices include, but are not limited to, U.S. Pat. Nos. 3,598,122, 3,598,123, 3,710,795, 3,731,683, 3,742,951, 3,814,097, 3,921,636, 3,972,995, 3,993,072, 3,993,073, 3,996,934, 4,031,894, 4,060,084, 4,069,307, 4,077,407, 4,201,211, 4,230,105, 4,292,299, 4,292,303, 5,336,168, 5,665,378, 5,837,280, 5,869,090, 6,923,983, 6,929,801 and 6,946,144, each of which is specifically incorporated by reference in its entirety. In some embodiments, the transdermal delivery device used with the reduced folate formulations described herein can comprise a power source, radio frequency, or a brief electrical current to micro-electrodes in the skin creating “channels” or “pores” in the stratum corneum to facilitate the delivery of the reduced folate formulation, such methods are known in the art and are described in, for example U.S. Pat. Nos. 6,611,706, 6,708,060, and 6,711,435, each of which is specifically incorporated by reference in its entirety. In other embodiments, the transdermal delivery device can comprise a means for porating the stratum corneum, e.g., micro-lancing, application of sonic energy, or hydraulic puncturing, to facilitate the delivery of the reduced folate formulation, such methods are known in the art and are described in, for example, U.S. Pat. Nos. 6,142,939 and 6,527,716, each of which is specifically incorporated by reference in its entirety. The pores described by the methods herein are typically about 20-50 microns in depth and to not extend into areas of innervation or vascularization.

The transdermal dosage forms described herein may incorporate certain pharmaceutically acceptable excipients which are conventional in the art. In general, the transdermal formulations described herein comprise at least three components: (1) a reduced folate or reduced folate complex formulation; (2) a penetration enhancer; and (3) an aqueous adjuvant. In addition, transdermal formulations can include additional components such as, but not limited to, gelling agents, creams and ointment bases, and the like. In some embodiments, the transdermal formulation can further comprise a woven or non-woven backing material to enhance drug absorption and prevent the removal of the transdermal formulation from the skin. In other embodiments, the transdermal formulations described herein can maintain a saturated or supersaturated state to promote diffusion into the skin.

### Injectable Formulations

Reduced folate formulations suitable for intramuscular, subcutaneous, or intravenous injection may comprise physiologically acceptable sterile aqueous or non-aqueous solutions, dispersions, suspensions or emulsions, and sterile powders for reconstitution into sterile injectable solutions or dispersions. Examples of suitable aqueous and non-aqueous carriers, diluents, solvents, or vehicles including water, ethanol, polyols (propylene glycol, polyethylene-glycol, glycerol, cremophor and the like), suitable mixtures thereof, vegetable oils (such as olive oil) and injectable organic esters such as ethyl oleate. Additionally, reduced folate can be dissolved at concentrations of >1 mg/ml using water soluble beta cyclodextrins (e.g. beta-sulfobutyl-cyclodextrin and 2-hydroxypropylbetacyclodextrin. Proper fluidity can be maintained, for example, by the use of a coating such as lecithin, by the maintenance of the required particle size in the case of dispersions, and by the use of surfactants. Reduced folate formulations suitable for subcutaneous injection may also contain additives such as preserving, wetting, emulsifying, and dispensing agents. Prevention of the growth of microorganisms can be ensured by various antibacterial and antifungal agents, such as parabens, benzoic acid, benzyl alcohol, chlorobutanol, phenol, sorbic acid, and the like. It may also be desirable to include isotonic agents, such as sugars, sodium chloride, and the like. Prolonged drug absorption of the injectable pharmaceutical form can be brought about by the use of agents delaying absorption, such as aluminum monostearate and gelatin. Reduced folate suspension formulations designed for extended release via subcutaneous or intramuscular injection can avoid first pass metabolism and lower dosages of reduced folate will be necessary to maintain plasma levels of about 50 ng/ml. In such formulations, the particle size of the reduced folate particles and the range of the particle sizes of the reduced folate particles can be used to control the release of the drug by controlling the rate of dissolution in fat or muscle.

Parenteral solution formulations of reduced folates (e.g., levoleucovorin) may be further diluted to concentrations of 0.5 mg/mL in 0.9% Sodium Chloride Injection, USP or 5% Dextrose Injection, USP. The diluted solution using 0.9% Sodium Chloride Injection, USP or 5% Dextrose Injection, USP may be held at room temperature for not more than 4 hours. In embodiments wherein the reduced folate is levoleucovorin calcium, no more than 16 mL (160 mg of Levoleucovorin) should be injected intravenously per minute, because of the Ca++ content of the Levoleucovorin solution. Levoleucovorin injection is commercially available in a single dose vial, e.g., at 175 mg/17.5 ml (10 mg/ml), wherein the active drug is present as levoleucovorin calcium. The vial further includes sodium chloride 8.3 mg/ml and sodium hydroxide for pH adjustment to pH 8.1 (pH range from about 6.5 to about 8.5), and does not contain preservatives.

### Sterile Reduced Folate Formulations

Some of the reduced folate formulations described herein can be sterile filtered. This property obviates the need for heat sterilization, which can harm or degrade reduced folate, as well as result effective particle size growth.

Sterile filtration can be difficult because of the required small particle size of the composition. However, this method is suitable and commonly used for dispersions comprising nanoparticles. Filtration is an effective method for sterilizing homogeneous solutions when the membrane filter pore size is less than or equal to about 0.2 microns (200 nm) because a 0.2 micron filter is sufficient to remove essentially all bacteria. Sterile filtration is normally not used to sterilize conventional suspensions of micron-sized reduced folate because the reduced folate particles are too large to pass through the membrane pores.

Because some of the reduced folate-complex formulations described herein can be sterilized via autoclaving, and because the formulations can have a very small reduced folate effective average particle size, some sterilized reduced folate formulations are suitable for parenteral administration. Additionally, a sterile reduced folate formulation is particularly useful in treating immunocompromised patients, infants or juvenile patients, patients with head trauma and the elderly.

Combination Therapies

The compositions and methods described herein may also be used in conjunction with other well-known therapeutic reagents that are selected for their particular usefulness against the condition that is being treated. In general, the compositions described herein and, in embodiments where combinational therapy is employed, other agents do not have to be administered in the same pharmaceutical composition, and may, because of different physical and chemical characteristics, have to be administered by different routes. The determination of the mode of administration and the advisability of administration, where possible, in the same pharmaceutical composition, is well within the knowledge of the skilled clinician. The initial administration can be made according to established protocols known in the art, and then, based upon the observed effects, the dosage, modes of administration and times of administration can be modified by the skilled clinician.

The particular choice of compounds used will depend upon the diagnosis of the attending physicians and their judgment of the condition of the patient and the appropriate treatment protocol. The compounds may be administered concurrently (e.g., simultaneously, essentially simultaneously or within the same treatment protocol) or sequentially, depending upon the nature of the proliferative disease, the condition of the patient, and the actual choice of compounds used. The determination of the order of administration, and the number of repetitions of administration of each therapeutic agent during a treatment protocol, is well within the knowledge of the skilled physician after evaluation of the disease being treated and the condition of the patient.

It is understood that the dosage regimen to treat, prevent, or ameliorate the condition(s) for which relief is sought, can be modified in accordance with a variety of factors. These factors include the disorder from which the subject suffers, as well as the age, weight, sex, diet, and medical condition of the subject. Thus, the dosage regimen actually employed can vary widely and therefore can deviate from the dosage regimens set forth herein.

The pharmaceutical agents which make up the combination therapy disclosed herein may be a combined dosage form or in separate dosage forms intended for substantially simultaneous administration. The pharmaceutical agents that make up the combination therapy may also be administered sequentially, with either therapeutic compound being administered by a regimen calling for two-step administration. The two-step administration regimen may call for sequential administration of the active agents or spaced-apart administration of the separate active agents. The time period between the multiple administration steps may range from, a few minutes to several hours, depending upon the properties of each pharmaceutical agent, such as potency, solubility, bioavailability, plasma half-life and kinetic profile of the pharmaceutical agent. Circadian variation of the target molecule concentration may also determine the optimal dose interval.

In certain embodiments, the combination therapy comprising administering the reduced folate in a combination with a nutraceutical. Nutraceuticals include, e.g., products may include: minerals, herbs or other botanicals, amino acids, and substances such as enzymes, organ tissues, glandulars, and metabolites.

In certain embodiments, the combination therapy comprising administering the reduced folate in a combination with another vitamin. Another vitamin may, e.g., be selected form the group consisting of vitamin A, vitamin B1, vitamin B2, vitamin B3, vitamin B5, vitamin B6, vitamin B7, vitamin B8 (inositol), vitamin B12, vitamin C, vitamin E, vitamin D, and combination thereof.

In certain embodiments, the combination therapy comprising administering the reduced folate in a combination with iron.

Instruments for Cognitive Function Assessments and Instruments

Measuring cognitive function, including language, is central for diagnosing disorders associated with brain function, including neurological, neurodegenerative and neurodevelopmental disorders. These instruments are designed to diagnose cognitive function as normal or abnormal and categorize cognitive impairments or strengths into large categories. As such instruments are used for diagnosis, they are not designed to indicate the extent of impairments or strengths beyond a certain point as such information would not be of use in labeling an individual with a diagnosis.

Typical cognitive instruments, including those that measure language, are designed to measure cognitive function at one point in time and provide a label for individual's abilities at that particular point in time. Instruments do not produce measurement in a way to allow them to be compared to another point in time. Although some individuals do try to use the measurements from standard cognitive measurement instruments to examine change, this measure of change may not be valid in many cases because the measurement scales have an upper (ceiling) or lower (floor) limit of their measurement. Thus, if the individual's skills is below the floor (as is commonly the case) or above the ceiling, the measurements will not be accurate and change in skills over time may be zero because the measurements at the two time points are both below or above the measurement capability of the instrument used.

The Clinical Evaluation of Language Fundamentals (“CELF”) is a standardized, well-validated instrument that assesses skills that are abnormal in ASD and has been used in studies focusing on verbal communication in ASD. The PLS-5 is a standardized, well-validated instrument that measures subtle changes in verbal communication, particularly in preverbal children.

The Ohio Autism Clinical Impression Scale (“OACIS”) is an observer-rated scale sensitive to clinically meaningful changes in ASD symptoms. It was first developed as the Ohio State University Autism Rating Scale and has been shown to have good inter-rater and cross-cultural reliability and has been successfully used in several ASD clinical trials. Severity of each symptom was rated by the first author at baseline and at the final assessment by observing the entire assessment of verbal communication. In validation studies a 0.5-point change was considered clinically meaningful.

The Vineland Adaptive Behavior Scale 2nd Edition (“VABS”) is a reliable and valid measure of the ability to perform age-appropriate everyday skills though a 20-30 min structured interview with a caretaker. Standard scores from the communication, daily living, social and motor skills, and adaptive behavioral composite were analyzed. Standard scores have a mean of 100, standard deviation of 15 and range 20-160. Intervention studies in ASD have demonstrated a change of 6 points to be clinically meaningful.

The Aberrant Behavior Checklist (“ABC”) is a 58-item questionnaire that measures disruptive behaviors, including Irritability (15 items, range 0-45); Social Withdrawal (16 items, range 0-48); Stereotypy (7 items, range 0-21); Hyperactivity (16 items, range 0-48) and Inappropriate Speech (4 items, range 0-12). Each item is rated 0 to 3 with higher scores indicating greater severity. Multiple ASD clinical trials have used it and it has convergent and divergent validity. Interventional ASD studies suggest a 12-point decrease in the total score is clinically meaningful.

The Behavioral Assessment System for Children 2nd Edition (“BASC”) ranges from 185 to 306 items and is validated in ASD. Each item is rated 0 to 3 with higher scores indicating greater severity. Standardized T-Scores (mean 50, standard deviation 10) range 20-120 for externalizing, internalizing and behavioral symptoms and 10-90 for adaptive skills.

The Social Responsiveness Scale (“SRS”) is a 65-item questionnaire that measures social skills across five domains: Social Awareness (8 items, meaningful change 7.1), Social Cognition (12 items, meaningful change 5.8), Social Communication (22 items, meaningful change 4.2), Social Motivation (11 items, meaningful change 5.7), Autistic Mannerisms (12 items, meaningful change 5.5) and total (65 items). Each item is rated 0 to 3 with higher scores indicating greater severity. Standardized T-scores (mean 50, standard deviation 10) range 30-90.

The Autism Impact Measure (“AIM”) is a 45-item parent-reported measure of the frequency and impact of core ASD symptoms during the past 2 weeks using two 5-point scales of increasing severity ranging from 1 to 5.51 The Frequency and Impact scores range 45-225.

The Autism Symptoms Questionnaire (“ASQ) is a 34-item checklist (The Center for Autism and Related Disorders) that assesses social interaction (12 items, range 0-4), stereotyped behavior (7 items, range 0-4), communication symptoms (15 items, range 0-5) and total symptoms (34 items, range 0-13). Intervention ASD studies suggest a 1.1 point change as clinically meaningful.

Cognitive instruments have a “floor” or “ceiling” below which and above which they do not provide measurements. This might be fine for diagnostic purposes, but it results in significant problems when the change in cognition, including language, needs to be followed over time as is needed in following the course of a disorder or measuring the effect of a treatment. For example, it is not unusual for individuals to be impaired to the point that they are measured to have abilities below the floor of a cognitive test. In such cases it is very difficult to determine if the individual improves over time since even with some improvement they will still be judged to below the floor of the test and will thus appear to have no change in ability on the test conducted. Thus, an individual with a significant impairment or gift will test outside the testable range of the instrument. In other words, the instrument has limited dynamic range to measure any change in the abilities of such an individual. In such cases there is no protocol or method for determining their impairment beyond being outside of the range of the instrument.

One of the reasons that individual scores outside of the range is because instruments are chosen to measure abilities based on the age of the individual. Thus, an individual who is functioning well below their chronological age will fall below the testing limit of the instrument and the instrument will not be useful to measure the individual's real abilities.

It has become well known that individuals with neurodevelopmental disorders can attain abilities to the point where, for all intents and purposes, they have lost their diagnosis, yet tools are not made to follow the trajectory of improvement or worsening over time. This is also important in neurodegenerative disorders where it is becoming clear that the rate of loss of skills can help define whether an individual is going to develop pathological disease like Alzheimer's disease or go on to have a more mild course consistent with normal aging like minimal cognitive impairment. Currently, instruments are limited because they try to use a measurement at one point in time to make an assessment. Additionally, clinical trials on interventions have resulting in uninterpretable results because the instrument used to measure change in ability was not designed to do so and failed to provide the fidelity required to make an objective assessment.

In order to circumvent these problems, the inventors developed a method that uses several available instruments together to determine the most ability appropriate instrument for an individual and then use that instrument to determine the individuals true abilities. Once the instrument is found that can measure the individual's abilities, the method uses that instrument in future assessments of cognitive ability to ensure that changes in abilities can be measured appropriately. In some embodiments, from two to five instruments are used to determine individual's verbal communication and obtain a verbal communication score that can be used during the baseline assessment and subsequent assessments.

In some embodiments, the method comprises testing the verbal communication of a subject (e.g., a child) with a first age-appropriate instrument to obtain a first score, and, if the first score is at a floor of the first age-appropriate instrument, testing the verbal communications of the subject with the next lower ability instrument to obtain a second score, and, if the second score is at a floor of the next lower ability instrument, repeating the process until a final score above a floor of the final ability instrument is obtained. The score from the final instrument is then used during the baseline assessment and subsequent assessments. In certain embodiments, the first age-appropriate instrument, the second age-appropriate instrument and subsequent age-appropriate instruments are selected from the group consisting of CELF-preschool-2, CELF-4, Preschool Language Scale-5 (PLS-5), and combinations thereof.

The method allows, e.g., measuring the change in cognitive or language development in individuals with normal and atypical development. This includes evaluating infants, children, adolescents and adults with developmental delays, learning disabilities, attention deficit disorders, autism spectrum disorders or other disorders in which cognition and/or language is affected. This also includes individuals with normal development to follow their development along optimally.

The method can be used in neurodegenerative disorders in infants, children, adolescents, adults and elderly to follow the loss of skills in cognitive, language as well as other functions of daily living.

As demonstrated in the examples below, this method is optimally suited for measuring changes in cognition and/or language as a result of an intervention. The intervention could be experimental (as in the example below) or can be a standard intervention.

For example, the method can, e.g., be used to determine if an individual is responding to standard intervention. In this manner this method can be used in personalized medicine or precision medicine to determine the optimal therapy for an individual.

In one aspect, the novelty of the method is related to improving in the ability to detect change in cognitive abilities as a result of nature or as a result of a specific or non-specific interventions. Unlike, the tools are developed to measure ability at one point in time without regards to the ability of such measurements to be compared over time and are designed to make a diagnosis which is then believe to be static, the methods and instruments of the invention allow the natural state of a neurodevelopmental, neurodegenerative or neurological disease to be monitoring accurately and allows the accurate measurement of the effect of an intervention to be monitored.

### DETAILED DESCRIPTION OF THE PREFERRED EMBODIMENTS

The following examples illustrate various aspects of the present invention. They are not to be construed to limit the claims in any manner whatsoever. It will be readily apparent to one of ordinary skill in the relevant arts that other suitable modifications and adaptations to the methods and applications described herein are suitable and may be made without departing from the scope of the invention or any embodiment thereof. While the invention has been described in connection with certain embodiments, it is not intended to limit the invention to the particular forms set forth, but on the contrary, it is intended to cover such alternatives, modifications and equivalents as may be included within the spirit and scope of the invention as defined by the following claims.

### Example 1

In a controlled open-label study, we found that children with ASD who were positive for at least one FRAA experienced significant improvements in verbal communication, receptive and expressive language, attention, and stereotypical behavior with high-dose (2 mg kg⁻¹ per day in two divided doses; maximum 50 mg per day) folinic acid treatment with very few adverse effects reported.

### Example 2

To determine whether high-dose folinic acid can improve core and associated ASD symptoms, we conducted a single-site randomized double-blind placebo-controlled clinical trial. It was hypothesized that high-dose folinic acid would alleviate ASD symptoms, particularly in children with folate-related metabolic abnormalities. In addition, we sought to determine if biomarkers of disruptions in folate metabolism, such as the FRAA, could predict which children would respond to folinic acid treatment, so that invasive diagnostic procedures such as a lumbar puncture might be avoided.

We sought to determine whether high-dose folinic acid improves verbal communication in children with non-syndromic autism spectrum disorder (ASD) and language impairment in a double-blind placebo control setting. Forty-eight children (mean age 7 years 4 months; 82% male) with ASD and language impairment were randomized to receive 12 weeks of high-dose folinic acid (2 mg kg⁻¹ per day, maximum 50 mg per day; n=23) or placebo (n=25). Children were subtyped by glutathione and folate receptor-α autoantibody (FRAA) status. Improvement in verbal communication, as measured by an ability-appropriate standardized instrument, was significantly greater in participants receiving folinic acid as compared with those receiving placebo, resulting in an effect of 5.7 (1.0,10.4) standardized points with a medium-to-large effect size (Cohen's d=0.70). FRAA status was predictive of response to treatment. For FRAA-positive participants, improvement in verbal communication was significantly greater in those receiving folinic acid as compared with those receiving placebo, resulting in an effect of 7.3 (1.4,13.2) standardized points with a large effect size (Cohen's d=0.91), indicating that folinic acid treatment may be more efficacious in children with ASD who are FRAA positive. Improvements in subscales of the Vineland Adaptive Behavior Scale, the Aberrant Behavior Checklist, the Autism Symptom Questionnaire and the Behavioral Assessment System for Children were significantly greater in the folinic acid group as compared with the placebo group. There was no significant difference in adverse effects between treatment groups. Thus, in this small trial of children with non-syndromic ASD and language impairment, treatment with high-dose folinic acid for 12 weeks resulted in improvement in verbal communication as compared with placebo, particularly in those participants who were positive for FRAAs.

The study was approved by the Institutional Review Board at the University of Arkansas for Medical Sciences (Little Rock, Ark., USA). Parents of participants provided written informed consent.

### Study Design

This two-arm double-blind randomized placebo-controlled parallel study with a 1:1 allocation was performed at Arkansas Children's Research Institute (Little Rock, Ark., USA) from 4 Jun. 2012 to 22 Nov. 2013.

Participants who met inclusion and exclusion criteria were screened for language impairment. Preverbal (<25 functional words) children qualified as language impaired. Otherwise, the age-appropriate version of the Clinical Evaluation of Language Fundamentals (CELF) confirmed language impairment. Language impairment was defined as a core standardized score<85 if the preschool version was used or failure on the CELF screener if other versions were used.

Those confirmed to have language impairment were randomized to the folinic acid or placebo group and a fasting blood sample was obtained. Randomization was performed using a random number generator with a block size of four. The research pharmacists had exclusive access to the randomization allocation. After breakfast, the participants returned for language, developmental and behavioral assessments. Following these assessments the family was given the 12 weeks of the intervention and was instructed on its administration. Language, developmental and behavioral assessments were repeated after 12 weeks of treatment.

Parent and teacher questionnaires were requested at baseline and 6 and 12 weeks after starting treatment. Parents were asked to deliver baseline questionnaires to teachers or therapists. After the first visit, questionnaires were mailed to the parents and teachers at least 1 week prior to the target date of completion. Parents were asked to bring the completed teacher and parent 12-week questionnaires to the final assessment. Other questionnaires were returned in a preaddressed postage-paid envelope.

### Intervention

The target dose of the intervention (INN: DL folinic acid calcium salt; USAN: leucovorin calcium) was 2 mg kg⁻¹ per day (maximum 50 mg per day) in two equally divided doses with half of the target dose given during the first 2 weeks. Dye-free, milk-product-free, vegetarian capsules were provided in three strengths (5, 10 and 25 mg) by Lee Silsby Compounding Pharmacy (Cleveland Heights, Ohio, USA). Certificate of analysis was provided for each capsule strength by an independent analytical service (Eagle Analytical Services, Houston, Tex., USA) for each batch of capsules produced. In all cases, potency was at least 99%.

To verify that folinic acid and placebo capsules were indistinguishable by sight and feel, 10 scientists, 10 medical staff and 10 parents of children with ASD not involved in the study were asked to sort eight small plastic numbered bags, each containing two same strength capsules, into two groups (placebo and folinic acid) of four based upon capsule similarity. No one was able to accurately sort these bags (Binomial P=0.04). Parents were instructed that capsules could be opened and the powder added to food or drink if swallowing the medication was difficult for the child. Both the placebo and folinic acid powder were odorless and tasteless. No parent or child commented on the odor or taste of the medication, providing further evidence of the tasteless and odorless nature of the treatment.

Parents were asked about missed doses and returned pill containers were examined for adherence which was calculated by the research pharmacy.

### Inclusion and Exclusion Criteria

Participants were recruited from our research registry (48%), autism clinic (23%), community advertisement and social media (13%), word-of-mouth (10%) and physician referrals (2%). The ASD diagnosis was defined by one of the following: (i) a gold-standard diagnostic instrument such as the Autism Diagnostic Observation Schedule and/or Autism Diagnostic Interview-Revised; (ii) the state of Arkansas diagnostic standard, defined as agreement of a physician, psychologist and speech therapist; and/or (iii) Diagnostic Statistical Manual (DSM) diagnosis by a physician along with standardized validated questionnaires and diagnosis confirmation by the Principal Investigator. Reconfirmation of the diagnosis using the lifetime version of the Autism Diagnostic Interview-Revised by an independent research reliable rater was requested from all participants.

Inclusion criteria included: (i) age 3-14 years of age; (ii) documentation of language impairment; (iii) unchanged complementary, traditional, behavioral and education therapy 8 weeks prior to enrollment; and (iv) intention to maintain ongoing therapies constant throughout the trial.

Exclusion criteria included: (i) antipsychotic medications; (ii) supplementation exceeding the recommended daily allowance; (iii) prematurity; (iv) uncontrolled gastroesophageal re ux; (v) history of liver or kidney disease; (vi) drugs known to affect folate metabolism (see Supplementary Material); (vii) profound sensory deficits; (viii) well-defined genetic syndromes; (ix) genetic mutations known to significantly affect folate-associated pathways; (x) brain malformations or damage found on magnetic resonance imaging; (xi) ongoing therapies that could interfere with the study; (xii) a clinical seizure within the last 6 months; and (xiii) moderate-to-severe irritability or self-abusive behavior on the aberrant behavior checklist.

### Outcome Measures

All primary and secondary outcomes were obtained at baseline and study end. Questionnaires were also requested 6 weeks after starting the intervention. Aside from the research pharmacists, study staff, participants, parents and teachers were blind to treatment allocation.

Primary outcome. Verbal communication was the primary outcome for several reasons. First, verbal communication improved in preliminary folinic acid treatment studies. Second, verbal communication in children with ASD is closely linked to parental quality of life. Third, the development of language and communication skills is associated with favorable outcomes.

It should be acknowledged that communication impairment was considered a core feature of ASD up until the DSM-V, which has now combined communication and social symptoms into a social-communication symptom cluster. In the DSM-V language impairment is recognized as a significant comorbidity interrelated to the ASD diagnosis. Verbal communication was assessed by an ability-appropriate instrument.

Instruments used were the CELF-preschool-2, CELF-4 and the Preschool Language Scale-5 (PLS-5). The standardized summary score of each instrument (mean 100, standard deviation 15) was the primary outcome measure and ranges from 50 to 150 for the PLS-5 and 45 to 155 for the CELF.

The ability-appropriate instrument was selected using a structured algorithm. The goal was to select an instrument with an adequate dynamic range for assessing improvement in verbal communication. The assessment started with the most age-appropriate instrument.

If the child obtained a score at the floor, the next lower ability instrument was then used. This process was repeated until a score above the floor could be obtained. The score from the final instrument was the primary outcome measure at baseline and at trial end. If the child's age exceeded the maximum age of the instrument's standardization, the maximum standardized age was used. At trial end, all instruments used during the baseline assessment were repeated in the same order to simulate the same baseline assessment experience and to minimize a potential confounder of cognitive fatigue.

Studies have shown that early behavioral therapy improves verbal communication by one-standard deviation over 36 weeks. Thus, a clinically meaningful change was defined as a 5-point increase in verbal communication in this 12-week study since the primary outcome has a 15 point standard deviation. Examining the standard error of participants in the current study suggests that a minimal clinically important difference is 2 points.

Secondary outcomes. Secondary outcome measures included the Ohio Autism Clinical Impression Scale (OACIS), Vineland Adaptive Behavior Scale 2nd Edition (VABS) Survey Interview Form and several questionnaires. Parents and teachers were asked to complete the Aberrant Behavior Checklist (ABC), Social Responsiveness Scale (SRS) and Behavioral Assessment System for Children 2nd Edition (BASC). Only parents were asked to complete the Autism Impact Measure (AIM) and Autism Symptoms Questionnaire (ASQ).

Biomarkers. Two folate-related biomarkers were investigated. FRAA titers, both blocking and binding, were analyzed. Plasma free reduced-to-oxidized glutathione redox ratio was determined. Folate-related vitamins and minerals were measured. Serum total folate and vitamin B12 were measured using MP Diagnostics SimulTRAC-SNB Radioassay Kit (Cat#06B264806). Plasma zinc, whole blood copper and red blood cell magnesium were analyzed by Doctor's Data.

### Establishment and Maintenance of Assessment Fidelity

Research staff was trained by a multispecialty team consisting of two licensed psychologists and a speech therapist prior to performing assessments. During the trial a research psychologist supervised research staff and provided feedback and retraining if necessary.

### Adverse Effects Monitoring

Adverse events were monitored every 3 weeks using a modification of the Dosage Record Treatment Emergent Symptom Scale. Adverse events were considered related to the treatment if they started or worsened following the start of the trial. If adverse events were persistent or severe, the parents were offered the option of halving the dose or discontinuing the intervention. The dose could only be reduced once and was never increased if reduced.

### Statistical Analysis

An intention-to-treat analysis was used. Analyses used SAS version 9.3. To account for missing data multiple imputation was conducted. An imputation of 20 was used and sensitivity analysis was used to check for systematic bias.

Mixed-effects regression models were used to estimate the effect and effect size of the treatment. The models included the effect of time and a random intercept to account for each individual's symptom level. The models tested the a priori hypothesis that the change in the outcome measure was greater for the folinic acid group as compared with the placebo group. This interaction was tested specifically using a two-tailed t-test with a P<0.05. Since our previous study²⁴ demonstrated a large effect size, this study was powered with a large effect size (Cohen's d=0.80), which provided a 77% power with 24 participants per group.

Analyses were conducted on subgroups defined by biomarkers of abnormal folate metabolism. FRAAs were dichotomized as positive and negative and the glutathione redox ratio was dichotomized to relatively high (more normal; above the median of 8.30) and low (more abnormal; below the median of 8.30). Mixed-model regressions, similar to the one described above, were conducted on each subgroup separately since the study was not powered to investigate interaction with these biomarkers using the mixed model.

A responder analysis was conducted using backward elimination (P<0.05 to stay in model) logistic regression. Response was defined by a five standardized point increase in verbal communication since this defines a clinically meaningful change. Age, baseline language and baseline overall development (as indexed by the VABS Behavioral Composite Standardized Score) were entered as potential covariates. To investigate whether the biomarkers of abnormal folate metabolism were related to participant response, logistic regressions were conducted with an interaction between treatment group and each biomarker.

Secondary outcome measures were analyzed using the mixed-model regression. Because of the large number of secondary outcomes, correction for multiple comparisons was conducted using the false discovery rate.

The total number of patients reporting each adverse event was compared across treatment groups using a Fisher exact test. Adverse events that were possibly, probably or definitely related to the treatments were analyzed.

### Results

### Participants

One hundred fifty-six participants were prescreened, with 59 found to potentially meet inclusion/exclusion criteria (FIG. 1), of which 11 failed screening, 10 because of no language impairment and 1 because of congenital hearing impairment. Twenty-five participants were randomized to receive placebo and 23 were randomized to receive high-dose folinic acid (age range 3 years 4 months to 13 years 4 months).

Participant characteristics were similar across treatment groups except for multivitamins (Table 1). Baseline outcome measures were not significantly different across treatment groups except for verbal communication in FRAA-negative participants (F(1,14)=4.58, P=0.05; Tables 2A and 2B). All participants evaluated by an independent research reliable rater exceeded the diagnostic threshold for ASD. The mean number of missed doses per week was not significantly different across groups. Adherence was 490% for those who returned the bottles (20/25 placebo; 16/21 folinic acid).

TABLE 1
Demographic and clinical characteristics by treatment group^{a}

| Variable | Folinic acid (n = 23) | | Placebo (n = 25) | |
|---|---|---|---|---|
| Age, mean (s.d.), years months | 7 y 7 m | (3 y 6 m) | 7 y 2 m | (2 y 10 m) |
| Males, N (%) | 18 | (78) | 20 | (80) |
| Number of missed doses | 0.2 | (0.3) | 0.6 | (1.3) |
| Vineland adaptive behavior | 64.79 | (7.25) | 65.84 | (9.20) |
| composite, mean (s.d.) | | | | |
| Total therapy, minutes per | 339.52 | (498.32) | 500.80 | (591.56) |
| week, mean (s.d.) | | | | |
| Total therapy, minutes per | 195 | (0; 2070) | 300 | (0; 2790) |
| week, mean (s.d.) | | | | |
| Speech therapy, minutes per | 99.52 | (63.26) | 220.00 | (377.77) |
| week, mean (s.d.) | | | | |
| Behavioral therapy, minutes per | 133.57 | (409.75) | 214.80 | (569.58) |
| week, mean (s.d.) | | | | |
| Motor therapy, minutes per | 103.57 | (133.31) | 137.60 | (95.98) |
| week, mean (s.d.) | | | | |
| Folate receptor autoantibody | 13 | (57) | 18 | (72) |
| positive, N (%) | | | | |
| Blocking titer (pmol ml⁻¹), | 0.08 | (0.20) | 0.06 | (0.15) |
| mean (s.d.) | | | | |
| Blocking titer (pmol ml⁻¹), | 0.00 | (0.00, 0.86) | 0.00 | (0.00; 0.55) |
| median (min; max) | | | | |
| Blinding titer (pmol ml⁻¹), | 0.39 | (0.74) | 0.61 | (0.73) |
| mean (s.d.) | | | | |
| Blinding titer (pmol ml⁻¹), | 0.00 | (0.00; 2.46) | 0.38 | (0.00; 2.46) |
| median (min; max) | | | | |
| Glutathione redoc radio, mean | 9.21 | (2.40) | 9.09 | (1.72) |
| (s.d.) | | | | |
| Folate (ng ml⁻¹) | 17.15 | (3.41) | 17.79 | (2.42) |
| (normal 5-21), mean (s.d.) | | | | |
| B12 (pg ml⁻¹) | 859.79 | (447.54) | 725.39 | (368.06) |
| (normal 200-900), mean (s.d.) | | | | |
| Zinc (mg dl⁻¹) | 109.64 | (31.64) | 99.00 | (19.29) |
| (normal 65-120), mean (s.d.) | | | | |
| Copper (μg dl⁻¹) | 103.10 | (12.56) | 109.19 | (17.07) |
| (normal 70-128), mean (s.d.) | | | | |
| Magnesium (μg g⁻¹) | 42.15 | (10.60) | 47.77 | (11.50) |
| (normal 39-59 I), mean (s.d.) | | | | |
| Language testing, N (%) | | | | |
| Preverbal at start of study | 8 | (35) | 11 | (44) |
| Preschool Language Scales | 3 | (14) | 5 | (20) |
| Clinical Evaluation of Language | 14 | (62) | 12 | (48) |
| Fundamentals 2 | | | | |
| Clinical Evaluation of Language | 6 | (26) | 8 | (32) |
| Fundamentals 4 | | | | |
| Diagnostic Documentation, N | | | | |
| (%) | | | | |
| Autism Diagnostic Observation | 12 | (52) | 16 | (64) |
| Schedule | | | | |
| 3 Practitioner Agreement | 18 | (78) | 21 | (84) |
| Single practitioner with | 2 | (9) | 1 | (4) |
| standardized questionnaires^{b} | | | | |
| Autism Diagnostic Interview- | | | | |
| Revised | | | | |
| Participated in confirmation | 19 | (83) | 21 | (84) |
| testing, N (%) | | | | |
| Social Interaction Score, mean | 20.50 | (5.50) [10-27] | 23.80 | (4.80) [11-30] |
| (s.d.) [range] | | | | |
| Communication Score: Verbal, | 17.00 | (3.92) [10-22] | 19.00 | (5.16) [7-25] |
| mean (s.d.) [range] | | | | |
| Communication Score: Non- | 12.80 | (2.68) [8-14] | 13.40 | (0.52) [13-14] |
| Verbal, mean (s.d.) [range] | | | | |
| Restricted & Repetitive Play | 4.78 | (1.86) [2-9] | 6.40 | (2.08) [2-12] |
| Score, mean (s.d.) [range] | | | | |
| Summary Score, mean (s.d.) | 4.22 | (1.06) [2-5] | 4.25 | (0.91) [3-5] |
| [range] | | | | |
| Medications (concurrent | | | | |
| treatments), N (%) | | | | |
| Stimulant | 6 | (26) | 6 | (24) |
| Melatonin | 6 | (26) | 5 | (20) |
| Allergy/asthma medications | 4 | (17) | 7 | (28) |
| Gastrointestinal medications | 4 | (17) | 5 | (20) |
| Alpha-adrenergic agonists | 5 | (22) | 3 | (12) |
| Selective serotonin reuptake | 3 | (13) | 1 | (4) |
| inhibitors | | | | |
| Antiepileptic medication | 2 | (9) | 2 | (8) |
| Antimicrobial medications | 3 | (13) | 0 | (0) |
| Immunomodulatory | 1 | (4) | 0 | (0) |
| medications | | | | |
| Supplements (concurrent | | | | |
| treatments), N (%) | | | | |
| Multivitamin | 3 | (13) | 11 | (44) |
| Minerals | 4 | (17) | 3 | (12) |
| Vitamin B12 | 2 | (9) | 4 | (16) |
| Other B vitamins | 2 | (9) | 2 | (8) |
| Fatty acids | 1 | (4) | 3 | (12) |
| Other antioxidants | 1 | (4) | 2 | (8) |
| Folate | 0 | (0) | 3 | (12) |
| Carnitine | 1 | (4) | 1 | (4) |
| Coenzyme Q10 | 1 | (4) | 0 | (0) |
| Amino acids | 1 | (4) | 0 | (0) |
| Other vitamins | 0 | (0) | 1 | (4) |
| Comorbid medical | | | | |
| conditions, N (%) | | | | |
| Allergic disorders | 9 | (39) | 12 | (48) |
| Gastrointestinal disorders | 9 | (39) | 10 | (40) |
| Neurological disorders | 6 | (26) | 10 | (40) |
| Sleep disorders | 4 | (17) | 8 | (32) |
| Other psychiatric disorders | 6 | (26) | 4 | (16) |
| Immune abnormality^{c} | 8 | (35) | 5 | (20) |
| Mild congenital malformations | 1 | (4) | 1 | (4) |
| Genetic disorders^{d} | 1 | (4) | 0 | (0) |

Mean values with standard deviation in parenthesis and range in brackets.
^{a}Baseline outcome measures outlined in Table 1 were not significantly different across treatment groups.
^{b}Standardized questionnaires included Social Responsiveness Scale, Social Communication Questionnaire and/or Autism Symptoms Questionnaire.
^{c}Immune abnormalities include chronic ear infection in 9 (19%) of participants (6 (26%) placebo, 3 (12%) folinic acid), urinary tract infections in 2 (4%) of participants (1 (4%) placebo, 1 (4%) folinic acid), chronic infections in 2 (4%) of participants (0 (0%) placebo, 2 (8%) folinic acid), immunological disorder in 1 (2%) of the participants (1 (4%) placebo, 0 (0%) folinic acid), adenotonsillar hypertrophy in 1 (2%) of the participants (1 (4%) placebo, 0 (0%) folinic acid) and PANDAS/PANS in in 1 (2%) of the participants (1 (4%) placebo, 0 (0%) folinic acid).
^{d}During the study a child was found to have a phosphatase and tensin homolog gene mutation.

### Primary Outcome

Improvement in verbal communication was significantly greater for the participants on folinic acid as compared with participants on placebo with a medium-to-large effect size (Cohen's d=0.70) (Table 2A).

Separate analyses were conducted for each biomarker of folate metabolism (Table 2A). In general, improvement in verbal communication was significantly greater in participants on folinic acid as compared with those on placebo for participants with abnormal folate metabolism (i.e., FRAA positive, low glutathione redox ratio). For participants with biomarkers indicating more normal folate metabolism (i.e., FRAA negative, high glutathione redox ratio) improvement in verbal communication was not significantly different between groups.

TABLE 2A
Statistical analysis of primary outcome measure of verbal communication mixed model analysis
(standardized score, 95% confidence interval shown)

| | | Folinic acid | | Placebo | | | | |
|---|---|---|---|---|---|---|---|---|
| | | | 12 | | 12 | Estimated | Effect | |
| | N | Baseline | weeks | Baseline | weeks | Effect^{a} | size^{b} | P-value |
| Overall | 48 | 58.1 (53.9, | 65.4 | 56.8 | 58.5 | 5.7 (1.0, | 0.70 | 0.02 |
| | | 62.1) | (60.6, | (51.5, | (51.9, | 10.4) | | |
| | | | 70.2) | 62.2) | 65.1) | | | |
| Antibody | | | | | | | | |
| status | | | | | | | | |
| Negative | 17 | 57.9 (50.6, | 64.5 | 48.0 | 52.1 | 2.5 (−5.9, | 0.30 | 0.58 |
| | | 65.2) | (57.3, | (45.5, | (45.5, | 10.9) | | |
| | | | 71.7) | 50.5) | 58.8) | | | |
| Positive | 31 | 58.1 (52.9, | 66.1 | 60.3 | 60.9 | 7.3 (1.4, | 0.91 | 0.02 |
| | | 63.3) | (59.0, | (53.5, | (52.2, | 13.2) | | |
| | | | 73.1) | 67.0) | 69.6) | | | |
| Glutathione | | | | | | | | |
| ratio | | | | | | | | |
| High | 24 | 59.2 (53.6, | 65.0 | 55.1 | 58.1 | 3.0 (−2.5, | 0.46 | 0.30 |
| | | 64.8) | (58.4, | (47.1, | (50.0, | 8.5) | | |
| | | | 71.6) | 63.1) | 66.2) | | | |
| Low | 24 | 56.1 (49.6, | 66.0 | 58.0 | 58.7 | 9.1 (0.9, | 0.95 | 0.04 |
| | | 62.7) | (58.2, | (50.5, | (28.7, | 17.3) | | |
| | | | 73.8) | 65.5) | 68.7) | | | |

^{a}Estimated effect of folinic acid treatment with 95% confidence interval. Estimated effect is the difference in the outcome measures between the folinic acid and placebo group as estimated by the mixed-model regression.
^{b}Cohen's d effect size is a measure of the strength of the effect of the folinic acid intervention. Higher values represent stronger effects. For the Cohen's d, 0.25 is a small effect, 0.5 is a medium effect and 0.8 is a large effect.

A responder analysis was also performed. Overall, there were significantly more responders in the folinic acid group as compared with those on placebo (X²(1)=8.92, P=0.003; Table 2B). FRAAs predicted response to folinic acid (X²(1)=4.92, P=0.03). For both analyses, greater baseline Adaptive Behavior Composite Score increased the likelihood of response (X²(1)=6.92, P=0.009 and X²(1)=7.74, P=0.005, respectively) but all other potential covariates were removed by backward elimination. Glutathione redox status was not significantly associated with treatment response.

TABLE 2B
Statistical analysis of primary outcome measure of verbal communication responder analysis^{a}

| | | | | | | Adjusted | | |
|---|---|---|---|---|---|---|---|---|
| | | | % | Unadjusted | | odds | | Number |
| | Folinic acid | Placebo | Difference | odds | P- | ratio | P- | needed |
| | treatment^{b,c} | treatment^{b,d} | responders | ratio and | value | and | value | to treat |
| Overall | 15 65% | 6 24% (7%, | 41% (13%, | 5.9 (1.7, | 0.01 | 14.9 | 0.003 | 2.4 (1.6, |
| | (46%, 84%) | 41%) | 63%) | 20.9) | | (2.1, | | 7.7) |
| | | | | | | 116.9) | | |
| Antibody | | | | | | | | |
| status | | | | | | | | |
| Negative | 5 50% (19%, | 2 29% (−4%, | 21% | 2.5 (0.3, | 0.32 | 3.6 (0.1, | 0.45 | 4.7 (1.5, |
| | 81%) | 62%) | (−27%, | 19.5) | | 95.6) | | −4.1) |
| | | | 60%) | | | | | |
| Positive | 10 77% | 4 22% (3%, | 55% (19%, | 11.7 (2.1, | 0.005 | 67.4 | 0.01 | 1.8 (1.3, |
| | (54%, 99%) | 41%) | 78%) | 64,0) | | (5.6, | | 5.2) |
| | | | | | | 999.9) | | |
| Glutathione | | | | | | | | |
| ratio | | | | | | | | |
| High | 9 64% (39%, | 3 30% (2%, | 34% (−4%, | 4.2 (0.73, | 0.11 | 21.9 | 0.04 | 2.9 (1.4, |
| | 89%) | 58%) | 72%) | 23.9) | | (1.9, | | −27.6) |
| | | | | | | 956.9) | | |
| Low | 6 67% (36%, | 3 20% (0%, | 47% (10%, | 8.0 (1.2, | 0.03 | 10.2 | 0.04 | 2.1 (1.2, |
| | 98%) | 40%) | 84%) | 52.2) | | (1.4, | | 10.2) |
| | | | | | | 140.6) | | |

^{a}Response is defined as an increase in five standardized points on the primary outcome, which was measured using the Preschool Language Scales-5, the Clinical Evaluation of Language Fundamentalspreschool-2 or Clinical Evaluation of Language Fundamentals 4.
^{b}Number of responders % responders (95% confidence interval)).
^{c}Overall N = 23; antibody negative = 10; antibody positive = 13; glutathione high = 14; glutathione low = 9.
^{d}Overall N = 25; antibody negative = 7; antibody positive = 18; glutathione high = 10; glutathione low = 15.

### Secondary Outcomes

Table 3 outlines secondary outcomes, including the minimal clinically important difference. The Daily Living Skills on the VABS significantly improved in the folinic acid group as compared with the placebo group.

TABLE 3
Secondary outcome measures

| | | | 12-week assessment | | | | |
|---|---|---|---|---|---|---|---|
| | | | Folinic | | | | |
| | Baseline | | acid, | Placebo, | | | |
| | Folinic acid, | Placebo, | Mean | Mean | Estimated | | P- |
| Measure^{a} | Mean (CI^{b}) | Mean (CI^{b}) | (CI^{b}) | (CI^{b}) | effect^{c} | MCID^{d} | value^{e} |

Vineland Adaptive Behavior Scale (Standard Score): Higher Scores = Better Performance

| | | | | | | | |
|---|---|---|---|---|---|---|---|
| Communication | 66.2 (62.0, | 65.9 (60.8, | 68.3 | 66.0 (59.8, | 0.2 (0.4, | 3.8 | 0.87 |
| | 70.4) | 71.1) | (63.5, | 72.2) | −0.2) | | |
| | | | 73.2) | | | | |
| Daily living | 64.6 (61.2, | 68.1 (62.5, | 69.2 | 66.3 (60.3, | 0.5 (0.0, | 2.8 | 0.05 |
| | 68.0) | 73.7) | (64.4, | 72.3) | 1.0) | | |
| | | | 74.0) | | | | |
| Social skills | 64.4 (60.8, | 66.1 (61.9, | 68.3 | 67.4 (61.7, | 0.2 (−0.2, | 3.0 | 0.29 |
| | 68.0) | 70.4) | (64.2, | 73.2) | 0.6) | | |
| | | | 72.5) | | | | |
| Motor skills | 78.9 (72.5, | 78.5 (73.0, | 81.7 | 80.6 (74.5, | 0.1 (−0.1, | 4.4 | 0.69 |
| | 85.3) | 84.0) | (75.3, | 86.7) | 0.3) | | |
| | | | 88.2) | | | | |
| Adaptive | 64.8 (61.6, | 65.8 (62.0, | 67.7 | 65.8(60.8, | 0.3 (0.7, | 2.4 | 0.16 |
| behavior | 68.0) | 69.7) | (63.7, | 70.8) | −0.1) | | |
| | | | 71.7) | | | | |

Aberrant Behavior Checklist (Raw Score): Lower Scores = Less Behavioral Problems

| | | | | | | | |
|---|---|---|---|---|---|---|---|
| Irritability | 13.4 | 10.2 (7.5, | 9.1 (7.1, | 8.5 (5.7, | −1.2 | 1.8 | 0.04 |
| | (10.3, 16.5) | 13.0) | 11.0) | 11.4) | (−0.2, | | |
| | | | | | −2.2) | | |
| Lethargy | 13.1 | 13.1 (9.9, | 9.7 (7.2, | 11.1 (7.7, | −1.4 | 1.0 | 0.02 |
| | (10.8, 15.4) | 16.2) | 12.2) | 14.6) | (−1.0, | | |
| | | | | | −1.9) | | |
| Stereotyped | 6.1 (4.5, 7.7) | 7.3 (5.2, 9.5) | 3.7 (2.5, | 7.1 (5.1, | −0.9 | 0.7 | 0.007 |
| behavior | | | 5.0) | 9.1) | (−0.4, | | |
| | | | | | −1.4) | | |
| Hyperactivity | 25.0 (21.8, | 18.5 (13.5, | 22.1 | 16.0 (11.6, | −1.8 | 2.6 | 0.02 |
| | 28.1) | 23.5) | (17.5, | 20.4) | (−0.6, | | |
| | | | 26.4) | | −3.0) | | |
| Inappropriate | 5.2 (3.8, 6.6) | 4.1 (2.6, 5.6) | 4.0 (3.0, | 3.8 (2.6, | −1.7 | 0.7 | 0.004 |
| speech | | | 5.0) | 5.1) | (−0.9, | | |
| | | | | | −2.5) | | |
| Total Score | 62.7 | 53.2 (43.2, | 48.5 | 46.6 (35.9, | −4.7 | | 0.02 |
| | (54.9, 70.6) | 63.2) | (40.9, | 57.2) | (−3.0, | | |
| | | | 56.1) | | −6.4) | | |

The Ohio Autism Clinical Impression Scale (Severity): Lower Scores = Less Severity and Greater
Improvement

| | | | | | | | |
|---|---|---|---|---|---|---|---|
| Social | 4.3 (4.0, | 4.4 (4.0, 4.8) | 3.8 (3.3, | 4.0 (3.4, | −0.1 | 0.4 | 0.52 |
| | 4.6) | | 4.4) | 4.5) | (−0.3, | | |
| | | | | | 0.1) | | |
| Aberrant | 3.8 (3.3, | 3.6 (3.2, 4.0) | 3.4 (2.9, | 3.5 (3.0, | −0.3 | 0.4 | 0.32 |
| behavior | 4.2) | | 3.8) | 4.0) | (−0.6, | | |
| | | | | | 0.0) | | |
| Repetitive | 3.2 (2.8, | 2.8 (2.3, 3.3) | 2.5 (2.2, | 2.7 (2.2, | −0.4 | 0.4 | 0.13 |
| behavior | 3.5) | | 2.8) | 3.2) | (−0.8, | | |
| | | | | | 0.0) | | |
| Verbal | 4.4 (3.9, | 4.7 (4.1, 5.3) | 3.8 (3.2, | 4.3 (3.7, | −0.2 | 0.4 | 0.37 |
| communication | 4.8) | | 4.3) | 5.0) | (−0.6, | | |
| | | | | | 0.2) | | |
| Non-verbal | 3.6 (3.2, | 3.8 (3.3, 4.4) | 3.3 (2.8, | 3.5 (3.0, | −0.0 | 0.4 | 0.63 |
| communication | 4.0) | | 3.8) | 4.1) | (−0.4, | | |
| | | | | | 0.4) | | |
| Hyperactivity | 4.1 (3.8, | 3.7 (3.2, 4.1) | 3.8 (3.3, | 3.8 (3.3, | −0.3 | 0.4 | 0.29 |
| | 4.4) | | 4.3) | 4.3) | (−0.9, | | |
| | | | | | 0.3) | | |
| Anxiety | 2.5 (2.0, | 2.1 (1.5, 2.7) | 2.3 (1.7, | 2.0 (1.4, | −0.1 | 0.4 | 0.52 |
| | 2.9) | | 2.9) | 2.5) | (−0.5, | | |
| | | | | | 0.3) | | |
| Sensory | 1.6 (1.4, | 1.2 (1.0, 1.5) | 1.4 (1.2, | 1.4 (1.0, | −0.3 | 0.4 | 0.15 |
| sensitivity | 1.9) | | 1.7) | 1.8) | (−0.6, | | |
| | | | | | 0.0) | | |
| Restricted | 2.3 (1.9, | 2.6 (2.3, 3.0) | 2.0 (1.6, | 2.0 (1.7, | −0.2 | 0.4 | 0.87 |
| interest | 2.8) | | 2.3) | 2.4) | (−0.6, | | |
| | | | | | 0.2) | | |
| Autistic | 4.4 (4.0, | 4.6 (4.2, 5.0) | 4.0 (3.5, | 4.4 (2.9, | −0.2 | 0.4 | 0.37 |
| behavior | 4.8) | | 4.5) | 5.0) | (−0.6, | | |
| | | | | | 0.2) | | |

Autism Symptoms Questionnaires (Raw Score) Lower Scores = Less Autism Symptoms

| | | | | | | | |
|---|---|---|---|---|---|---|---|
| Social | 3.5 (3.1, | 3.6 (3.3, 3.9) | 3.3 (2.8, | 3.4 (3.0, | −0.1 | 0.11 | 0.10 |
| | 3.9) | | 3.8) | 3.8) | (0.0, | | |
| | | | | | −0.2) | | |
| Communication | 4.7 (4.4, | 4.6 (4.3, 4.8) | 4.7 (4.5, | 4.7 (4.5, | 0.0 | 0.09 | 0.51 |
| | 5.0) | | 4.9) | 4.9) | (−0.1, | | |
| | | | | | 0.1) | | |
| Stereotypic | 3.1 (2.8, | 3.4 (3.1, 3.8) | 3.2 (2.8, | 3.6 (3.3, | −0.2 | 0.12 | 0.02 |
| behavior | 3.4) | | 3.6) | 3.9) | (−0.1, | | |
| | | | | | −0.3) | | |
| Total score | 11.3 | 11.6 (11.1, | 11.2 (10.5, | 11.7 (11.1, | −0.3 | 0.04 | 0.02 |
| | (10.6, | 12.1) | 11.9) | 12.3) | (−0.1, | | |
| | 12.0) | | | | −0.5) | | |

Social Responsiveness Scale (T-Scores): Lower Scores = Less Social Symptoms

| | | | | | | | |
|---|---|---|---|---|---|---|---|
| Awareness | 79.2 | 76.9 (72.6, | 78.7 (74.9, | 75.0 (69.6, | 0.3 (1.9, | 7.1 | 0.52 |
| | (74.8, | 81.3) | 82.4) | 80.4) | −1.3) | | |
| | 83.7) | | | | | | |
| Cognition | 83.7 | 82.1 (78.7, | 79.3 (76.5, | 80.0 (76.3, | 0.8 | 5.8 | 0.37 |
| | (80.5, | 85.6) | 82.2) | 83.7) | (−0.6, | | |
| | 87.0) | | | | 2.2) | | |
| Communication | 82.5 | 84.3 (81.1, | 80.1 (75.9, | 77.7 (72.7, | 1.5 | 4.2 | 0.11 |
| | (78.2, | 87.6) | 84.4) | 82.7) | (−0.1, | | |
| | 86.8) | | | | 3.1) | | |
| Motivation | 75.5 | 78.9 (74.9, | 70.3 (65.8, | 73.0 (68.5, | 0.2 | 5.7 | 0.52 |
| | (70.4, | 82.6) | 74.9) | 77.6) | (−0.8, | | |
| | 80.6) | | | | 1.2) | | |
| Mannerisms | 83.4 | 84.2 (80.8, | 80.5 (76.1, | 79.8 (75.9, | 0.9 | 5.5 | 0.29 |
| | (79.6, | 87.7) | 85.0) | 83.8) | (−0.9, | | |
| | 87.2) | | | | 2.7) | | |
| Total | 84.9 | 85.8 (83.0, | 82.2 (79.0, | 80.7 (76.9, | 0.8 | | 0.29 |
| | (81.7, | 88.6) | 85.4) | 84.6) | (−0.8, | | |
| | 88.1) | | | | 2.4) | | |

Autism Impact Measure (Raw Scores): Lower Scores = Less Impact of Autism on the Family

| | | | | | | |
|---|---|---|---|---|---|---|
| Frequency | 135 (126, | 143 (137, 149) | 114 (105, | 125 (113, | −2.2 | 0.16 |
| | 144) | | 123) | 136) | (0.8, | |
| | | | | | −5.2) | |
| Impact | 105 (94, | 123 (111, 136) | 87 (76, | 103 (89, | 3.4 (7.0, | 0.11 |
| | 116) | | 99) | 118) | −0.2) | |

Behavioral Assessment System for Children (T-Scores): Lower problems scores and higher skills scores
are better

| | | | | | | | |
|---|---|---|---|---|---|---|---|
| Externalizing | 60.9 (57.6, | 53.4 (49.8, | 57.7 (54.8, | 51.6 (48.1, | −0.1 | 3.7 | 0.52 |
| problems | 64.2) | 56.9) | 60.6) | 55.1) | (−0.9, | | |
| | | | | | 0.7) | | |
| Internalizing | 49.3 (45.1, | 48.6 (43.4, | 43.1 (40.0, | 45.5 (41.5, | −1.1 | 3.8 | 0.05 |
| problems | 53.5) | 53.7) | 46.2) | 49.4) | (−0.2, | | |
| | | | | | −2.0) | | |
| Behavior | 71.1 (68.1, | 66.0 (62.5, | 65.3 (62.3, | 61.2 (57.3, | 0.1 | 3.7 | 0.63 |
| problems | 74.1) | 69.6) | 68.2) | 65.0) | (−0.9, | | |
| | | | | | 1.1) | | |
| Adaptive skills | 24.5 (22.0, | 25.0 (22.2, | 27.8 (25.3, | 26.1 (22.4, | 0.4 (0.0, | 3.8 | 0.87 |
| | 27.0) | 27.7) | 30.4) | 29.7) | 0.8) | | |

^{a}Adherence to completing questionnaires: folinic acid 50/60 (83%), placebo 64/73 (88%).
^{b}95% confidence interval.
^{c}Estimated effect of folinic acid treatment with 95% confidence interval. Statistically significant effects (P ≤ 0.05) are bold. Estimated effect is the difference in the outcome measures between the folinic acid and placebo group as estimated by the mixed-model regression.
^{d}The Minimal Clinically Important Difference (MCID) was calculated for each index using the standard error of measurement method. Reliability and standard deviations from autistic samples were used for the calculation. The MCID is defined as smallest change in the outcome that a patient would identify as important.
^{e}P-values adjusted using false discovery rate.

Adherence on the parental questionnaires was not significantly different across treatment groups. Irritability, lethargy, stereotyped behavior, hyperactivity, inappropriate speech and total score on the ABC significantly improved in the folinic acid group as compared with the placebo group. Stereotypic behavior and total score significantly improved for the folinic acid group as compared with the placebo group on the ASQ. Internalizing problems significantly improved for the folinic acid group as compared with the placebo group on the BASC.

Teacher questionnaires were not analyzed since adherence was below 35%.

### Adverse Events

There were no serious adverse events in the folinic acid group. One child on placebo was unblinded and removed from the study because of a potential serious adverse event. Three placebo participants underwent dose reduction. There were no significant group differences between adverse event frequencies (Table 4).

TABLE 4
Incidence of adverse events by treatment group

| Adverse event, | Folinic acid | | Placebo group | | Overall | | Fisher |
|---|---|---|---|---|---|---|---|
| N (%) | group (n = 21)^{a} | | (n = 25) | | (n = 46)^{a} | | P |
| Excitement or | 6 | (29%) | 10 | (40%) | 16 | (35%) | 0.53 |
| agitation | | | | | | | |
| Insomnia | 6 | (29%) | 10 | (40%) | 16 | (35%) | 0.53 |
| Increased | 6 | (29%) | 9 | (36%) | 15 | (33%) | 0.75 |
| motor | | | | | | | |
| activity | | | | | | | |
| Restlessness | 3 | (14%) | 7 | (28%) | 10 | (22%) | 0.31 |
| Aggression | 2 | (10%) | 6 | (24%) | 8 | (17%) | 0.26 |
| Increased | 1 | (5%) | 6 | (24%) | 7 | (15%) | 0.11 |
| tantrums | | | | | | | |
| Involuntary | 2 | (10%) | 4 | (16%) | 6 | (13%) | 0.67 |
| movements | | | | | | | |
| Dry mouth, | 3 | (14%) | 1 | (4%) | 4 | (9%) | 0.32 |
| excessive thirst | | | | | | | |
| Decreased | 2 | (10%) | 2 | (8%) | 4 | (9%) | 1.00 |
| appetite | | | | | | | |
| Depression | 1 | (5%) | 3 | (12%) | 4 | (9%) | 0.61 |
| Gastroesopha- | 1 | (5%) | 3 | (12%) | 4 | (9%) | 0.61 |
| geal reflux | | | | | | | |
| Emotional | 1 | (5%) | 3 | (12%) | 4 | (9%) | 0.61 |
| lability | | | | | | | |
| Constipation | 2 | (10%) | 1 | (4%) | 3 | (7%) | 0.59 |
| Nasal | 0 | (0%) | 3 | (12%) | 3 | (7%) | 0.24 |
| congestion | | | | | | | |
| Confusion | 1 | (5%) | 1 | (4%) | 2 | (4%) | 1.00 |
| Stiffness | 1 | (5%) | 1 | (4%) | 2 | (4%) | 1.00 |
| Diarrhea | 1 | (5%) | 1 | (4%) | 2 | (4%) | 1.00 |
| Weight gain | 2 | (10%) | 0 | (0%) | 2 | (4%) | 0.20 |
| Headache | 2 | (10%) | 0 | (0%) | 2 | (4%) | 0.20 |
| Weight loss | 1 | (5%) | 0 | (0%) | 1 | (2%) | 0.46 |
| Drowsiness | 0 | (0%) | 1 | (4%) | 1 | (2%) | 1.00 |
| Sweating | 0 | (0%) | 1 | (4%) | 1 | (2%) | 1.00 |
| Decreased | 0 | (0%) | 0 | (0%) | 0 | (0%) | 1.00 |
| motor | | | | | | | |
| activity | | | | | | | |
| Tremors | 0 | (0%) | 0 | (0%) | 0 | (0%) | 1.00 |
| Blurred vision | 0 | (0%) | 0 | (0%) | 0 | (0%) | 1.00 |
| Increased | 0 | (0%) | 0 | (0%) | 0 | (0%) | 1.00 |
| salivation | | | | | | | |
| Nausea/ | 0 | (0%) | 0 | (0%) | 0 | (0%) | 1.00 |
| vomiting | | | | | | | |
| Dizziness | 0 | (0%) | 0 | (0%) | 0 | (0%) | 1.00 |
| Rash | 0 | (0%) | 0 | (0%) | 0 | (0%) | 1.00 |
| Any adverse | 12 | (57%) | 17 | (68%) | 29 | (63%) | 0.55 |
| effect | | | | | | | |

^{a}Two participants in the folinic acid group did not pick up the intervention so they never had a chance to report any adverse effects and were not included in the adverse effect frequency calculations.

### Discussion

This study found an improvement in an important core ASD symptom, verbal communication, in non-syndromic ASD children receiving high-dose folinic acid vs placebo, particularly in those participants who were positive for FRAAs. Improvement in a number of secondary outcomes was observed as well, with no significant adverse events. The effect of folinic acid is consistent with the therapeutic effect of early behavioral interventions.

Folinic acid may have positive effects on metabolism through multiple pathways (FIG. 1). First, folinic acid can normalize folate-dependent one-carbon metabolism. Second, unlike folic acid, the common oxidized synthetic form of folate, folinic acid can readily enter the folate cycle without being reduced by dihydrofolate reductase. Third, folinic acid can cross the blood-brain barrier using the reduced folate carrier when the FRα is blocked by FRAAs or is non-functional due to mitochondrial dysfunction or genetic mutations.

This study suggests that FRAAs predict response to high-dose folinic acid treatment. This is consistent with the notion that children with ASD and FRAAs may represent a distinct subgroup. Other factors such as genetic polymorphisms in folate-related genes or mitochondrial dysfunction may be important in determining treatment response but were not examined in this study. When methylcobalamin was combined with folinic acid, improvement in communication as well as glutathione redox status was found. Indeed, future studies will be needed to define factors that predict response to treatment, investigate optimal dosing and help understand whether other compounds could work synergistically with folinic acid.

This study had limitations. First, the small sample size may have resulted in the imbalance in baseline language scores for one subgroup and limited the sensitivity of the analyses to detect some treatment effects. Second, the single-site design only provides limited generalization of these results. Third, although no adverse events were identified, safety of this treatment requires further study since many folate studies utilize lower doses and a healthy population. Fourth, further studies will be needed to determine the optimal folinic acid dose. The oral bioavailability of folate is strongly influenced by the enteric microbiome⁶² but there is strong evidence that the enteric microbiome is altered in children with ASD.⁶³

This study also supports the notion that measurement of FRAAs prior to a trial of folinic acid may be helpful for predicting response. In our previous study we offered folinic acid treatment to patients positive for FRAAs without obtaining CSF folate concentration measurement. We continue to believe this is a reasonable alternative to a diagnostic lumbar puncture but should be accompanied by close follow-up with an experienced physician. Since ASD is likely a lifelong disorder the long-term adverse effect of any treatment is of concern. As folinic acid may become increasingly used to treat ASD in the future, short-term and long-term adverse effects should be studied in more detail to ensure safety.

### CONCLUSIONS

In this small trial of children with non-syndromic ASD and language impairment, treatment with high-dose folinic acid for 12 weeks resulted in improvement in measures of verbal communication as compared with placebo. These findings should be considered preliminary until treatment is assessed in larger multicenter studies with longer duration.

This research was supported, in part, by Lee Silsby Compounding Pharmacy (to REF), Autism Speaks (#8202 to EVQ), Brenen Hornstein Autism Research and Education Foundation (to REF), Fraternal Order of Eagles (to REF), the Autism Research Institute (to REF) and the Jane Botsford Johnson Foundation (to SJJ). This trial is registered on clinicaltrials.gov as NCT01602016.

What claimed is:

1. A method of treating suffering from a CNS disorder, comprising administering a reduced folate, or a derivative, prodrug, active metabolite, stereoisomer, polymorph, analogue, or pharmaceutically acceptable salt thereof, to a human suffering from a neurobehavioral CNS disorder in which the patient exhibits a folate deficiency, wherein the amount of reduced folate is therapeutically effective to improve at least one core symptom of the CNS disorder.

2. The method of claim 1, wherein the reduced folate is selected from the group consisting of folinic acid, MTHF, or a derivative, active metabolite, prodrug, stereoisomer, polymorph, analogue, pharmaceutically acceptable salts of any of the foregoing, and mixtures of two or more of the foregoing.

3. The method of claim 1, wherein the reduced folate is the pharmacologically active levo-isomer of d,l-leucovorin or a pharmaceutically acceptable salt thereof.

4. The method of claim 3, wherein the reduced folate is levoleucovorin calcium.

5. The method of claim 1, wherein the human is a child from about 3 years to about 14 years old suffering from ASD.

6. The method of claim 5, wherein the symptom is verbal communication.

7. The method of claim 1, wherein the reduced folate is incorporated into an oral formulation.

8. The method of claim 7, wherein the oral formulation is selected from the group consisting of a capsule, a tablet, a powder, a film, an emulsion, an aqueous liquid, and a suspension.

9. The method of claim 8, further comprising administering the oral formulation twice daily in divided doses.

10. The method of claim 1, wherein the reduced folate is incorporated into a dosage form which delivers from about 0.1 mg/kg/day to about 50 mg/kg/day.

11. The method of claim 10, wherein the dosage form is a film.

12. The method of claim 1, wherein the human is suffering from a condition selected from depression, Alzheimer's disease (AD), schizophrenia, bipolar disorder, ADHD, CFD, epilepsy, and Autism Spectrum Disorder (ASD).

13. The method of claim 1, wherein the folinic acid is levoleucovorin calcium, the doses of levoleucovorin calcium are administered on a twice-a-day basis, and the amount of levoleucovorin calcium administered in each dose is from about 0.25 mg to about 250 mg.

14. A method of improving verbal communication in children suffering from Autism Spectrum Disorder (ASD), comprising
assessing whether a child suffering from ASD is positive for folate receptor-α antibodies (FRAAs),
assessing the verbal communication of the child who is positive for FRAAs via an ability-appropriate instrument selected from the CELF-preschool-2, CELF-4 and Preschool Language Scale-5, or a combination thereof,
administering on a chronic basis a therapeutically effective dose of folinic acid, MTHF, or a derivative, active metabolite, prodrug, stereoisomer, polymorph, analogue, a pharmaceutically acceptable salt of any of the foregoing, or a mixture of two or more of the foregoing, to the human child if the result of the verbal communication assessment indicates that the child is language impaired,
and re-assessing the verbal communication of the child via the ability-appropriate instrument in order to determine efficacy of the treatment.

15. The method of claim 14, further comprising administering a reduced amount of a target dose of the folinic acid to the child during the first two weeks of therapy.

16. The method of claim 14, further comprising administering about one-half of a target dose of the folinic acid to the child during the first two weeks of therapy.

17. The method of claim 14, further comprising adjusting the dose of the folinic acid in order to obtain further improved verbal communication of the child.

18. The method according to claim 1, wherein the method does not include the step of measuring folate concentration in the cerebrospinal fluid.

19. The method of claim 18, further comprising
assessing the verbal communication of the child who is positive for FRAAs via an ability-appropriate instrument selected from the CELF-preschool-2, CELF-4 and Preschool Language Scale-5, or a combination of two or more of these instruments,
administering on a chronic basis a therapeutically effective dose of folinic acid, MTHF, or a derivative, active metabolite, prodrug, stereoisomer, polymorph, analogue, a pharmaceutically acceptable salt of any of the foregoing, or a mixture of two or more of the foregoing, to the human child if the result of the verbal communication assessment indicates that the child is language impaired, and
re-assessing the verbal communication of the child via the ability-appropriate instrument in order to determine efficacy of the treatment.

20. A pharmaceutical composition for oral administration, comprising from about 0.25 to about 250 mg levoleuvorin calcium, and at least one pharmaceutically acceptable carrier for oral administration.
